(19) United States
(12) Patent Application Publication
(10) Pub. No.: US 2024/0052421 A1
(43) Pub. Date: Feb. 15, 2024

(54) METHOD OF IDENTIFYING AND TREATING MITOCHONDRIAL SUBTYPE TUMORS

(71) Applicant: The Trustees of Columbia University in the City of New York, New York, NY (US)

(72) Inventors: Antonio IAVARONE, Sunny Isles Beach, FL (US); Anna LASORELLA, New York, NY (US)

(21) Appl. No.: 18/259,067

(22) PCT Filed: Dec. 22, 2021
(86) PCT No.: PCT/US2021/064991

§ 371 Date: Jun. 22, 2023

Related U.S. Application Data
(60) Provisional application No. 63/130,199, filed on Dec. 23, 2020.

(51) Int. Cl. C12Q 1/6886

(52) U.S. Cl. CPC C12Q 1/6886; C12Q 2600/156; C12Q 2600/112; C12Q 2600/106

(57) ABSTRACT

The subject matter disclosed herein relates to a method of treating glioblastoma (GBM) in a subject in need thereof, the method comprising determining one or more GBM subtypes in a GBM sample via a pathway-based classifier approach and administering to the subject a pharmaceutically effective amount of an agent capable of modifying the activity of the biological pathways associated with the one or more GMB subtypes.

This application claims the benefit of and priority to U.S. Provisional Patent Application No. 63/130,199, filed on Dec. 23, 2020, the contents of which is hereby incorporated by reference in its entirety.

### GOVERNMENT SUPPORT

This invention was made with government support under grants CA101644, CA193313, CA131126, CA179044, CA190891, and CA239698 awarded by the National Institutes of Health. The government has certain rights in the invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosure of these publications in their entireties are hereby incorporated by reference into this application in order to more fully describe the state of the art as known to those skilled therein as of the date of the invention described herein.

### BACKGROUND

Glioblastoma, also known as glioblastoma multiforme (GBM), is the most aggressive type of cancer that begins within the brain. There is still no cure for GBM and the cause of most cases is unknows. Genetic and environmental factors are thought to play a role in disease pathogenesis.

### SUMMARY OF THE INVENTION

In certain aspects the subject matter described herein provides a method of treating glioblastoma (GBM) in a subject in need thereof, the method comprising: providing a GBM sample from the subject; determining a GBM subtype for the GBM sample; and administering to the subject a pharmaceutical composition, wherein the pharmaceutical composition modifies activity of one or more functional pathway associated with the GBM subtype.

In some embodiments, the GBM is IDH wild-type GBM. In some embodiments, the GBM subtype is a neurodevelopmental subtype. In some embodiments, the GBM subtype is neuronal (NEU). In some embodiments, the GBM subtype is proliferative/progenitor (PPR). In some embodiments, the GBM subtype is a metabolic subtype. In some embodiments, the GBM subtype is mitochondrial (MTC). In some embodiments, the MTC GBM subtype harbors deletions in at least a portion of chromosome 1p36.23. In some embodiments, the deletions in at least a portion of chromosome 1p36.23 comprise a deletion of gene SLC45A1. In some embodiments, the GBM subtype is glycolytic/plurimetabolic (GPM). In some embodiments, the GBM subtype comprises an FGFR3-TACC3 gene fusion.

In some embodiments, the pharmaceutical composition is an inhibitor of mitochondrial metabolism. In some embodiments, the pharmaceutical composition is an inhibitor of mitochondrial activity. In some embodiments, the pharmaceutical composition is an inhibitor of mitochondrial respiration. In some embodiments, the pharmaceutical composition is an OXPHOS inhibitor. In some embodiments, the pharmaceutical composition is IM-156. In some embodiments, the pharmaceutical composition is an inhibitor of mitochondrial complex I. In some embodiments, the pharmaceutical composition is metformin. In some embodiments, the pharmaceutical composition is IACS-010759. In some embodiments, the pharmaceutical composition is tigecycline. In some embodiments, the pharmaceutical composition is an inhibitor of mitochondrial protein translation. In some embodiments, the pharmaceutical composition is menadione. In some embodiments, the pharmaceutical composition is an inducer of mitochondrial ROS or apoptosis.

In some embodiments, the determining comprises a single cell RNA-seq analysis of the sample. In some embodiments, the determining comprises a scBiPaD analysis of the sample. In some embodiments, the determining comprises defining cluster-specific ranked-lists. In some embodiments, the determining comprises consensus clustering analysis of cell subpopulations in the sample.

In some embodiments, the determining comprises: generating a gene signature of the sample; comparing the gene signature to one or more gene signatures of GBM samples with known subtype; making a determination of the GBM subtype based on matching the gene signature to one or more known gene signature. In some embodiments, the determining further comprises a genomic analysis, a transcriptomic analysis, a DNA methylation analysis, a microRNA analysis, or a proteomics analysis of the sample.

In certain aspects the subject matter described herein provides a method of a determining clinical outcome in a subject having glioblastoma (GBM), the method comprising: providing a GBM sample from the subject; determining a GBM subtype for the GBM sample; and providing a clinical outcome based on the GBM subtype.

In some embodiments, the GBM is IDH wild-type GBM. In some embodiments, the GBM subtype is a neurodevelopmental subtype. In some embodiments, the GBM subtype is neuronal (NEU). In some embodiments, the GBM subtype is proliferative/progenitor (PPR). In some embodiments, the GBM subtype is a metabolic subtype. In some embodiments, the GBM subtype is mitochondrial (MTC). In some embodiments, the MTC GBM subtype harbors deletions in at least a portion of chromosome 1p36.23. In some embodiments, the deletions in at least a portion of chromosome 1p36.23 comprise a deletion of gene SLC45A1. In some embodiments, the GBM subtype is glycolytic/plurimetabolic (GPM).

In some embodiments, the determining comprises a single cell RNA-seq analysis of the sample. In some embodiments, the determining comprises a scBiPaD analysis of the sample. In some embodiments, the determining comprises defining cluster-specific ranked-lists. In some embodiments, the determining comprises consensus clustering analysis of cell subpopulations in the sample.

In some embodiments, the determining comprises: generating a gene signature of the sample; comparing the gene signature to one or more gene signatures of GBM samples with known subtype; making a determination of the GBM subtype based on matching the gene signature to one or more known gene signature. In some embodiments, the determining further comprises a genomic analysis, a transcriptomic analysis, a DNA methylation analysis, a microRNA analysis, or a proteomics analysis of the sample.

### BRIEF DESCRIPTION OF FIGURES

The patent or application file contains at least one drawing originally in color. To conform to the requirements for PCT patent applications, many of the figures presented herein are black and white representations of images originally created in color.

FIGS. 1A-B show combinatorial effects and prevention of drug resistance. FIG. 1A shows treatment with TAS120 or Metmorfin-TAS120 from day 0 until day 50. FIG. 1B shows re-start of treatment with TAS120 or Metmorfin-TAS120 between day 50 and day 100.

FIGS. 2A-F show identification of four core functional states in single glioma cells. FIG. 2A shows consensus clustering generated from clusters of 94 single-cell subpopulations from 17,367 cells (36 GBM tumors). Columns and rows represent cell subpopulations. Color bar on the left defines four cell clusters. Yellow-to-blue scale indicates low to high similarity. FIG. 2B shows a heatmap of biological activities of 94 single-cell subpopulations grouped by common activated pathways (2,533 out of 5,032 pathways; effect size >0.3, FDR<0.0001, two-sided MWW test). Columns represent cell subpopulations, rows are biological activities. Pathway activity levels are color coded. Representative pathways specifically activated in each subtype are indicated. Left and top color bars: red, GPM; green, MTC; blue, NEU; cyan, PPR. FIGS. 2C-F show an enrichment map network of statistically significant, nonredundant GO categories (log it(NES)>0.58, FDR<0.05, two-sided MWW-GST) in GPM (c), MTC (d), NEU (e) and PPR (f) medoids of each GBM state. FIG. 2C shows the right-hand side of the network was magnified 1.5-fold for better visualization of significant activities. Nodes represent gene ontology (GO) terms and lines their connectivity. Node size is proportional to the number of genes in the GO category, with range indicated by keys and line thickness indicating similarity coefficient. EMT, epithelial-mesenchymal transition; FA, fatty acids; CNS, central nervous system; ER, endoplasmic reticulum.

FIGS. 3A-E show that glioma cell states converge on metabolic and neurodevelopmental axes. FIG. 3A shows a spearman's correlation of GBM cell states within individual tumors. Rows and columns represent GBM cell states. The green-to-red scale indicates negative to positive correlation. Left and top color bars: red, GPM; green, MTC; blue, NEU; cyan, PPR. FIG. 3B shows a multidimensional scaling of cell state frequency in 36 tumors discriminating two clusters according to similarity: GPM-MTC (orange) and NEU-PPR (blue). Bar plots: frequency distribution of cell states in each cluster. FIG. 3C shows the percentage of cells in each subclass at the tumor core and periphery is indicated, and the variation between core and rim is represented by arrows. NEU cells are enriched at the rim (n=2,799 cells; P=2.2×10⁻¹⁶, χ2 test). FIG. 3D shows a stream plot showing cell density at tumor core and periphery from samples in C. The thickness of each branch is proportional to the number of cells in the branch. Bar plot, significant enrichment of cells from tumor periphery in branch S1-S2 (n=2,799 cells; P=2.2×10⁻¹⁶, χ2 test). FIG. 3E shows a stream plot showing subclasses of cells at tumor core and rim. Bar plot, significant enrichment of NEU cells at the tumor periphery (n=2,799 cells; P=2.2×10⁻¹⁶, χ2 test). Arrows depict two largely independent branches of cell states, NEU—PPR (neurodevelopment) and GPM-MTC (metabolic).

FIGS. 4A-F show classification of primary human GBM and clinical validation. FIG. 4A shows a heatmap of pathway activity in 304 GBM tumors using 2,792 of 5,032 pathways, showing differential activity in the four GBM subtypes (effect size >0.3 and FDR <0.01, two-sided MWW test). Columns represent tumors, rows are pathway activities. Representative pathways specifically activated in each GBM subtype are indicated. Left and top color bars: red, GPM; green, MTC; blue, NEU; cyan, PPR. FIG. 4B shows a two-dimensional plot of GBM subtype enrichment scores (n=304 tumors). Each quadrant corresponds to one GBM subtype, and the position of dots (tumors) reflects the relative subtype-specific NES of each tumor as indicated on the x and y axes; color intensity reflects NES value. Tumors that do not fall within the corresponding subtype quadrant are colored gray. FIG. 4C shows that Kaplan-Meier curves of 302 patients with GBM stratified according to the four biological classes. Patients in the MTC subgroup exhibit significantly longer survival (log-rank test). FIG. 4D shows a relative HR of 302 patients with GBM estimated by Cox's proportional hazards model, including the activity of MTC, GPM, NEU and PPR as the covariate (shaded areas represent 95% CI). FIG. 4E shows a sankey diagram of GBM subtype assignment (n=304 tumors) according to either pathway-based or previously published classification: left, Phillips et al.11; right, Wang et al.3. FIG. 4F shows functional subtyping of primary and recurrent GBM (n=61 tumor pairs). The transition plot of primary and recurrent GBM subtypes shows an increased frequency of the NEU subtype at recurrence (P=0.05, χ2 test). The number of GBM in each class at diagnosis and recurrence is indicated, and variations between primary and recurrent samples are represented by arrows. Mes, mesenchymal; prolif, proliferative; pron, proneural.

FIGS. 5A-F show that reciprocal MTC and GPM activities are associated with coherent gain- and loss-of-function genetic alterations and predict risk of failure. FIG. 5A shows that mutations and/or CNVs significantly associated with GBM subclasses (n=496 tumors); P<0.05, two-sided Fisher's exact and χ2 test). Columns represent tumors and rows are genes. Horizontal top and vertical color bars: GBM subtypes; horizontal middle and bottom bars: white and gray, samples with or without mutation (middle) or CNV (bottom) data, respectively. Representative gene alterations specific to each GBM subtype are indicated by color: green, mutation; red, amplification; blue, deletion; orange, mutation/amplification; cyan, mutation/deletion. FIG. 5B shows a metabolic pathway enrichment analysis of amplifications (left) and deletions (right) in GBM subtypes. Red-to-blue scale, positive to negative enrichment (P value) of gene alterations in the pathway; *P<0.10, **P<0.05, ***P<0.01, two-sided Fisher's exact test. FIG. 5C shows Top: HR for patients with GBM according to Cox's proportional hazards model, testing the difference between GPM and MTC activities as the covariate (n=273 tumors, P=0.05; shaded area represents 95% CI). Middle: correlation analysis of MTC (blue) and GPM (red) activities in individual GBM (n=273 tumors, Spearman's correlation, p=−0.6, P=2.2×10⁻¹⁶). Bottom: fCNV gain and loss of mitochondrial- and glycolytic-related genes in MTC GBM and GPM GBM. The number of genes amplified/deleted in each tumor is color coded (amplifications, red to white; deletions, blue to white). In all panels, n=153 MTC and n=120 GPM tumors. FIG. 5D shows starburst plots comparing DNA methylation and gene expression for 10,337 unique genes. Dashed lines indicate P=0.01 (n=59 tumors, two-sided MWW test). The bottom right and top left areas of each plot include genes significantly hypermethylated and downregulated (purple) or hypomethylated and upregulated (orange), respectively, in the specific subtype. FIGS. 5E-F show that micro RNA gene target networks were significantly changed in subtypes MTC (E, green nodes) and GPM (F, red nodes) (n=294 tumors; log 2(fold change (FC))>0, P<0.0005, two-sided MWW test). For each miRNA, we report targets whose expression was anticorrelated with miRNA expression (n=294 tumors; Spearman's correlation, p<0 and P <0.05). Highlighted are miRNA targets of interest regarding the biology of subtypes MTC and GPM GBM. NFKB, nuclear factor kappa B; Wnt, wingless-related integration site.

FIGS. 6A-I show that divergent metabolic activities support MTC and GPM PDC subtypes. FIG. 6A shows Basal, ATP-linked and maximal OCR in MTC and GPM PDCs. Data are mean±s.d. of one representative experiment, including n=6 MTC PDCs each derived from an independent patient and n=6 GPM PDCs each derived from an independent patient; *P=0.0165 for ATP-linked OCR, **P=0.0063 for basal OCR and ***P=0.0024 for maximal OCR. FIG. 6B shows basal glycolysis in MTC and GPM PDCs. Data are mean±s.d. of one representative experiment, including n=6 MTC PDCs each derived from an independent patient and n=6 GPM PDCs each derived from an independent patient; *P=0.0129. FIG. 6C shows OCR/ECAR ratio of MTC and GPM PDCs. Data are mean±s.d. of one representative experiment, including n=6 MTC PDCs each derived from an independent patient and n=6 GPM PDCs each derived from an independent patient; ***P=0.0002. FIG. 6D rate of glucose uptake in MTC and GPM PDCs. Data are mean±s.d. of n=3 independent experiments for each PDC, each performed in triplicate. Bars on the right-hand side of the graph indicate mean±s.e.m. of values observed in the two sets of PDCs; n=7 MTC PDCs and n=7 GPM PDCs each derived from an independent patient; ***P=0.0028. FIG. 6E shows lactate secretion by MTC and GPM PDCs. Data are mean±s.d. of n=3 independent experiments for each PDC, each performed in triplicate. Bars on the right-hand side of the graph indicate mean±s.e.m. of values observed in the two sets of PDCs; n=7 MTC PDCs and n=7 GPM PDCs each derived from an independent patient; ***P=0.0020. FIG. 6F shows glutamine consumption by MTC and GPM PDCs. Data are mean±s.e.m. of n =3 independent experiments for each PDC, each performed in triplicate. Bars on the right-hand side of the graph indicate mean±s.e.m. of values observed in the two sets of PDCs; n=7 MTC PDCs and n=7 GPM PDCs each derived from an independent patient; ***P=0.000002. FIG. 6G shows an enrichment map network of statistically significant lipid metabolism-related GO categories (logit(NES)>0.58 and FDR<0.05, two-sided MWW-GST) in GPM GBM. Nodes represent GO terms and lines their connectivity. Node size is proportional to the number of genes in the GO category, while line thickness indicates similarity coefficient. FIG. 6H shows microphotographs of MTC (top) and GPM (bottom) PDCs stained by Bodipy 493/503 (green); nuclei were counterstained with DAPI (blue). Insets show higher-magnification images of the outlined areas. FIG. 61 shows concentration of triacylglycerol in MTC and GPM PDCs. Data are mean±s.d. of n=3 independent experiments for each PDC, each performed in triplicate. Bars on the right-hand side of the graph indicate mean±s.e.m. of values observed in the two sets of PDCs; n=7 MTC PDCs and n=7 GPM PDCs each derived from an independent patient; **P=0.0032. A-C, Experiments were assessed with a minimum of four technical replicates for each PDC. Each of these experiments was repeated independently two times with similar results. In all experiments, significance was established by two-tailed t-test, unequal variance.

FIGS. 7A-E show that the SLC45A1 glucose-proton symporter on chromosome 1p36.23 has tumor suppressor activity in mitochondrial GBM. FIG. 7A shows highest-scoring deletions of chromosomal regions significantly associated with GBM subclasses (n=487 tumors). Circles are color coded and size reflects −log 10(P value) of subtype enrichment; *P<0.10, **P<0.05, ***P<0.02, two-sided Fisher's exact test. Blue-to-red scale indicates positive to negative enrichment. FIG. 7B shows Top: raw copy number values of genes located on 1p36.23; bottom: homozygous, heterozygous and functional or nonfunctional events colored on a blue scale (lower right). Columns represent samples harboring at least one deleted gene, ordered by SLC45A1 deletion status. Deletion score of each gene by ComFocal (lower left). FIG. 7C shows genomic DNA PCR for ENO1 and SLC45A1 in U87, H423 and H502 cells. GAPDH is shown as control. FIG. 7D shows three-dimensional bubble plot showing the frequency of driver genetic alterations in primary and recurrent GBM harboring homozygous deletions of SLC45A1 (n=8 matched primary and recurrent GBM tumor pairs); left and right axes represent alterations occurring in primary and recurrent tumors, respectively; top axis, alterations shared by both tumors. The size of each bubble is proportional to the number of alterations. FIG. 7E shows a rank order plot of genes expressed in MTC versus GPM groups. Genes are ranked from left to right in increasing expression order. Blue dots indicate acid-base transporter differentially downregulated in TCGA GBM and single-cell datasets (n=175 tumors; n=1,338 cells from dataset 1; n=5,604 cells from dataset 2; n=2,429 cells from dataset 3; log 2(FC)<−0.3 and FDR<0.05, two-sided MWW test). bp, base pairs.

FIGS. 8A-I show analysis of SLC45A1 function in GBM cells. FIG. 8A shows quantification of pHi in MTC and GPM PDCs. Data are mean±s.d. of n≥3 independent experiments performed in seven MTC and five GPM PDCs, each derived from an independent patient and each assessed by four technical replicates . Bars on the right-hand side of the graph indicate mean±s.d. of the values observed in the two sets of PDCs; n=7 MTC and n=5 GPM PDCs each derived from an independent patient; ***P=0.000004. FIG. 8B immunoblot of FLAG-SLC45A1 in U87 (SLC45A1 wild type) and H502 cells (SLC45A1-deleted). FIG. 8C shows quantification of pHi in H502 and U87 cells expressing either SLC45A1 or the empty vector. Data are mean±s.d. from n=3 independent experiments for U87 and n=4 independent experiments for H502, each performed with three technical replicates (***P=0.0078 for vector versus SLC45A1 in H502 cells; NS, not significant). FIG. 8D shows representative images of colony formation of H502 and U87 cells treated as in C. FIG. 8E shows growth curves of independent cultures of cells expressing either SLC45A1 or the empty vector. Data are mean±s.d from one experiment (n=4 independent cultures; ***P=0.00001 for vector versus SLC45A1 in H502 cells. FIG. 8F shows representative microphotographs of PDC-002 and -064 (SLC45A1-deleted) and PDC-078 (SLC45A1 wild type) following ectopic expression of SLC45A1 or the empty vector. FIG. 8G shows quantification of sphere-forming assay for cells treated as described in F. Data are from two independent experiments, each performed with three independent infections (n=6 independent infections); ***P=0.0000005 for vector versus SLC45A1 in PDC-002 and ***P=0.0000001 in PDC-064). Box plots span the first to third quartiles and whiskers show 1.5× interquartile range. FIG. 8H shows immunoblot of FLAG-SLC45A1 and V5-SLC9A1 in PDC-002 (SLC45A1-deleted) expressing either SLC45A1, SLC9A1, SLC45A1 plus SLC9A1 or the empty vector. FIG. 8I shows quantification of sphere-forming assay for cells described in H. Data are mean±s.d. from one representative experiment; n=3 independent infections; ***P=0.0004, vector (vec) versus SLC45A1; ***P=0.0001, vector/SLC45A1 versus SLC45A1/SLC9A1. The experiment was repeated two times with similar results. In all experiments, significance was established by two-tailed t-test, unequal variance.

FIGS. 9A-K show MTC PDCs are distinctly sensitive to mitochondrial inhibition. FIGS. 9A-D shows viability curves of 13 MTC and ten GPM PDCs each derived from an independent patient treated with either IACS-010759 (A), metformin (B), tigecycline (C) or menadione (D). Data are mean±s.d. of n≥3 replicates for each PDC from one representative experiment. FIG. 9E shows Top: mitochondrial inhibitor sensitivity score for MTC (blue dots) and GPM (red dots) PDCs (n=13 MTC PDCs and n=10 GPM PDCs; Spearman's correlation, p=−0.74, P=7.563×10⁻⁵). Middle: correlation analysis of MTC (blue) and GPM (red) activity in PDCs (n=25 MTC PDCs and n=21 GPM PDCs; Spearman's correlation, p=−0.51, P=0.0003). Bottom: enrichment of fCNV gain and loss of mitochondrial and glycolytic-related genes in MTC PDCs (n=25) and GPM PDCs (n=21). The number of genes amplified/deleted in each tumor is color coded (amplifications, red to white; deletions, blue to white). FIGS. 9F and G show viability curves of nine MTC PDCs and nine GPM PDCs each derived from an independent patient treated with either FX-11 (F) or DEAB (G). Data are mean±s.d. from n≥4 replicates for each PDC from one representative experiment. FIG. 9H shows transcriptional regulatory network of GBM subtypes. Representative MRs with differential activity (n=304 tumors, n=2,799 cells from dataset 1, n=9,652 cells from dataset 2 and n=4,916 cells from dataset 3; two-sided MWW test, FDR<0.01) in TCGA and at least two out of three single-cell datasets are shown (two-sided MWW-GST test, log it(NES)>0.58, FDR<0.01). Nodes represent MRs and target genes, while lines represent interactions. FIG. 9I shows quantification of sphere-forming assay for two MTC and two GPM PDCs, each derived from an independent patient expressing two different short-hairpin RNAs for either PPARGC1A or the empty vector. Data are mean±s.d. from one representative experiment including n=3 independent infections for each PDC; **P=0.0010 and 0.0013 for PDC-002 shRNA1 and shRNA2 versus vector, respectively; **P=0.0030 and 0.0023 for PDC-026 shRNA1 and shRNA2 versus vector, respectively; *P=0.0285 for PDC-078 shRNA1 versus vector; *P=0.0142 for PDC-021 shRNA1 versus vector. FIG. 9J shows cell viability after irradiation of five MTC and five GPM PDCs each derived from an independent patient. Data are mean±s.d. of one representative experiment assessed by n=30 replicates for each PDC; **P=0.0022. FIG. 9K shows ROS quantification in MTC and GPM PDCs. Data are mean±s.d. of n=3 independent experiments for each PDC, each performed in triplicate. Bars on the right-hand side of the graph indicate mean±s.e.m. of values observed in the two sets of PDCs; n=7 MTC PDCs and n=7 GPM PDCs, each derived from an independent patient; ***P=0.00006. Experiments in A-D, F, G, I and J were repeated two times with similar results. In all experiments, significance was evaluated by two-tailed t-test, unequal variance.

FIG. 10 shows the computational framework of single cell biological pathway deconvolution (scBiPaD). Step 1: identification of cell sub-populations of cells in each individual tumor that share activation of similar biological functions; Step 2: determination of enriched biological pathways in each cell sub-population by defining cluster-specific ranked-lists; Step 3: identification of cell sub-populations that share coherent biological functions across multiple tumors. In Step 1-i, the ranked list for each cell in each tumor is obtained by standardizing and ranking genes. The activity matrix (NES) of all cells composing each tumor is obtained by calculating the single-sample activity of all the 5,032 biological pathways with ssMWW-GST (Step 1-ii) and used to generate the Euclidean distance between every pair of cells in each tumor (Step 1-iii). Finally, the cell sub-populations of each tumor are identified by applying the consensus clustering on the basis of the Euclidean distance of the NES (Step 1-iv). In the following step (Step 2-i), the MWW-score is used to generate a cluster-specific ranked-list of genes for each cell sub-population by comparing the expression profiles of the cells in the cluster with all other cells in the same tumor. The enriched biological pathways of each cell sub-populations are derived in Step 2-ii by using MWW-GST as in Step 1-ii. Each cell sub-population is then represented by a binary vector, with 1 indicating the enriched biological pathways (Step 3-i) and the binary matrix is used in Step 3-ii to derive the Jaccard distance. In the last step, 3-iii, cell sub-populations are clustered by Jaccard distance using consensus clustering.

FIGS. 11A-I show expression of subtype associated markers and mapping of marker genes on the population structure of neurodevelopmental subtype. FIG. 11A shows rank order plot of changes in genes expressed in MTC cells versus the other groups. Genes are ranked from left to right in increasing expression order. Red dots indicate mitochondrial respiratory complex I genes differentially expressed in each single cell dataset [n=2,799 cells for dataset 1, n=9,652 cells for dataset 2, n=4,916 cells for dataset 3; log 2(FC)>0.3 and FDR<0.05, two-sided MWW test]. Representative genes up-regulated in at least two out of three datasets are shown. Colors indicate complex I structural classes. FIG. 11B shows rank order plot of changes of genes expressed in NEU cells versus the other groups. Genes are ranked as in A. Red dots indicate neurotransmitter receptors differentially expressed in each single cell dataset [n=2,799 cells for dataset 1, n=9,652 cells for dataset 2, n=4,916 cells for dataset 3; log 2(FC)>0.3 and FDR<0.05, two-sided MWW test]. For each dataset, up-regulated genes representing distinct neurotransmitter receptor families are indicated by different colors. FIG. 11C shows rank order plot of changes of genes expressed in PPR cells versus the other groups. Genes are ranked as in A, B. Red dots indicate neural progenitor marker genes differentially expressed in each single cell dataset [n=2,799 cells for dataset 1, n=9,652 cells for dataset 2, n=4,916 cells for dataset 3; log 2(FC)>0.3 and FDR<0.05, two-sided MWW test]. Representative genes differentially expressed in at least two out of three single cell datasets are indicated. FIG. 11D shows sankey diagram showing subtype assignment of single glioma cells according to scBiPaD classification and the described cell states⁴. FIG. 11E shows a barplot of the number of tumors and states in each of the 36 samples of the single cell cohort. FIG. 11F shows a barplot showing functional cell state (at least 15% of cells in the sample) composition of 36 GBM samples. FIG. 11G shows stream plots of proliferation markers expressed by the PPR cells at the tumor core. FIG. 11H shows stream plots of neural progenitor markers. Expression overlaps with proliferation markers and is excluded from the more differentiated cells at the tumor periphery. The newly born neuron marker TBR1 is expressed in a subset of cells of the neurodevelopment branch. FIG. 11I shows stream plots of synaptic and neurotransmitter receptor genes in non-proliferative cells at the invasive rim. Color scale indicates the log 2 normalized expression of the indicated gene.

FIGS. 12A-F show analysis of survival-associated biological pathways in single glioma cells. FIG. 12A shows consensus clustering of 103 cell sub-populations from the three single cell datasets obtained using 192 biological pathways significantly associated with patient survival. Columns and rows are cell sub-populations. Left track: red, GPM; green, MTC; blue, NEU; cyan, PPR. FIG. 12B shows a heatmap of the biological activities of cell sub-populations in A. Each group was defined by shared activated pathways among the 5,032-pathway collection (n=103 cell sub-populations; effect size >0.3, FDR<0.0001, two-sided MWW test). Columns are cell sub-populations, rows are pathway activities. Pathway activity level is color-coded. Representative pathways specifically activated in each of the four functional subtypes are indicated. Left and upper tracks are as in A. FIG. 12C shows enrichment map network of statistically significant and not redundant GO categories [log it(NES)>0.58 and FDR<0.05, two-sided MWW-GST] in GPM; FIG. 12D shows MTC; FIG. 12E shows NEU; FIG. 12F shows PPR medoids. Nodes are GO terms and lines their connectivity. Node size is proportional to number of genes in the GO category; line thickness indicates similarity coefficient. The right-hand side of the network in c was magnified 1.5-fold for a better visualization of the significant activities.

FIGS. 13A-F show t-SNE plot visualization of tumors and functional cell states in single glioma cells. FIG. 13A shows t-SNE plot of malignant cells colored by tumor from dataset 1; FIG. 13B shows dataset 2; FIG. 13C shows dataset 3. FIG. 13D shows t-SNE plot of malignant cells from dataset 1 colored according to functional states; FIG. 13E shows t-SNE plot of malignant cells from dataset 2 colored according to functional states; FIG. 13F shows a t-SNE plot of malignant cells from dataset 3 colored according to functional states. Cells concordantly classified using 5,032 or 192 pathways are colored: red, GPM; green, MTC; blue, NEU; cyan, PPR; grey, cells not concordantly classified.

FIGS. 14A-G show characterization of biological subtypes of bulk primary GBM. FIG. 14A shows consensus clustering of 534 GBM on the activity of 192 survival-associated pathways (p<0.05, log-rank test). Columns and rows are individual tumors. Left track: red, GPM; green, MTC; blue, NEU; cyan, PPR; black, unclassified. FIG. 14B shows a heatmap of pathway activity in 304 classified GBM including 126 out of 192 survival-associated and differentially active pathways in the four GBM subtypes (effect size >0.3 and FDR<0.01, two-sided MWW test). Columns are individual tumors and rows are pathway activity. Pathways characteristically activated in each core subtype are indicated. Left and upper tracks: red, GPM; green, MTC; blue, NEU; cyan, PPR. FIG. 14C shows a heatmap of genes differentially expressed and up-regulated in GBM subtypes (n=304 tumors; Kruskal-Wallis analysis with post hoc correction by Nemenyi's test for multiple comparison; FDR<0.01 and log 2(FC)>0.5). Columns are individual tumors, rows are genes. Representative genes specifically up-regulated in each GBM subtype are indicated. Tracks are as in B. FIG. 14D shows rank order plot of changes of genes expressed in GBM NEU. Genes are ranked from left to right in increasing expression order. Red dots indicate neurotransmitter receptors differentially expressed in NEU tumors and cells (n=2,799 cells for dataset 1, n=9,652 cells for dataset 2, n=4,916 cells for dataset 3, n=304 tumors for TCGA dataset; log 2(FC)>0.3, FDR<0.05, two-sided MWW test). For each dataset, up-regulated genes in neurotransmitter receptor families are indicated by colors. FIG. 14E shows rank order plot of changes of genes expressed in GBM PPR. Genes are ranked as in D. Red dots indicate neural progenitor genes differentially expressed in each dataset (n=2,799 cells for dataset 1, n=9,652 cells for dataset 2, n=4,916 cells for dataset 3, n=304 tumors for TCGA dataset; log 2(FC)>0.3, FDR<0.05, two-sided MWW test). Representative genes differentially expressed in at least three datasets are indicated. FIG. 14F shows a heatmap showing the 50 highest scoring genes of the four GBM subtypes-specific signatures. Rows are genes and columns are tumors (n=304 tumors). Track are as in B, C. FIG. 14G shows a two-dimensional representation of GBM subtype enrichment scores (n=304 tumors). Quadrant are GBM subtypes, the position of dots (tumors) reflects the relative subtype-specific score of each tumor as indicated by x- and y-axes, and their color the subtype simplicity score. Gray, tumors that do not fall in the respective subtype quadrant.

FIGS. 15A-J show validation of the biological classification of GBM and comparison with established classifiers. Subtype-specific gene signatures were used to classify GBM from independent cohorts. FIG. 15A shows a heatmap of GBM from the TCGA cohort profiled by RNA-seq (n=129 tumors). FIG. 15B shows a heatmap of GBM from the CGGA cohort (n=94 tumors). FIG. 15C shows a heatmap of GBM69 (n=158 tumors). FIG. 15D shows Kaplan-Meier of patients in a (128 out of 129 patients with survival data available). FIG. 15E shows Kaplan-Meier of patients in B (90 out of 94 patients with survival data available). FIG. 15F shows Kaplan-Meier of patients in C (156 out of 158 patients with survival data available). Patients were stratified according to the four biological subtypes; survival differences were assessed using the log-rank test. FIG. 15G shows Kaplan-Meier of GBM patients from the TCGA cohort profiled by Agilent microarray (n=302 patients, log-rank test) and FIG. 15H GBM patients from the TCGA cohort profiled by RNA-seq (n=145 patients, log-rank test) classified according to mesenchymal, proneural and classical subtype. FIG. 15I Kaplan-Meier of GBM patients as in FIGS. 15G and J, patients as in H classified according to mesenchymal, proneural and proliferative subtype.

FIGS. 16A-D show analysis of the tumor microenvironment and GBM driver alterations in the biological GBM subtypes. FIG. 16A shows box plots of GBM subtypes tumor purity scores computed by ABSOLUTE; p-values: Kruskal-Wallis test with Nemenyi post hoc correction for multiple comparison (n=282 tumors). Box plots span the first to third quartiles and whiskers show the 1.5× interquartile range. FIG. 16B shows correlation analysis of non-tumor cell fraction in relationship with GBM cell state fraction (n=36 tumors; Spearman's correlation; p=0.089 GPM versus macrophages; p=0.067 GPM versus neutrophils; p=0.017 GPM versus oligodendrocytes; p=0.092 MTC versus macrophages; p=0.026 PPR versus oligodendrocytes; *p<0.10; **p<0.05). Rows are GBM cell states. Columns are non-tumor cell types. Blue to red scale indicates negative to positive correlation. FIG. 16C shows a heatmap of the expression of the top 25 microglia- and macrophage-specific genes in non-tumor cells from two GPM and two MTC GBM from single cell dataset 1. Cells are ordered by gene expression fold-change of macrophage—versus microglia-specific genes. The upper horizontal track shows in red and green non-tumor cells from GBM whose tumor cells have a dominant GPM (S4_D1, n=67 cells, and S12_D1, n=246 cells) or MTC state (S1_D1, n=65 cells, and S5_D1, n=29 cells), respectively. Representative microglia and macrophages marker genes are indicated. FIG. 16D shows bar plots showing the frequency distribution of GBM driver genes grouped by signaling pathways across GBM subtypes. Asterisks indicate the statistical significance (n=496 tumors; two-sided Fisher's exact test).

FIG. 17A-H show characterization of GBM biological states by multi-omics data analysis. FIG. 17A shows a heatmap of the M-values of the 100 probes most differentially methylated between GBM subtypes (n=59 tumors; two-sided MWW test, p<0.01 and absolute methylation log 2(fold-change) >0.58). FIG. 17B shows volcano plots of differentially expressed miRNA. Up-regulated miRNAs in each GBM subtype are indicated in red [n=294 tumors; log 2(FC)>0 and p-value <0.0005, two-sided MWW test]. Vertical and horizontal gray lines demarcate log 2(fold-change) and p-value cutoff, respectively. Representative miRNAs up-regulated in each functional subtype are indicated. FIG. 17C shows representative miRNA-gene targets networks significantly up-regulated in PPR and FIG. 17D shows NEU GBM subtypes [n=294 tumors; log 2(FC)>0 and p<0.0005, two-sided MWW test]. miRNA targets whose expression was anti-correlated with miRNA expression are listed (n=294 tumors; Spearman's correlation, p<0 and p<0.05) and biological pathways regulated by miRNA-target activity are indicated. Red nodes indicate miRNA targets of interest for the biology of the specific GBM subtype. Box plots showing the expression of selected proteins or phosphoproteins significantly up-regulated (n=103 tumors; two-sided MWW test) by RPPA in FIG. 17E GPM; FIG. 17F MTC; FIG. 17G NEU; FIG. 17H PPR GBM. Box plots span the first to third quartiles and whiskers show the 1.5× interquartile range.

FIGS. 18A-E show genomic and metabolic characterization of GBM PDCs. FIG. 18A shows classification of PDCs by random forest. Upper panel, bar plot showing mean±s.d. of NES of subtype-specific biological activity in each PDC subgroup. Middle panel, representative biological pathways exhibiting differential activity among subtypes [n=79 PDCs; log it(NES)>0.3 and FDR<0.05, two-sided MWW test]. Bottom panel, representative genes differentially expressed in PDC subtypes (n=79 PDCs; log 2(FC)>0.3 and FDR<0.05, two-sided MWW test). Red, green, blue, and cyan indicate significant pathway activation/gene up-regulation in PDCs classified as GPM, MTC, NEU or PPR, respectively; gray, pathway activation/gene up-regulation in any other subtype; white, lack of activation or up-regulation. FIG. 18B shows OCR kinetics in 2 MTC PDCs each derived from an independent patient and 2 GPM PDCs each derived from an independent patient shows elevated OCR in MTC PDCs. Data are mean±s.d. from one representative experiment for each PDC including n>9 replicates. FIG. 18C shows ECAR kinetics in 2 MTC PDCs each derived from an independent patient and 2 GPM PDCs each derived from an independent patient shows elevated glycolysis in GPM PDCs. Data are mean±s.d. from one representative experiment for each PDC including n>7 replicates. Experiments were repeated two times with similar results. FIG. 18D shows box plots showing the expression of SLC1A5 in GPM (n=7 tumors) and MTC (n=108 tumors) primary GBM. FIG. 18E shows box plots showing the expression of SLC1A5 in GPM and MTC PDCs (n=21 GPM PDCs each derived from an independent patient and n=25 MTC PDCs each derived from an independent patient); p-value: two-sided MWW test. Box plots span the first to third quartiles and whiskers show the 1.5 × interquartile range.

FIGS. 19A-H show that SLC45A1 is the target of chromosome 1p36.23 deletion in MTC GBM. FIG. 19A shows schematics of chromosome location peak deletions in MTC GBM (n=153 tumors) identified using GISTIC2 (Benjamini Hochberg FDR q-value <0.01). FIG. 19B shows the matrix of homozygous deleted genes identified by UNCOVER as associated with MTC NES in primary GBM (n=487 tumors; p=0.034, permutation test). Top row, blue to yellow: higher to lower NES values for samples (columns). Deletions in each sample are in dark blue; samples not deleted are in yellow. The last row shows the alteration profile from the entire analysis. The bar plot on the right side indicates the gene weight for each alteration. FIG. 19C shows association of homozygous deletions in each GBM subtype. Circles are color-coded and their dimension reflects the −log 10(p-value) of the enrichment (n=487 tumors; p-value, two-sided Fisher's exact test;). Blue to red scale indicates positive to negative relationship. FIG. 19D shows frequency of genetic alterations of GBM driver genes in SLC45A1-deleted (n=20 tumors) compared to SLC45A1 wild-type GBM (n=705 tumors). The bottom track indicates the dataset (green, TCGA; blue, GLASS). Asterisk, p=2.33e-03, two-sided Fisher's Exact test (n=725 tumors). FIG. 19E shows sample density plot depicting the relative frequency distribution of CCF estimated for the genetic alterations occurring in SLC45A1-deleted GBM (n=20 tumors). Blue dot, CCF of SLC45A1 deletion. FIG. 19F shows evolutionary trees of genetic alterations in primary and recurrent SLC45A1-deleted GBM (n=8 matched primary and recurrent tumor pairs); yellow, red and black branches are truncal, primary private and recurrent private alterations, respectively; the length of branches is proportional to the number of genetic alterations. GBM driver genes are indicated. FIG. 19G shows PCR amplification of genomic DNA shows deletion of SLC45A1 in PDC-002 and PDC-064. FIG. 19H shows immunoblot of FLAG- SLC45A1 in PDC-002, PDC-064 (harboring SLC45A1 deletion) and PDC-078 (SLC45A1 wild type). Experiments in G, H were repeated two times with similar results.

FIG. 20 shows distribution of glioblastoma patients-derived organoids (PDOs) by subtype for analysis of the efficacy of the OXPHOS inhibitor IM-156.

FIGS. 21A-F show activity of IM-156 in mitochondrial and glycolytic/plurimetabolic glioblastoma PDOs compared with other OXPHOS inhibitors. FIG. 21A shows viability ratios with IM-156 treatment. FIG. 21B shows viability ratios with IACS-010759 treatment. FIG. 21C shows viability ratios with menadione treatment. FIG. 21D shows viability ratios with metformin treatment. FIG. 21E shows viability ratios with tigecycline treatment. FIG. 21F shows IC₅₀ values for treatments of different GBM subtypes.

FIGS. 22A-C show IM-156 activity in glioblastoma including glycolytic/plurimetabolic PDOs. FIG. 22A shows viability rates with increasing IM-156 concentrations. FIG. 22B shows viability rates with increasing metformin concentrations. FIG. 22C shows IC₅₀ values for treatment with metformin or IM-156 of different GBM subtypes.

FIGS. 23A-D show that IM-156 exhibits higher activity in mitochondrial and F3T3-positive GBM PDOs compared with glycolytic/plurimetabolic GBM PDOs. FIG. 23A shows a summary of IM-156 activity at two concentrations in MTC, GPM, and F3T3 fusion GBM PDOs FIG. 23B shows activity of metformin treatment in three GBM PDOs. FIG. 23C shows activity of IM-156 at a concentration of 15 μM in three GBM PDOs. FIG. 23D shows activity of IM-156 at a concentration of 45 μM in three GBM PDOs.

### DETAILED DESCRIPTION OF THE INVENTION

The patent and scientific literature referred to herein establishes knowledge that is available to those skilled in the art. The issued patents, applications, and other publications that are cited herein are hereby incorporated by reference to the same extent as if each was specifically and individually indicated to be incorporated by reference.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

The terms “animal,” “subject” and “patient” as used herein includes all members of the animal kingdom including, but not limited to, mammals, animals (e.g., cats, dogs, horses, swine, etc.) and humans.

In certain aspects the subject matter described herein provides a method of treating glioblastoma (GBM) in a subject in need thereof, the method comprising: providing a GBM sample from the subject; determining a GBM subtype for the GBM sample; and administering to the subject a pharmaceutical composition, wherein the pharmaceutical composition modifies activity of one or more functional pathway associated with the GBM subtype.

In some embodiments, the GBM is IDH wild-type GBM. In some embodiments, the GBM subtype is a neurodevelopmental subtype. In some embodiments, the GBM subtype is neuronal (NEU). In some embodiments, the GBM subtype is proliferative/progenitor (PPR). In some embodiments, the GBM subtype is a metabolic subtype. In some embodiments, the GBM subtype is mitochondrial (MTC). In some embodiments, the MTC GBM subtype harbors deletions in at least a portion of chromosome 1p36.23. In some embodiments, the deletions in at least a portion of chromosome 1p36.23 comprise a deletion of gene SLC45A1. In some embodiments, the GBM subtype is glycolytic/plurimetabolic (GPM). In some embodiments, the GBM subtype comprises an FGFR3-TACC3 gene fusion.

In some embodiments, the pharmaceutical composition is an inhibitor of mitochondrial metabolism. In some embodiments, the pharmaceutical composition is an inhibitor of mitochondrial activity. In some embodiments, the pharmaceutical composition is an inhibitor of mitochondrial respiration. In some embodiments, the pharmaceutical composition is an OXPHOS inhibitor. In some embodiments, the pharmaceutical composition is IM-156. In some embodiments, the pharmaceutical composition is an inhibitor of mitochondrial complex I. In some embodiments, the pharmaceutical composition is metformin. In some embodiments, the pharmaceutical composition is IACS-010759. In some embodiments, the pharmaceutical composition is tigecycline. In some embodiments, the pharmaceutical composition is an inhibitor of mitochondrial protein translation. In some embodiments, the pharmaceutical composition is menadione. In some embodiments, the pharmaceutical composition is an inducer of mitochondrial ROS or apoptosis.

In some embodiments, the determining comprises a single cell RNA-seq analysis of the sample. In some embodiments, the determining comprises a scBiPaD analysis of the sample. In some embodiments, the determining comprises defining cluster-specific ranked-lists. In some embodiments, the determining comprises consensus clustering analysis of cell subpopulations in the sample.

In some embodiments, the determining comprises: generating a gene signature of the sample; comparing the gene signature to one or more gene signatures of GBM samples with known subtype; making a determination of the GBM subtype based on matching the gene signature to one or more known gene signature. In some embodiments, the determining further comprises a genomic analysis, a transcriptomic analysis, a DNA methylation analysis, a microRNA analysis, or a proteomics analysis of the sample.

In certain aspects the subject matter described herein provides a method of a determining clinical outcome in a subject having glioblastoma (GBM), the method comprising: providing a GBM sample from the subject; determining the a GBM subtype for the GBM sample; and providing a clinical outcome based on the GBM subtype.

In some embodiments, the GBM is IDH wild-type GBM. In some embodiments, the GBM subtype is a neurodevelopmental subtype. In some embodiments, the GBM subtype is neuronal (NEU). In some embodiments, the GBM subtype is proliferative/progenitor (PPR). In some embodiments, the GBM subtype is a metabolic subtype. In some embodiments, the GBM subtype is mitochondrial (MTC). In some embodiments, the MTC GBM subtype harbors deletions in at least a portion of chromosome 1p36.23. In some embodiments, the deletions in at least a portion of chromosome 1p36.23 comprise a deletion of gene SLC45A1. In some embodiments, the GBM subtype is glycolytic/plurimetabolic (GPM).

In some embodiments, the determining comprises a single cell RNA-seq analysis of the sample. In some embodiments, the determining comprises a scBiPaD analysis of the sample. In some embodiments, the determining comprises defining cluster-specific ranked-lists. In some embodiments, the determining comprises consensus clustering analysis of cell subpopulations in the sample.

In some embodiments, the determining comprises: generating a gene signature of the sample; comparing the gene signature to one or more gene signatures of GBM samples with known subtype; making a determination of the GBM subtype based on matching the gene signature to one or more known gene signature. In some embodiments, the determining further comprises a genomic analysis, a transcriptomic analysis, a DNA methylation analysis, a microRNA analysis, or a proteomics analysis of the sample.

Most cancers are characterized by various tumor subtypes with distinct clinical outcomes. While transcriptomic analysis has become an important tool for determining prognosis and therapeutic response in cancer patients, current methods are limited in their ability to classify diverse, aggressive cancers such as GBM. Specific tumor subtypes are often associated with differences in tumor metabolism, progression, and responsivity to given treatments (Fulda S, Galluzzi L, Kroemer G. Targeting mitochondria for cancer therapy. Nat. Rev. Drug Disc. 2010 May; 9: pp. 447-464). Transcriptomic analyses are important approaches for classification of tumors into molecular subtypes with distinct clinical outcomes and therapeutic responses (Cieślik M, Chinnaiyan A M. Cancer transcriptome profiling at the juncture of clinical translation. Nat. Rev. Genet. 2018 Feb; 19(2): pp. 93-109). However, for certain tumors such as GBM, classification via transcriptomics has failed to indicate prognosis and pharmacological vulnerability (Verhaak R G, Hoadley K A, Purdom E, Wang V, Qi Y, Wilkerson MD, Miller C R, et al; Cancer Genome Atlas Research Network. Integrated genomic analysis identifies clinically relevant subtypes of glioblastoma characterized by abnormalities in PDGFRA, IDH1, EGFR, and NFl. Cancer Cell. 2010 Jan. 19; 17(1): pp. 98-110; Wang Q, Hu B, Hu X, Kim H, Squatrito M, Scarpace L, deCarvalho AC, Lyu S, Li P, Li Y, Barthel F, et al. Tumor evolution of glioma-intrinsic gene expression subtypes associates with immunological changes in the microenvironment. Cancer Cell. 2017 Jul. 10; 32(1): pp. 42-56.e6.). The integration of multi-omics would reveal additional biomarkers of tumors and would enhance accuracy of diagnostics and prognostics in several different cancers.

In some embodiments, the subject matter described herein relates to a pipeline that seamlessly integrates multi-omics for unbiased and accurate classification of individual glioma cells and bulk tumors. In some embodiments, a computational approach was used to identify four functional states (proliferative, neuronal, mitochondrial, and glycolytic/plurimetabolic) from single cell RNA-sequencing data of high-grade gliomas. In some embodiments, combined with multi-omics analyses, this approach revealed that mitochondrial GBM is significantly associated with deletion of the glucose-proton symporter SLC45A1. In some embodiments, mitochondrial GBM has demonstrated vulnerability to inhibitors of oxidative phosphorylation. In some embodiments, mitochondrial GBM has the most optimal clinical outcome among GBM subtypes. In some embodiments, this functional pathway-based classification of tumors enables precision targeting of cancer metabolism, significantly improving diagnoses, prognoses, and treatment strategies in a personalized manner.

### Functional States

In some embodiments, the subject matter disclosed herein relates to the development of a computational approach for the unbiased identification of the core functional pathways that optimally classify both individual glioma cells and bulk tumors. In some embodiments, the subject matter described herein relates to using single cell RNA-sequencing data from high-grade gliomas to uncover four functional states that exist along two evolutionary axes. In some embodiments, 36 high-grade gliomas were used in a single cell RNA-sequencing analysis. In some embodiments, one evolutionary axis is a metabolic axis. In some embodiments, one evolutionary axis is a neurodevelopmental axis. In some embodiments, the metabolic axis includes a mitochondrial functional state. In some embodiments, the metabolic axis includes a glycolytic/pluri-metabolic functional state. In some embodiments, the neurodevelopmental axis includes a proliferative/progenitor functional state. In some embodiments, the neurodevelopmental axis includes neuronal functional states. In some embodiments, the activation of the same set of biological pathways independently stratifies primary GBM into four functional subtypes. In some embodiments, the mitochondrial subgroup is associated with the most favorable clinical outcome.

In some embodiments, the subject matter described herein relates to integrating genomic, transcriptomic, DNA methylation, microRNA and proteomics analysis to reveal that mitochondrial GBM is enriched with coherent gain-of-function of mitochondrial genes and loss-of-function alterations targeting glycolysis and alternative metabolic programs, suggesting that this subgroup may fail to produce compensatory metabolism. In some embodiments, mitochondrial GBM relies exclusively on oxidative phosphorylation for energy production whereas the glycolytic/plurimetabolic subtype is sustained by concurrent activation of multiple metabolic fluxes including aerobic glycolysis, amino acid consumption and lipid synthesis and storage. In some embodiments, deletion of SLC45A1, a gene coding for a glucose-H+ symporter on chromosome 1p36.23, is the truncal genetic alteration most significantly associated with mitochondrial GBM. In some embodiments, reintroduction of SLC45A1 in mitochondrial GBM cells harboring SLC45A1 gene deletion induces cytoplasmic acidification, loss of cell fitness and growth arrest. In some embodiments, the strict dependency of mitochondrial GBM on mitochondrial respiration is associated with excessive generation of reactive oxygen species and unique sensitivity to inhibitors of oxidative phosphorylation. In some embodiments, the subject matter disclosed herein relates toa functional classification of GBM that informs clinical outcome and identifies patients who are more likely to benefit from therapies targeting metabolic vulnerabilities. In some embodiments, the pathway-based classification of GBM informs survival and enables precision targeting of cancer metabolism.

### Method of Treatment

In some embodiments, the subject matter described herein relates to treating patients suffering with cancer. In some embodiments, the subject matter described herein relates to treating patients suffering with GBM. In some embodiments, the patients are in an initial stage of the disease. In some embodiments, the patients are in an advanced stage of disease progression. In some embodiments, a biopsy sample is obtained from a patient. In some embodiments, at least one biopsy sample is obtained from the patient's brain. In some embodiments, at least one biopsy sample is obtained from the patient's brain tumor using excess tissue that would be normally discarded. In some embodiments, the biopsy is obtained by any method known in the art. In some embodiments, cell metabolism is determined using a scan. In some embodiments, the patients are subjected to one of more brain scans. In some embodiments, the scan is a positron emission tomography (PET) scan, a magnetic resonance imaging (MM) scan, computerized tomography (CT) scan. In some embodiments, the scan includes imaging for cellular respiration or ROS levels in in the patient's body.

In some embodiments, the method of treatment includes performing a computational analysis on the biopsy obtained from the patients for the identification of the core functional pathways. In some embodiments, this computational analysis classifies tumors into different subtypes. In some embodiments, the computational analysis is a single cell RNA-seq approach. In some embodiments, the computational analysis is the scBiPaD method as described below. In some embodiments, the computational analysis is integrated with genomic, transcriptomic, DNA methylation, microRNA, and/or proteomics analysis.

In some embodiments, the method of treatment comprises generating one or more gene signatures of the patient's tumor sample to classify the tumor subtype. In some embodiments, the gene signatures are generated using one of more computational analyses. In some embodiments, the gene signatures are generated using single cell RNA-seq analysis. In some embodiments, the gene signatures are generated using scBiPaD analysis. In some embodiments, the gene signatures are generated using any of the computational methods described here, or a combination thereof. In some embodiments, the generated gene signatures are compared to the gene signatures of previously characterized tumors. In some embodiments, the gene signatures of previously characterized tumors have been previously characterized and validated using and of the computational methods described herein or a combination thereof. In some embodiments, the gene signatures are generated by using a Mann-Whitney-Wilcoxon (MWW) test to derive ranked lists of genes differentially expressed in each of the tumor subtypes compared to the others. In some embodiment, for each tumor subtype the final gene signature includes the first 50 highest scoring genes in the ranked list. In some embodiments, these gene signatures are used to calculate the enrichment of each functional tumor subtype for each bulk tumor. In some embodiments, the enrichment is expressed as a normalized enrichment score (NES). In some embodiments, the simplicity score for each individual tumor is computed as the difference between the highest NES (dominant subtype) and the mean of the other subtypes (non-dominant). In some embodiments, the simplicity score represents the subtype activation: higher scores indicate lower transcriptional complexity and lower scores multi-subtype activation. In some embodiments the tumor is a GBM.

In some embodiments, the treatment includes determining the subtype of GBM in a patient suffering with GBM. In some embodiments, the GBM is IDH wild-type GBM. In some embodiments, the is IDH wild-type GBM is the most aggressive type of GBM. In some embodiments, the GBM is a metabolic GBM. In some embodiments, the GBM is a neurodevelopmental GBM. In some embodiments, the GBM is mitochondrial GBM. In some embodiments, the GBM is a glycolytic/pluri-metabolic GBM. In some embodiments, the GBM is a proliferative/progenitor GBM. In some embodiments, the GBM is neuronal GBM. In some embodiments, the GBM lacks at least a portion of chromosome 1p36.23. In some embodiments, the lacking portion of chromosome 1p36.23 includes the SLC45A1 gene, encoding for a glucose-proton (H+) symporter. In some embodiments, mitochondrial GBM is associated with deletion of the SLC45A1 gene. In some embodiments, the mitochondrial GBM lacks a functional SLC45A1 glucose-proton (H+) symporter. In some embodiments, the GBM subtype carries a FGFR3-TACC3 gene fusion.

In some embodiments, the subject matter disclosed herein relates to administering a pharmaceutical composition to a patient suffering with cancer based on the cancer subtype. In some embodiments, the subject matter disclosed herein relates to administering a pharmaceutical composition to a patient suffering with GBM based on the GBM subtype. In some embodiments, the core functional pathways specific to the GBM subtype are altered by the pharmaceutical composition. In some embodiments, the core functional pathways of the GBM subtype render the GBM subtype susceptible to the pharmaceutical composition.

In some embodiments, the pharmaceutical composition is an inhibitor of mitochondrial metabolism. In some embodiments, the pharmaceutical composition is an inhibitor of mitochondrial activity. In some embodiments, the pharmaceutical composition is an inhibitor of mitochondrial respiration. In some embodiments, the pharmaceutical composition is an OXPHOS inhibitor. In some embodiments, the pharmaceutical composition is IM-156. In some embodiments, the pharmaceutical composition administered to a patient with mitochondrial GBM is IM-156. In some embodiments, the pharmaceutical composition administered to a patient with glycolytic/plurimetabolic GBM is IM-156. In some embodiments, the IM-156 dosage administered to a patient with glycolytic/plurimetabolic GBM is higher that the dosage administered to a patient with mitochondrial GBM. In some embodiments, the pharmaceutical composition is an inhibitor of mitochondrial complex I. In some embodiments, the pharmaceutical composition is metformin. In some embodiments, the pharmaceutical composition is IACS-010759. In some embodiments, the pharmaceutical composition is tigecycline. In some embodiments, the pharmaceutical composition is an inhibitor of mitochondrial protein translation. In some embodiments, the pharmaceutical composition is menadione. In some embodiments, the pharmaceutical composition is an inducer of mitochondrial ROS or apoptosis. In some embodiments, the pharmaceutical composition is an FGFR inhibitor. In some embodiments, the pharmaceutical composition is a small molecule. In some embodiments, the pharmaceutical composition is an antibody or a cocktail of antibodies. In some embodiments, the pharmaceutical composition is a bispecific antibody or a cocktail of bispecific antibodies. In some embodiments, the pharmaceutical composition is a siRNA. In some embodiments, the pharmaceutical composition is a CRISPR/CAS system. In some embodiments, the pharmaceutical composition is a CAR-T therapy. In some embodiments, the pharmaceutical composition includes any molecule known in the art to interfere with gene or protein expression.

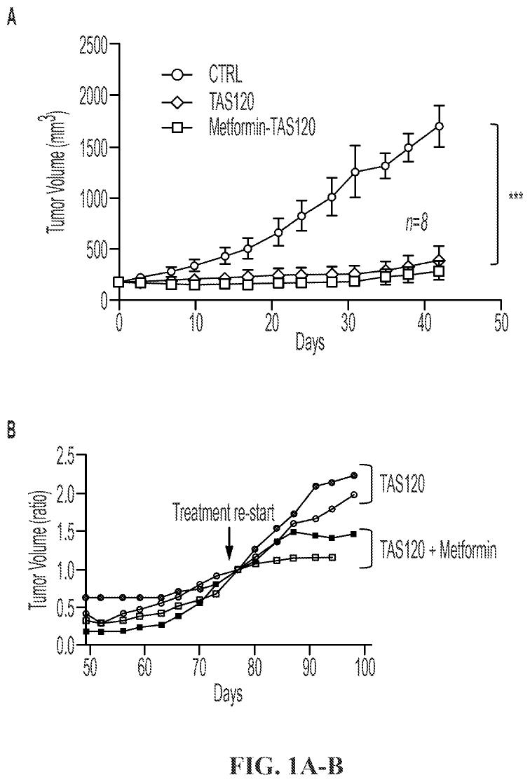

In some embodiments, the subject matter disclosed herein relates to the administration of a combination of therapy. In some embodiments, the combination of therapy is a combination of anti-mitochondrial cancer therapy with genetic targeting. In some embodiments, the combination of therapy is a combination of anti-mitochondrial GBM therapies with genetic targeting. As shown in FIGS. 1A-B, there is a synergistic effect in FGFR3-TACC3-positive tumors treated with FGFR inhibitors and OXPHOS inhibitors. In some embodiments, any of the therapies disclosed herein can be used in a combination with any other therapy disclosed herein or known in the art. In some embodiments, the subject matter disclosed herein relates to overcoming drug resistance in cancer treatment. In some embodiments, the subject matter disclosed herein relates to a combinatorial treatment of cancer patients harboring FGFR-TACC protein fusions with mitochondrial inhibitors and FGFR-kinase inhibitors.

In some embodiments, the subject matter disclosed herein relates to a method of screening patients with GBM for a deletion in chromosome 1p36.23. In some embodiments, the subject matter disclosed herein relates to a method of screening patients with GBM for a deletion of the SLC45A 1 gene. In some embodiments, the method of screening involves determining the presence or absence of the SLC45A1 gene in a sample of the patient's tumor. In some embodiments, the determining is performed using a single-cell RNA-seq analysis. In some embodiments, the determining is performed using a scBiPaD method. In some embodiments, the subject matter described herein relates to treating patients suffering with GBM with a deletion of the SLC45A/gene. In some embodiments, patients with GBM with a deletion of the SLC45A/gene are treated with a pharmaceutical composition, which is an inhibitor of mitochondrial metabolism. In some embodiments, the pharmaceutical composition is an inhibitor of mitochondrial activity. In some embodiments, the pharmaceutical composition is an inhibitor of mitochondrial respiration. In some embodiments, the pharmaceutical composition is an oxidative phosphorylation (OXPHOS) inhibitor. In some embodiments, the pharmaceutical composition is IM-156. In some embodiments, the pharmaceutical composition is an inhibitor of mitochondrial complex I. In some embodiments, the pharmaceutical composition is metformin. In some embodiments, the pharmaceutical composition is IACS-010759. In some embodiments, the pharmaceutical composition is an inhibitor of mitochondrial protein translation. In some embodiments, the pharmaceutical composition is an inducer of mitochondrial ROS or apoptosis.

In some embodiments, the subject matter disclosed herein relates to a method of screening patients with GBM for a deletion of the ENO1 gene. In some embodiments, the method of screening involves determining the presence or absence of the ENO1 gene in a sample of the patient's tumor. In some embodiments, the determining is performed using a single-cell RNA-seq analysis. In some embodiments, the determining is performed using a scBiPaD method. In some embodiments, the subject matter described herein relates to treating patients suffering with GBM with a deletion of the ENO1 gene. In some embodiments, patients with GBM with a deletion of the ENO1 gene are treated with a pharmaceutical composition, which is an inhibitor of mitochondrial metabolism. In some embodiments, the pharmaceutical composition is an inhibitor of mitochondrial activity. In some embodiments, the pharmaceutical composition is an inhibitor of mitochondrial respiration. In some embodiments, the pharmaceutical composition is an oxidative phosphorylation (OXPHOS) inhibitor. In some embodiments, the pharmaceutical composition is IM-156. In some embodiments, the pharmaceutical composition is an inhibitor of mitochondrial complex I. In some embodiments, the pharmaceutical composition is metformin. In some embodiments, the pharmaceutical composition is IACS-010759. In some embodiments, the pharmaceutical composition is an inhibitor of mitochondrial protein translation. In some embodiments, the pharmaceutical composition is an inducer of mitochondrial ROS or apoptosis.

### Identifying Mitochondrial Subtypes Across All Human Tumor Types

In some embodiments, the subject matter disclosed herein relates to identifying mitochondrial subtypes across all human tumor types. In some embodiments, the subject matter disclosed herein relates to anti-mitochondrial therapeutic targeting of mitochondrial subtypes across all tumor types. In some embodiments, the subject matter disclosed herein relates to targeting mitochondrial subtypes identified with the approach disclosed herein, which may result in generally applicable precision therapeutics of cancer metabolism.

In some embodiments, identifying mitochondrial subtypes across all human tumor types comprises preparing cDNA libraries for analysis. In some embodiments, RNA is isolated from a tumor sample. In some embodiments, the tumor sample is removed at surgery. In some embodiments, the tumor sample is in excess of the sample needed for all diagnostic analyses and would be discarded. In some embodiments, cDNA is synthesized from the isolated RNA. In some embodiments, one or more libraries of the synthesized cDNA are prepared. In some embodiments, the libraries are sequenced. In some embodiments, one or more data sets are generated from the sequenced libraries. In some embodiments, the one or more data sets generated from the sequenced libraries are analyzed to classify the tumor. In some embodiments, the isolated RNA is sequenced without a cDNA synthesis step. In some embodiments, one or more data sets are generated from the sequenced RNA. In some embodiments, the one or more data sets generated from the sequenced RNA are analyzed to classify the tumor.

### Single Cell RNA-Sequencing Analysis and Bulk GBM Multi-Omics—Definition of Single-Sample Pathway Activity

The MSigDB c5.bp, c5.mf, c5.cc, Hallmark and KEGG collections of gene sets, retaining only pathways composed of at least 15 genes, resulting in 5,032 gene sets were aggregated. Pathway enrichment in each individual cell was computed by adapting the Mann-Whitney-Wilcoxon Gene Set test (MWW-GST) originally developed for the analysis of unbalanced datasets. When used in comparative analysis, MWW-GST requires as input a gene set and a ranked list representing the gene-wise differential expression between the two groups. When adapted to single cell analysis (single sample MWW-GST, ssMWW-GST), to determine the relative expression of individual cells in each tumor, the expression of each gene is standardized for the expression in the cell cohort and used to generate a cell-specific ranked list. Ranked lists of single cells and pathway gene sets are used as input for ssMWW-GST. The resulting normalized enrichment score, NES, is an estimate of the probability that the expression of genes in the gene set is greater than the expression of genes outside the gene set:

$$NES = 1 - \frac{U}{mn}$$

where m is the number of genes in a gene set, n is the number of those outside the gene set,

$$U = nm + \frac{m ( m + 1 )}{2} - T ,$$

and T is the sum of the ranks of genes in the gene set. Thus, NES is a reporter of pathway activity with values near zero meaning down-regulation of the pathway and values near one indicating up-regulation of the pathway. In addition to the NES, MWW-GST generates a p-value for each pathway activity, a parameter considered for the selection of enriched pathways.

### Characterization of the Biological States of the Single Cell Subpopulations

To characterize the four clusters of cell sub-populations, the medoids of each cluster were obtained by applying the Partitioning Around Medoids (PAM) clustering algorithm (Van der Laan, M. J., Pollard, K. S. & Bryan, J. A new partitioning around medoids algorithm. J Stat Comput Sim 73, 575-584 (2003). A medoid is defined as an object that minimizes the sum distance of this object to the other objects within its cluster, thus reflecting all objects in the cluster. In the datasets used herein, the medoid is a binary vector having a value of 1 for the enriched pathways in the cell sub-population. The pathways were then used to construct Gene Ontology-guided maps using the Enrichment Map application of Cytoscape (version 3.7.2) after removing gene sets that included more than 250 genes (650 gene pathways), thus avoiding the preferential selection of very large uninformative pathways and applying the enrichment set cover algorithm (Isserlin, R., Merico, D., Voisin, V. & Bader, G. D. Enrichment Map—a Cytoscape app to visualize and explore OMICs pathway enrichment results. F1000Res 3, 141 (2014); Smoot, M. E., Ono, K., Ruscheinski, J., Wang, P. L. & Ideker, T. Cytoscape 2.8: new features for data integration and network visualization. Bioinformatics 27, 431-432 (2011); Stoney, R. A., Schwartz, J. M., Robertson, D. L. & Nenadic, G. Using set theory to reduce redundancy in pathway sets. BMC Bioinformatics 19, 386 (2018)). To extract the biological functions that specifically drive each cluster, pathways with significant differential activity were selected in a specific cluster compared to the others using two-sided MWW test (effect size >0.3 and FDR<0.0001). Cluster assignments by scBiPaD were further confirmed by applying the entire workflow independently to each of the three single cell datasets. This analysis produced 4 clusters for each dataset having very similar enrichments as the combined analysis. When the sub-populations from the individual datasets were combined, ˜95% concordance of cell sub-population identity with the analysis performed on the aggregated datasets was obtained.

### Definition of Single Cell Gene Meta-Signature, Subtype Classification and State Coexistence

For each cell sub-population cluster, a meta-signature was defined based on the average gene MWW-scores across cell sub-populations of the same cluster using the three single cells datasets combined. Each meta-signature consisted of the 50 highest scoring genes. Glioma cells were then assigned to each individual subtype on the basis of the highest significant score using ssMWW-GST [log it(NES)>0 and FDR<0.01]. ssMWW-GST was also used to classify cells according to lineage states and the correlation between pathway-based functional states and lineages states was examined by ξ² test (Frattini, V., et al. A metabolic function of FGFR3-TACC3 gene fusions in cancer. Nature 553, 222-227 (2018); Caruso, F. P., et al. A MAP of tumor-host interactions in glioma at single cell resolution. GigaScience 9, (2020); D'Angelo, F., et al. The molecular landscape of glioma in patients with Neurofibromatosis 1. Nat Med 25, 176-187 (2019); Zhang, J., et al. The combination of neoantigen quality and T lymphocyte infiltrates identifies glioblastomas with the longest survival. Commun Biol 2, 135 (2019)). Analysis of the co-existence of cell states within individual tumors was performed using Spearman's correlation and classical multidimensional scaling (CMDS) of the distribution of GBM states per tumor with k-NN (k=2) tumor clustering.

### Identification of Glioma Subtypes in the TCGA IDH Wild Type Glioblastoma Cohort

The GBM dataset from The Cancer Genome Atlas (TCGA) collection profiled with Agilent chip G4502A was used. The matrix of the raw data was quantile normalized. The gene expression data matrix includes 534 samples and 17,814 genes. To determine the clinical relevance of the biological subtypes, those pathways capable of segregating patients according to survival were identified. Survival data are available for 527 IDH wild type GBMs from TCGA and were downloaded using TCGAbiolinks R/Bioconductor package (Colaprico, A., et al. TCGAbiolinks: an R/Bioconductor package for integrative analysis of TCGA data. Nucleic Acids Res 44, e71 (2016)). Pathway enrichment in each individual tumor was computed by ssMWW- GST. For each pathway, 3 groups of patients were defined: (i) high activity group: patients whose tumor had activation of the pathway [log it(NES)>0 and FDR<0.01]; (ii) low-activity group: patients whose tumor exhibited inactivation of the pathway [log it(NES)<0 and FDR<0.01]; (iii) neutral activity group: patients whose tumor lacked activation or inactivation of the pathway (FDR>0.01). Survival was evaluated by the log-rank test: (i) high versus low activity group; (ii) high versus low versus neutral activity group. The positive or negative correlation with outcome was established using the Cox's proportional hazards regression model of hazard ratio (HR). For further analysis, those pathways that resulted in a significant survival difference in any comparison (p<0.05) were selected, totalizing 192 pathways. Given the i-th and j-th tumors and a pathway p from the 192 survival-associated, we defined gᵢⱼ(p)=1 if both tumors belonged to the same pathway activity group, 0 otherwise. Finally, a dissimilarity matrix (C) was constructed as

$$C ( i , j ) = 1 - \frac{1}{192} {\sum }_{p = 1}^{192} {g}_{ij} ( p ) .$$

The distance induced by matrix C was then used to cluster samples using consensus clustering (Ward linkage method, 10,000 resampling steps with 70% of samples). Calinski and Harabasz criterion was then used to derive K=5 as the best number of clusters.

### Characterization of GBM IDH Wild Type Tumors in Functional Subtypes

Differential activated pathways were identified among the 192 pathways associated with survival using two-sided MWW test (effect size >0.3 and FDR<0.01). This analysis produced 126 pathways. The pathways were then examined, among the entire collection of 5,032, that were differential activated between the four GBM sub-groups and identified 2,792 pathways. Finally, the Kruskal-Wallis H test was used to select genes that showed any difference in expression levels between the four subtypes (FDR<0.01) and the post hoc Nemenyi's test for multiple comparison correction to identify genes with significant differential expression in one subtype compared with the others [log 2(FC)>0.5 and FDR <0.01] (Hollander, M., Wolfe, D. A. & Chicken, E. Nonparametric statistical methods, (John Wiley & Sons, Inc., Hoboken, New Jersey, 2014)).

### Definition of Bulk GBM Gene Signatures and Simplicity Score

The MWW test was used to derive ranked lists of genes differentially expressed in each of the subtypes compared to the others. For each subtype the final gene signature included the first 50 highest scoring genes in the ranked list. These gene signatures were used to calculate the enrichment of each functional GBM subtype (normalized enrichment score, NES) for each bulk tumor. The simplicity score for each individual tumor was then computed as the difference between the highest NES (dominant subtype) and the mean of the other subtypes (non-dominant). The simplicity score represents the subtype activation: higher scores indicate lower transcriptional complexity and lower scores multi-subtype activation.

### Supervised Classification of Unclassified TCGA GBM

To assign state subtype memberships to the 230 tumors from the unclassified (black) cluster, the 304 GBM initially classified as the training set of a k-NN classifier (k=3) were used. The classifier feature set included the expression of the 100 highest scoring genes in the ranked list of each subtype. Twenty-eight tumors with conditional probability to subtype memberships <0.6 remained unclassified and were excluded from subsequent analyses. The samples classified by k-NN were integrated with 304 samples obtained from consensus clustering and used in the analysis of genetic alterations associated with GBM subgroups.

In some embodiments, the subject matter described herein relates to a method of determining clinical outcome in a subject having glioblastoma (GBM), the method comprising: providing a GBM sample from the subject; determining a GBM subtype of the GBM sample via a pathway-based classifier approach; and determining the clinical outcome based on the GBM subtype. In some embodiments, the GBM is IDH wild-type GBM. In some embodiments, GBM subtype is characterized by attributes of development. In some embodiments, the GBM subtype is neuronal (NEU). In some embodiments, the GBM subtype is proliferative/progenitor (PPR). In some embodiments, the GBM subtype is characterized by attributes of metabolism. In some embodiments, the GBM subtype is mitochondrial (MTC). In some embodiments, the MTC GBM subtype harbors deletions of chromosome 1p36.23. In some embodiments, the deletions of chromosome 1p36.23 comprise a deletion of a SLC45A1 gene, encoding for a glucose-proton (H+) symporter. In some embodiments, the GBM subtype is glycolytic/plurimetabolic (GPM). In some embodiments, the pathway-based classifier approach comprises scRNA-seq analyses of a GBM sample. In some embodiments, the pathway-based classifier approach comprises a scBiPad analyses of a GBM sample. In some embodiments, the analysis is a single cell analysis.

### Single Cell Biological Pathway Deconvolution (scBiPaD) Computational Method

In some embodiments, the treatment includes performing a pathway-based classification analysis on the biopsy sample. In some embodiments, the subject matter disclosed herein relates to an analysis of core functional pathways in a biopsy sample from a patient's GBM. In some embodiments the analysis is a single cell RNA-seq analysis. In some embodiments, the analysis is a scBiPad analysis. In some embodiments, the subject matter described herein relates to a computational approach for the identification of the core functional pathways in cells. In some embodiments, the computational approach can be used classify tumors based on core functional pathways in the one or more of the tumor cells. In some embodiments, the tumor cells are one or more GBM cells. In some embodiments, the tumor cells are one or more of breast cancer cells, lung cancer cells, prostate cancer cells, colon and rectum cancer cells, melanoma cells, bladder cancer cells, kidney cancer cells, pancreatic cancer cells, thyroid cancer cells, or liver cancer cells.

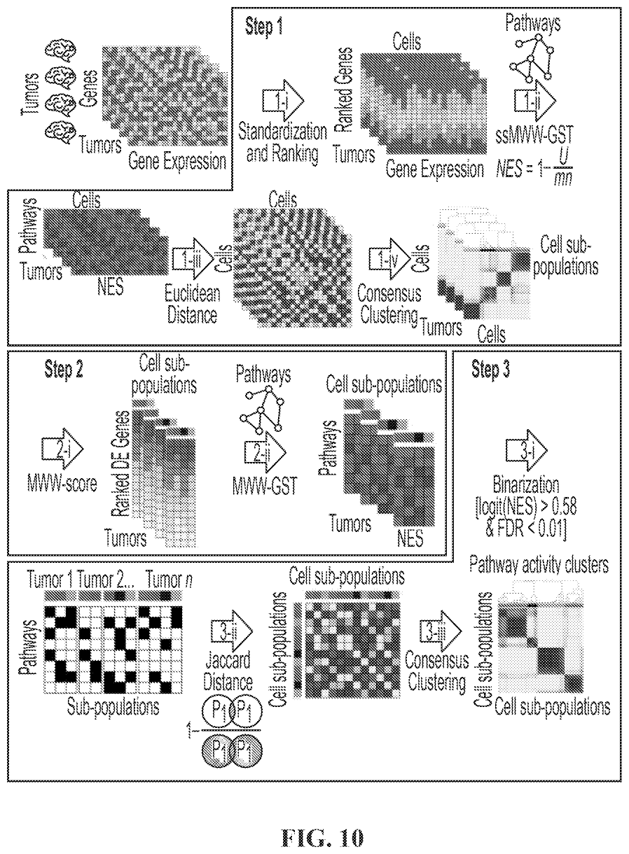

In some embodiments the subject matter described herein relates to the development of a computational approach designed as single cell Biological Pathway Deconvolution (scBiPaD) to identify coherent functional states in single cells across multiple tumors. Cancer phenotypes classification methods based on gene-level genome-wide expression profiles fail to capture the relationships and interactions between system components of the different cellular states within a single tumor. In some embodiments, scBiPaD acquires pathway-based aggregation of gene information and incorporates gene-gene relationships. In some embodiments, scBiPaD includes the three following steps (FIG. 10): Step 1) identification of cell sub-populations in each individual tumor that share activation of similar biological functions; Step 2) determination of enriched biological pathways in each cell sub-population by defining cluster-specific ranked-lists; Step 3) identification of cell sub-populations that share coherent biological functions across multiple tumors.
Step 1-i (Standardization and Ranking): for each cell, genes were ranked after standardization for the expression of each gene across cells composing each tumor.
Step 1-ii (Pathway Activity): the activity of all the 5,032 biological pathways (NES) was calculated for each single-cell with MWW-GST using the ranked list of the individual cell. Thus, each cell was represented by a vector of 5,032 values of NES that were used to derive the tumor sample-specific activity matrix.
Step 1-iiil (Euclidean Distance): the activity matrix was used to generate the Euclidean distance matrix between every pair of cells in each tumor.
Step 1-iv (Consensus Clustering): the Euclidean distance matrix was then used to inform a consensus clustering between cells of each tumor (10,000 random samplings using 70% of the cells and the Ward linkage method). For each tumor, the optimal number of clusters was determined using the Calinski and Harabasz criterion (Calinski, T. A Dendrite Method for Cluster Analysis. Biometrics 24, 207-& (1968). Only cells having a silhouette score >0.5 and clusters composed of at least 10 cells were retained for further analysis. The application of this approach to each of the 36 tumors from three single cell datasets revealed 94 sub-populations, with a number of sub-populations in each tumor ranging from 2 to 5, and 91% of cells retained after the filtering step. All retained clusters were further inspected in order to elucidate their biological significance.
Step 2i (Gene Scoring): to define the biological pathways enriched in sub-populations of individual tumors composing distinct clusters, a cluster-specific ranked-list of genes was derived comparing the expression profiles of the cells in the cluster with all other cells in the same tumor using the Mann-Whitney-Wilcoxon test, defining a score for each gene j as

$$MWW - score = \frac{{U}_{j}}{nm} ,$$

where U! is the MWW test statistic for the j-th gene, n is the number of cells in the cluster, and m is the number of cells in the other clusters.
Step 2-ii (Pathway Activity): the cluster-specific ranked lists were used to identify pathways activated in each cell sub-population using MWW-GST as in Step 1-ii.
Step 3-i (Binarization): to identify biologically coherent cell sub-populations across multiple tumors from the combined datasets, each cell subpopulation was represented with a binary vector of length 5,032, with 1 indicating the enriched biological pathways [log it(NES)>0.58 and FDR<0.01].
Step 3-ii (Jaccard Distance): the degree of overlap of enrichment between sub-populations was then computed by using the Jaccard coefficient of similarity (index) defined as:

$$J ( {p}_{{it}^{′}} \text{?} {p}_{{jt}^{″}} ) = \frac{\vert {p}_{{it}^{′}} ⋂ {p}_{{jt}^{″}} \vert}{\vert {p}_{{it}^{′}} ⋃ {p}_{{jt}^{″}} \text{?}}$$

$$\text{?} \text{indicates text missing or illegible when filed}$$

where pᵢₜ′ and pⱼₜ′ are the enriched biological pathways of sub-population i of tumor t′ and sub-population j in tumor t″. The Jaccard index is a measure of similarity between two sets, with 0 indicating no overlap and 1 indicating complete overlap. Then, the Jaccard distance is derived, defined as 1-(Jaccard index).
Step 3-iii (Consensus Clustering): the Jaccard distance was used to cluster cell sub-populations using consensus clustering (Ward linkage method, 10,000 resampling steps with 70% of sub-populations). Calinski and Harabasz criterion was used to derive K=4 as the optimal number of clusters.

In some embodiments, the subject matter described herein relates to a method of identifying functional states in single cells of more than one tumor, the method comprising: identifying cell sub-populations in each individual tumor that share activation of similar biological functions; determining of enriched biological pathways in each cell sub-population by defining cluster-specific ranked-lists; identifying cell sub-populations that share coherent biological functions across the more than one tumor. In some embodiments, the tumors are of the same type of tumor. In some embodiments, identifying cell sub-populations in each individual tumor that share activation of similar biological functions comprises: standardizing the expression level of each gene across cells composing each tumor of the more than one tumors followed by ranking the genes by expression level; calculating the activity of biological pathways for each cell; calculating the Euclidean distance matrix between every pair of cells in each tumor of the more than one tumors; performing consensus clustering between cells of each tumor of the more than one tumors. In some embodiments, the determining of enriched biological pathways in each cell sub-population by defining cluster-specific ranked-lists comprises: deriving a cluster-specific ranked-list of genes comparing the expression profiles of the cells in the cluster with all other cells in the same tumor and using the cluster-specific ranked lists to identify pathways activated in each cell sub-population. In some embodiments the deriving comprises using the Mann-Whitney-Wilcoxon test. In some embodiments, the score for each gene j is defined as

$$MWW - score = \frac{{U}_{j}}{nm} .$$

In some embodiments, U! is the MWW test statistic for the j-th gene. In some embodiments, n is the number of cells in the cluster. In some embodiments m is the number of cells in the other clusters. In some embodiments, the cluster-specific ranked lists were used to identify pathways activated in each cell sub-population using MWW-GST. In some embodiments, the identification of cell sub-populations that share coherent biological functions across multiple tumors comprises: representing each cell subpopulation with a binary vector of length 5,032; computing the degree of overlap of enrichment between sub-populations; and clustering cell sub-populations using consensus clustering.

In some embodiments, in the binary vector, 1 indicates the enriched biological pathways [log it(NES)>0.58 and FDR<0.01]. In some embodiments, the degree of overlap of enrichment between sub-populations is computed by using the Jaccard coefficient of similarity (index) defined as

$$J ( {p}_{{it}^{′}} \text{?} {p}_{{jt}^{″}} ) = \frac{\vert {p}_{{it}^{′}} ⋂ {p}_{{jt}^{″}} \vert}{\vert {p}_{{it}^{′}} ⋃ {p}_{{jt}^{″}} \text{?}} .$$

$$\text{?} \text{indicates text missing or illegible when filed}$$

In some embodiments, pᵢₜ′ and pⱼₜ′ are the enriched biological pathways of sub-population i of tumor t′ and sub-population j in tumor t″. In some embodiments, the Jaccard index is a measure of similarity between two sets, with 0 indicating no overlap and 1 indicating complete overlap. In some embodiments, the Jaccard distance is derived, defined as 1-(Jaccard index). In some embodiments, the Jaccard distance was used to cluster cell sub-populations using consensus clustering. In some embodiments, the Calinski and Harabasz criterion was used to derive K=4 as the optimal number of clusters. In some embodiments, the computational approach described herein can be integrated with genomic, transcriptomic, DNA methylation, microRNA, and/or proteomics analysis.

### EXAMPLES

### Example 1—Pathway-Based Classification of Glioblastoma Uncovers a Mitochondrial Subtype with Therapeutic Vulnerabilities

### Abstract

The transcriptomic classification of glioblastoma (GBM) has failed to predict survival and therapeutic vulnerabilities. A computational approach for unbiased identification of core biological traits of single cells and bulk tumors uncovered four tumor cell states and GBM subtypes distributed along neurodevelopmental and metabolic axes and classified as proliferative/progenitor, neuronal, mitochondrial and glycolytic/plurimetabolic. Each subtype was enriched with biologically coherent multiomic features. Mitochondrial GBM was associated with the most favorable clinical outcome. It relied exclusively on oxidative phosphorylation for energy production, whereas the glycolytic/plurimetabolic subtype was sustained by aerobic glycolysis and amino acid and lipid metabolism. Deletion of the glucose-proton symporter SLC45A1 was the truncal alteration most significantly associated with mitochondrial GBM, and the reintroduction of SLC45A1 in mitochondrial glioma cells induced acidification and loss of fitness. Mitochondrial, but not glycolytic/plurimetabolic, GBM exhibited marked vulnerability to inhibitors of oxidative phosphorylation. The pathway-based classification of GBM informs survival and enables precision targeting of cancer metabolism.

### Introduction

Transcriptomic analyses have emerged as important approaches for the classification of tumors into molecular subtypes with distinct clinical outcome and response to therapies¹. However, for certain tumors such as GBM, the transcriptomic classification has failed to indicate prognosis and pharmacologic vulnerability^{2,3}, especially when considering the highly aggressive isocitrate dehydrogenase (IDH) wild-type group. Regarding nomenclature, GBM tumors with normal, non-mutated IDH genes referred to as “IDH wild-type” or “IDH negative” tend to behave far more aggressively. On the other hand, GBM tumors with mutations of IDH genes are referred to as “IDH-mutant” or in older literature “IDH positive”, Although mutation of IDH is seen early in gliomagenesis and is oncogenic, IDH-mutant confers a better prognosis than gliomas without the mutation (IDH wild-type). Therefore, the lack of association between biologically defined subgroups of IDH wild-type GBM and survival has hindered the discovery of the unique mechanisms that sustain tumor progression in subgroups of patients.

Recent data in single cells have shown that the transcriptomic subgroups used to classify GBM are preferentially enriched in tumor cells exhibiting distinct lineage-specific cellular states⁴. However, it remains untested whether fundamental biological activities of individual GBM cells can be used to build a classification of bulk tumors that is also clinically informative. Because pathway-based classifications of transcriptomic cancer data have shown higher stability of biological activities and better performance than gene-based classifiers⁵, we developed a computational approach to extract the core tumor cell intrinsic biological states of individual GBM cells from GBM single-cell RNA-sequencing (scRNA-seq) data^{4,6,7} and bulk tumors. The analyses converged on four stable cellular states that embody metabolic (mitochondrial and glycolytic/plurimetabolic) and neurodevelopmental (neuronal and proliferative/progenitor) attributes, and generated a new GBM classification. The mitochondrial subtype is dependent on oxidative phosphorylation (OXPHOS) and stratifies patients with a more favorable clinical outcome. Multiomics analysis revealed that the mitochondrial group of GBM contrasts with the poor-prognosis, glycolytic/plurimetabolic subgroup that is sustained by concurrent activation of multiple energy-producing programs, which confer metabolic versatility and protection from oxidative stress. The mitochondrial subgroup of GBM exhibits unique sensitivity to inhibitors of mitochondrial metabolism, thus providing insights into the selection of patients with GBM who could benefit from targeted metabolic therapies.

### Results

### Pathway-Based Analysis of Single Glioma Cells Identifies Four Cellular States Converging On Two Biological Axes

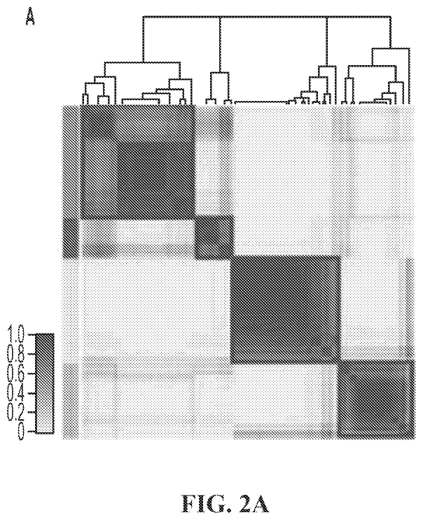

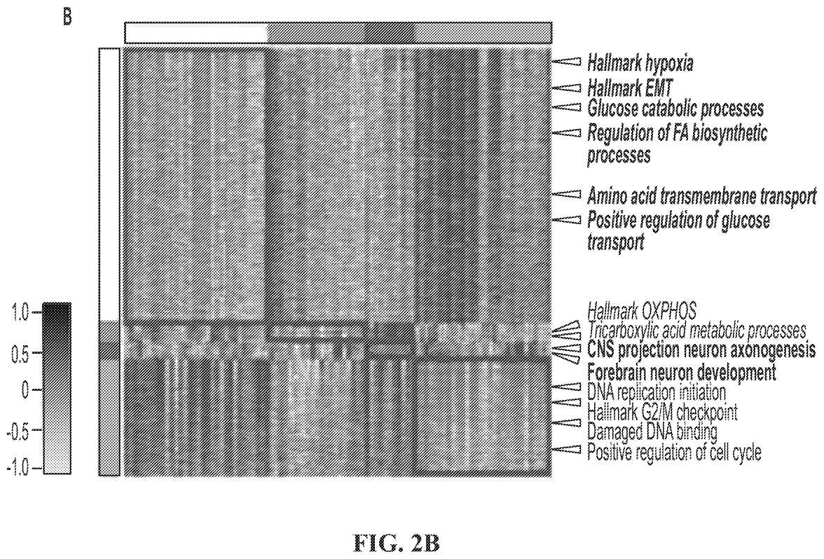

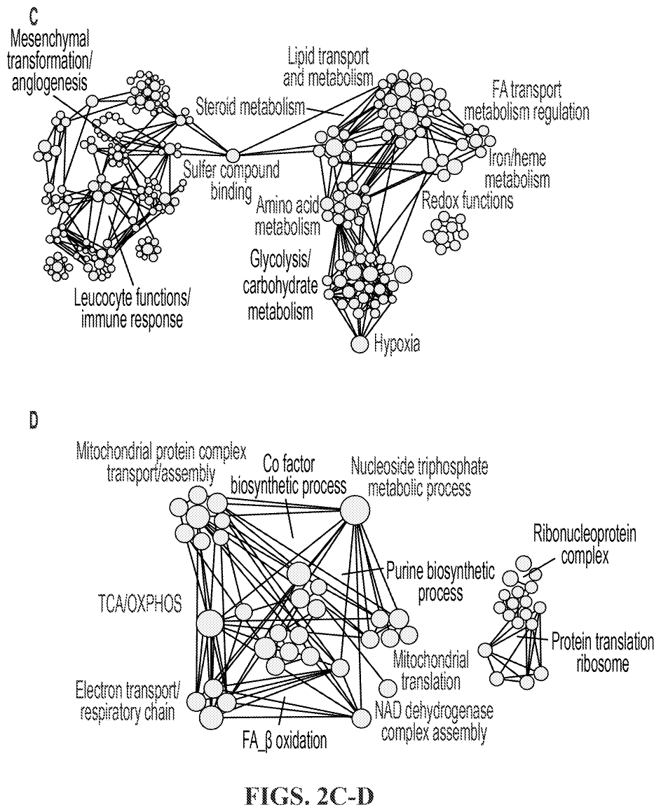

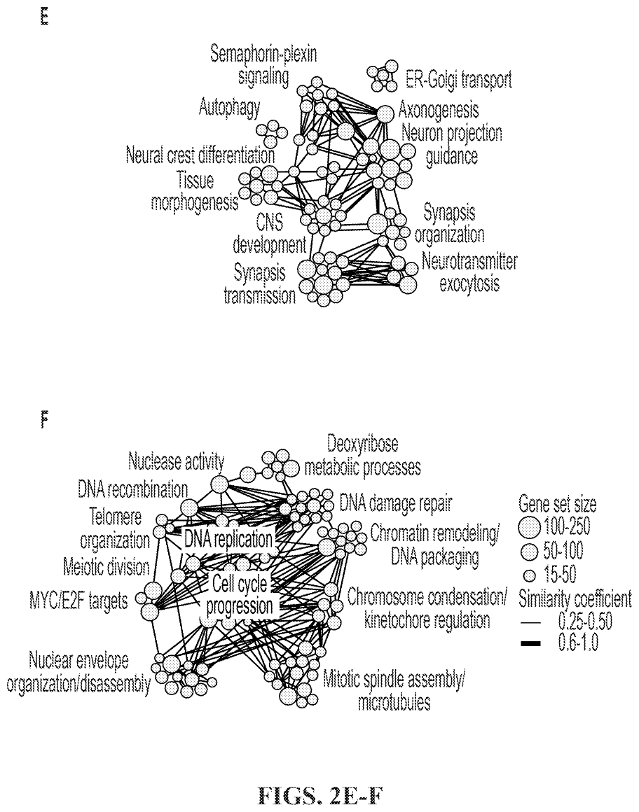

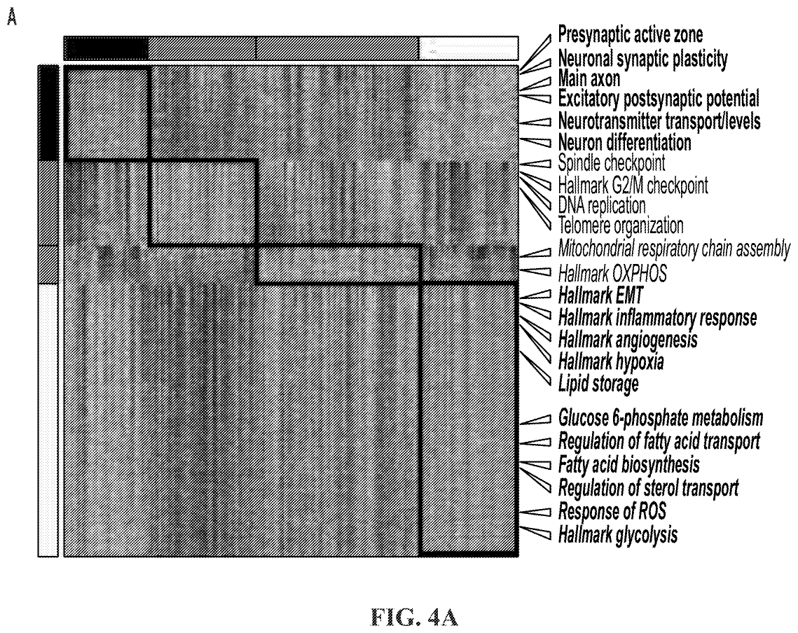

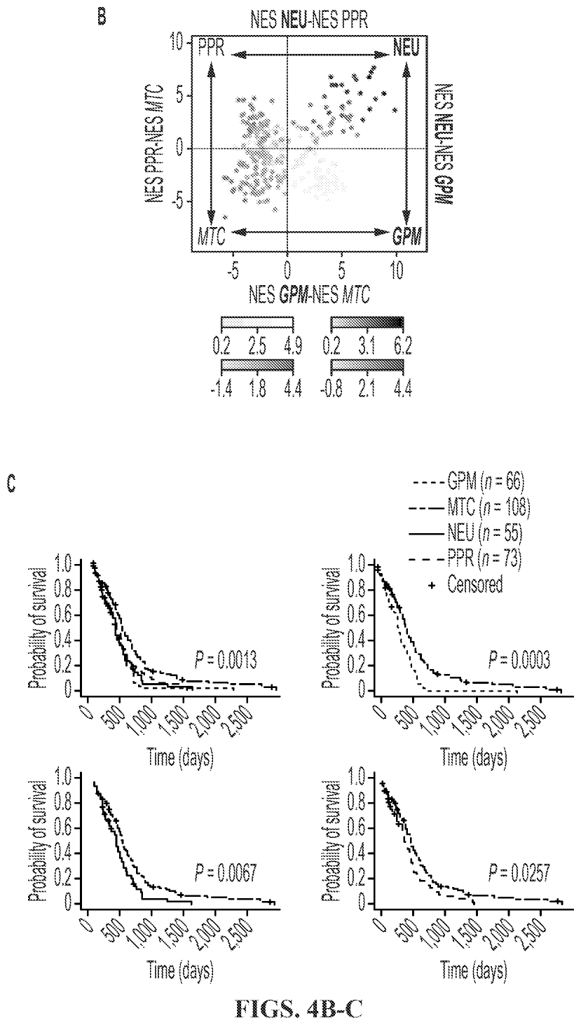

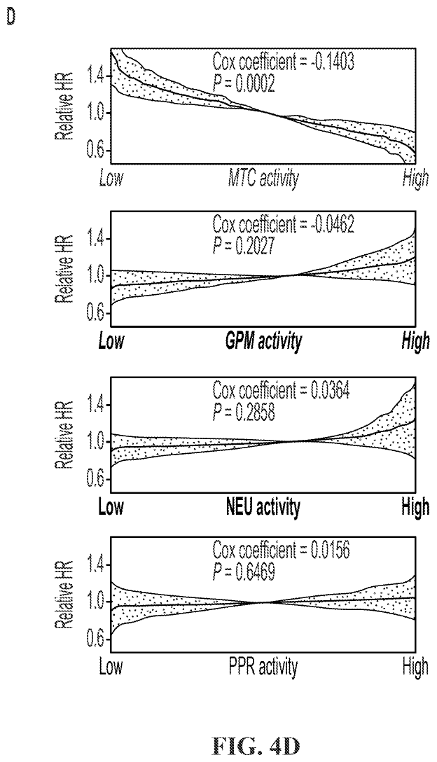

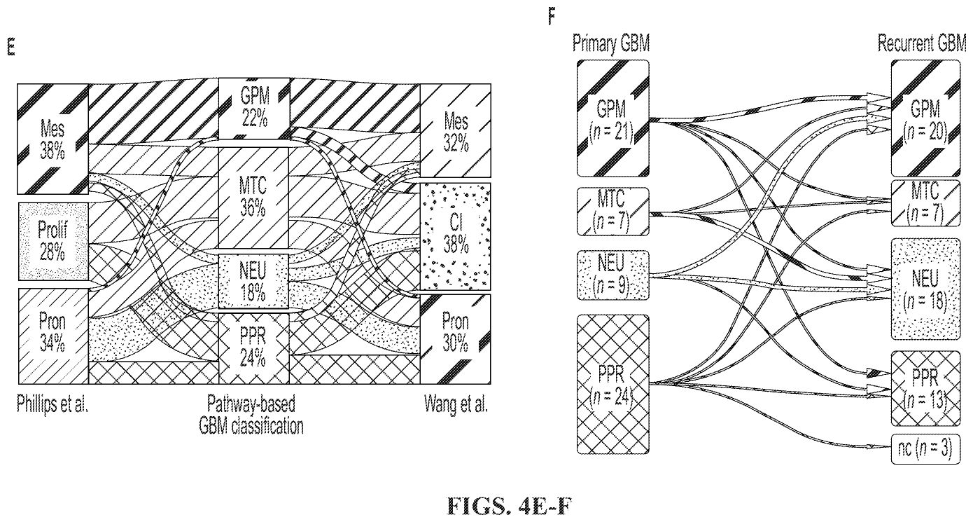

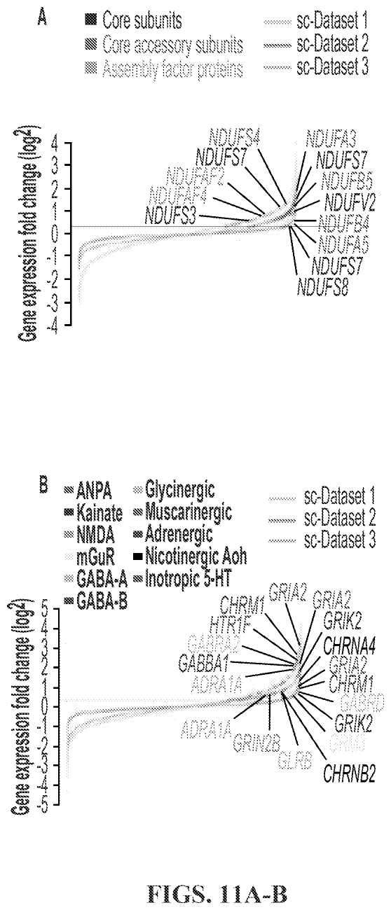

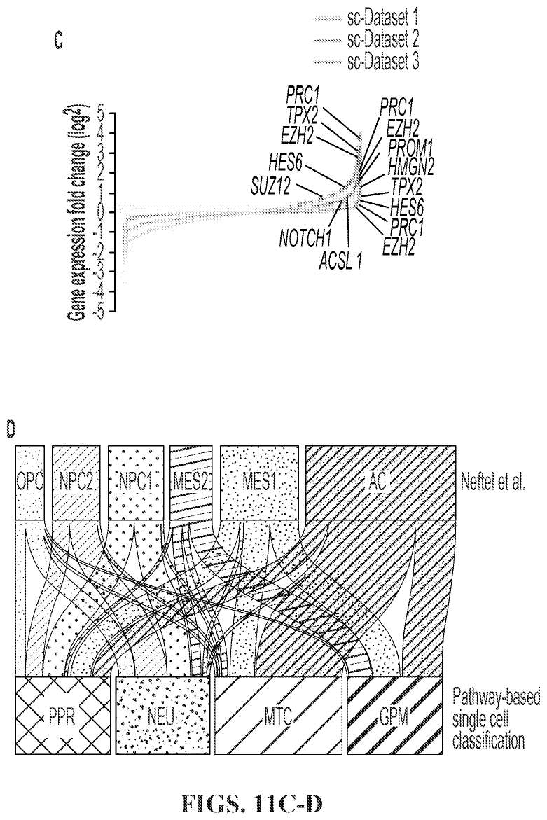

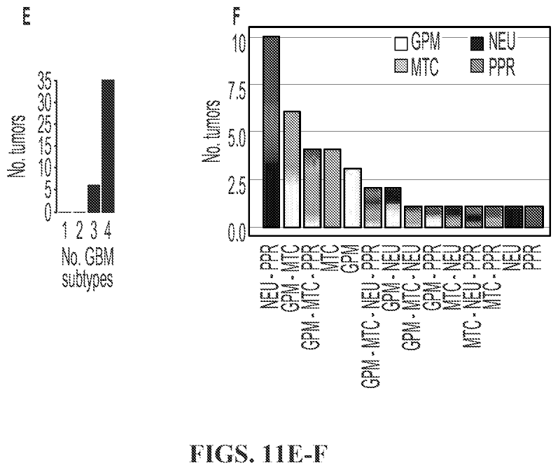

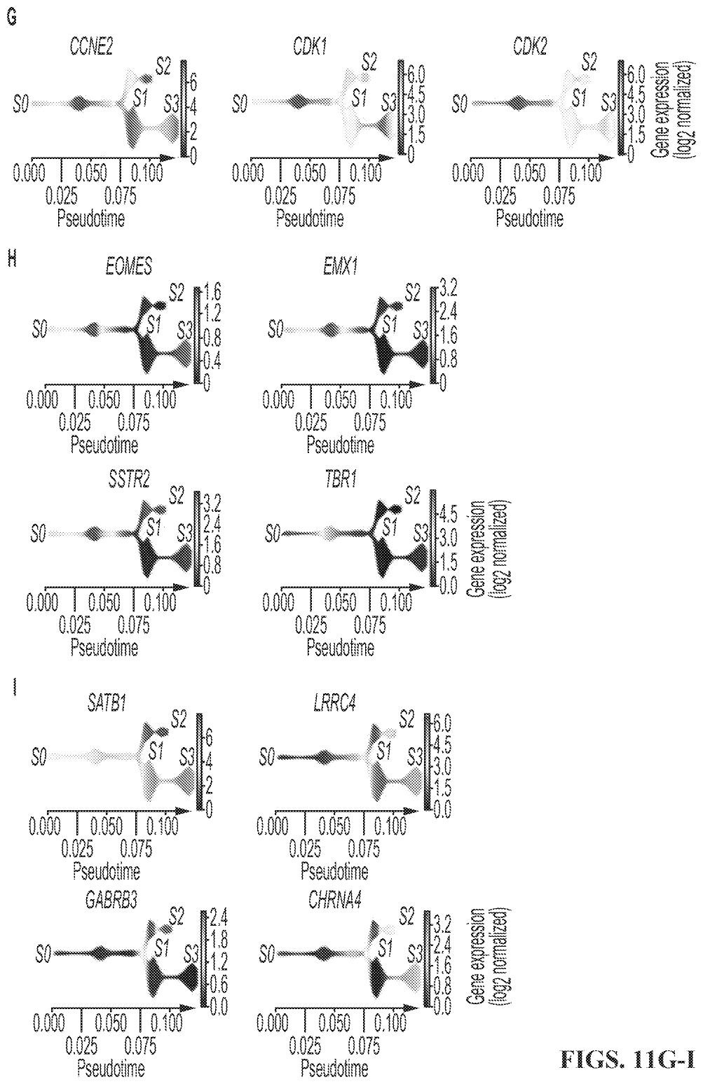

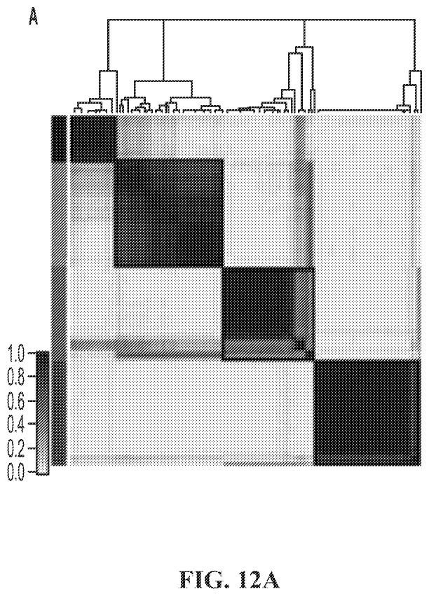

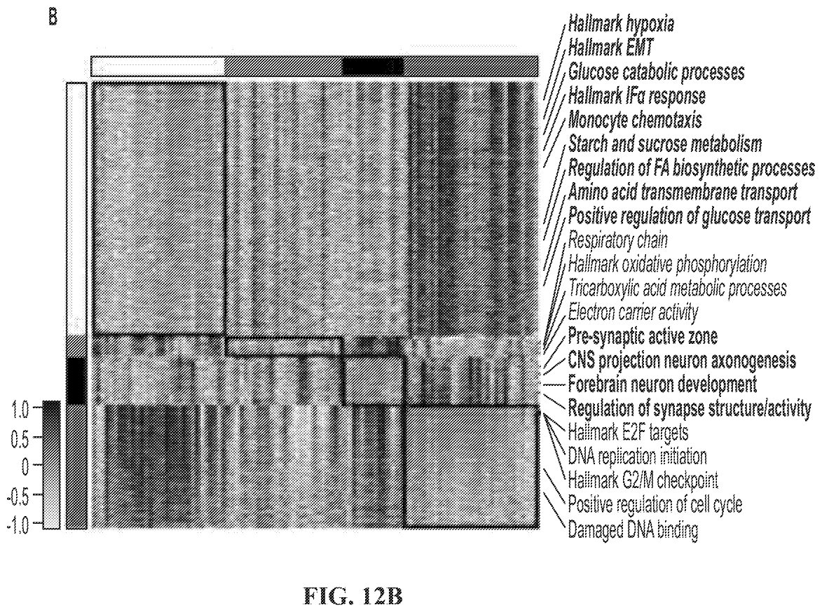

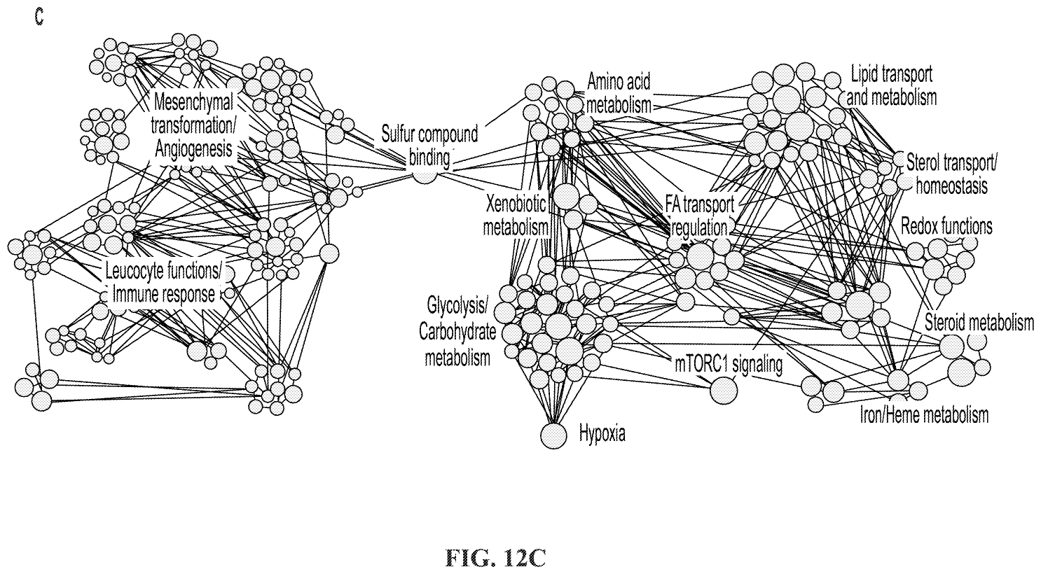

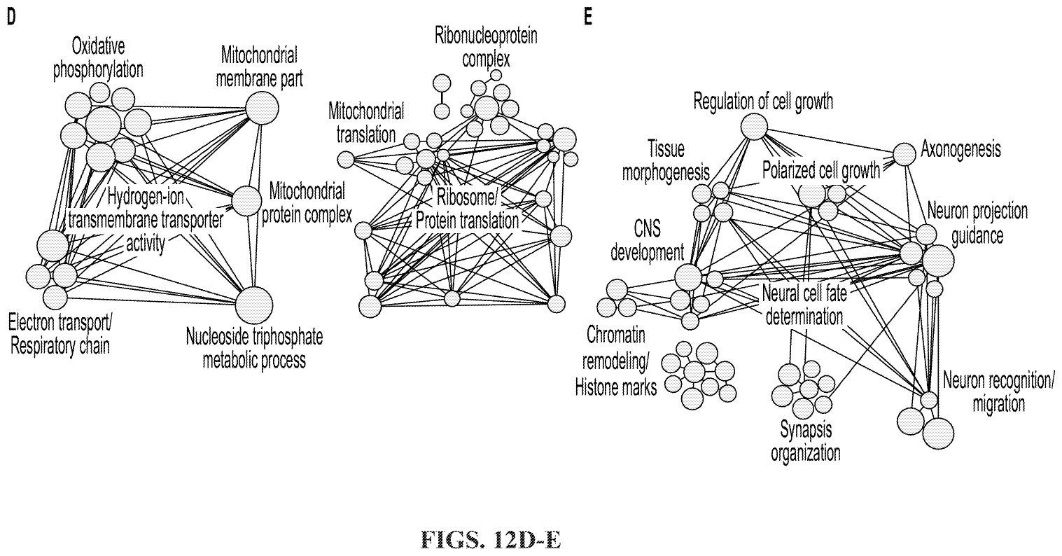

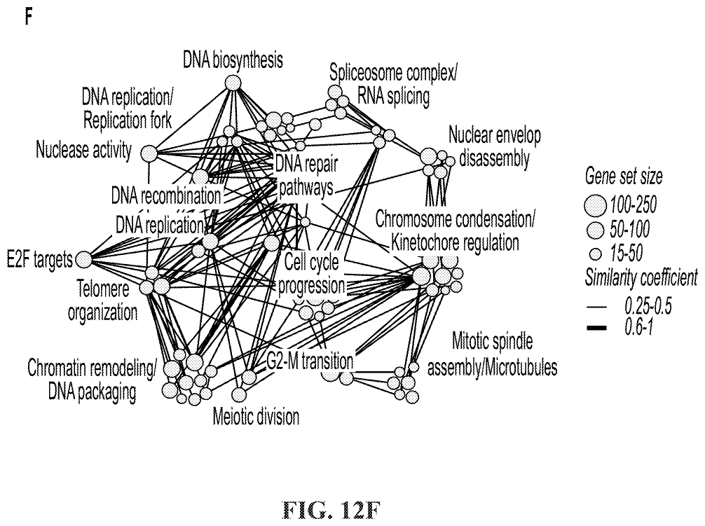

To generate an unbiased classification of GBM that encapsulates cellular states fundamental to glioma biology, we sought to identify key phenotypic patterns from scRNA-seq data including 36 adult high-grade gliomas (17,367 single glioma cells) from three independent datasets^{4,6,7}. To define the core biological state of individual glioma cells, we developed an unbiased computational approach (single-cell biological pathway deconvolution, scBiPaD) that scored the activity of 5,032 pathways in each cell and grouped cells with similar biological pathway enrichment (FIG. 10). We built a consensus clustering of all cells in each tumor and estimated the closeness of individual tumor clusters. We assigned 91% of the cells to four distinct clusters, which we defined as glycolytic/plurimetabolic (GPM, marked in red throughout), mitochondrial (MTC, green), neuronal (NEU, blue) and proliferative/progenitor (PPR, cyan) based on the most active biological functions in each cluster (FIGS. 2A,B). The same four-cluster distribution and biological membership was obtained when the clustering analysis was applied independently to each scRNA-seq dataset (FIG. 4). The GPM cluster was sustained by a large array of metabolic activities that, in addition to glycolysis/hypoxia-related functions, included the metabolism of lipids, amino acids, steroids, iron and sulfur but excluded mitochondrial/OXPHOS activities (FIG. 2C). This cluster was also enriched in mesenchymal and immune-related functions. Mitochondrial metabolism and OXPHOS were the hallmarks of the MTC cluster that also included fatty acid oxidation and general mitochondrial functions (FIG. 2D). Most subunits of mitochondrial complex I that can be inactivated in cancer cells to generate the Warburg effect⁸ were highly expressed in MTC compared to the other clusters (FIG. 11A). The NEU cluster was uniquely characterized by specialized neuronal functions such as axonogenesis and synaptic transmission (FIG. 2E). Multiple neurotransmitter receptors that have recently been associated with the neuronal functions that promote glioma-neuron synapsis and brain tumor aggressiveness⁹ were specifically elevated in the NEU cluster (FIG. 11B). Finally, the PPR cluster was enriched in pathways associated with cell cycle progression, DNA replication, mitosis and DNA damage repair (FIG. 2F) and markers of neural stem/progenitor cells (FIG. 11C). Recently, scRNA-seq has been used to deconvolute the phenotypic states of GBM cells into six lineage-specific cellular identities: astrocyte-like (AC), mesenchymal-like 1 (Mes1), mesenchymal-like 2 (Mes2), neural progenitor cell-like 1 (NPC1), neural progenitor cell-like 2 (NPC2) and oligodendrocyte progenitor cell-like (OPC)⁴. We examined the relationship between pathway-based and lineage-specific cellular states. The neurodevelopmental association of PPR and NEU states was evident from the enrichment in NPC1, NPC2 and OPC signatures. Conversely, the GPM and MTC transcriptional states exhibited preferential enrichment with the IVIES and AC cell state, respectively (FIG. 11D).

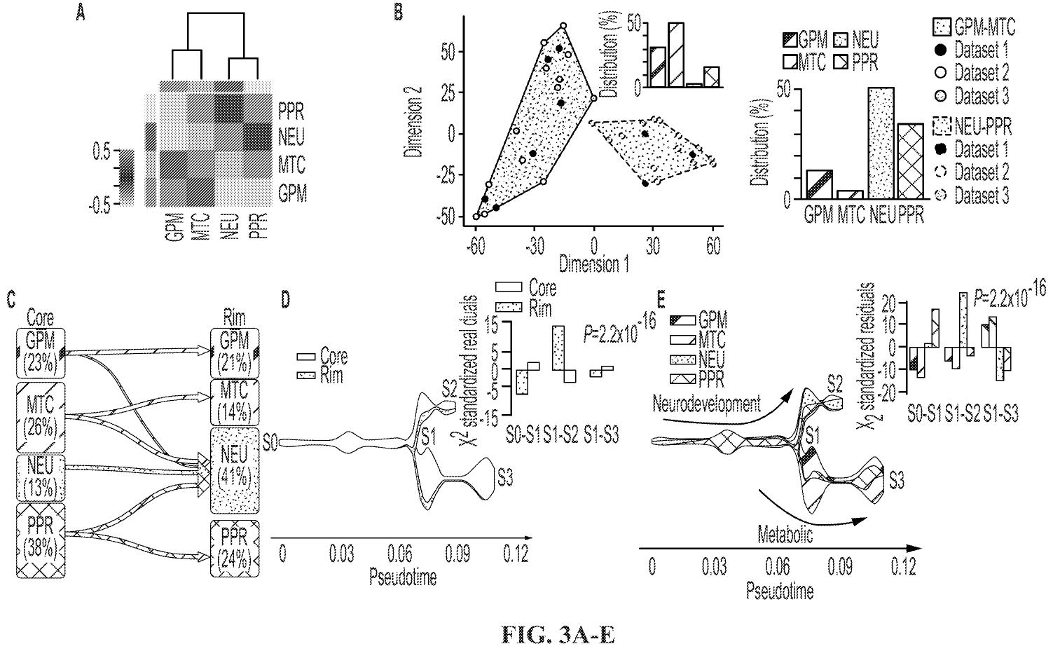

Next, we determined the stability of cell states within each of the 36 tumors by computing the fraction of cells assigned to each state. Although each tumor contained four or three different cell states (FIG. 11E), most expressed a dominant state together with variable, smaller cell fractions corresponding to different states. The estimation of the frequency of coexistence of cell states within individual tumors showed two distinct patterns characterized by the preferential coexistence of GPM with MTC cells and PPR with NEU cells, respectively (FIGS. 3A,B and FIG. 11F).

To determine whether biological branches are spatially segregated within glioma tumors, we analyzed 33 tumor core samples and 14 matched invasive rims obtained by precision navigation surgery from nine patients in dataset 1. The percentage of glioma cells with NEU features increased from 13 to 41 in the peripheral areas of the tumor, whereas PPR and MTC cells decreased (FIG. 3C). By tracing the evolutionary path of the four biological glioma states at tumor core and rim with the STREAM algorithm¹⁰, we obtained two fundamental axes characterized by neurodevelopmental PPR and NEU (S0-S2) and metabolic MTC and GPM states (S1-S3), respectively (FIGS. 3D,E). The neurodevelopmental axis exhibited an evolutionary trajectory defined by a branch enriched in core-derived PPR cells (S0-S1; FIGS. 3D,E) expressing cell cycle genes (CCNE2, CDK1 and CDK2; FIG. 11G) and the transcriptional program of intermediate progenitor cells (EOMES, EMX1 and SSTR2) intermingled with NEU cells expressing markers of newly born neurons (TBR1; FIG. 11H). Conversely, the tract enriched in rim-derived cells (S1-S2; FIGS. 3D,E) consisted of more mature NEU cells expressing markers of specialized neuronal functions (LRRC4/NGL2, SATB1, GABRB3 and CHRNA4; FIG. 11I). The lack of expression of CCNE2 and other cell cycle genes in TBR1-positive cells from core- and rim-enriched mature NEU cells indicates that, regardless of the differentiation stage, NEU are mostly nonproliferating cells (FIGS. 11G-I).

### Pathway-Based Classification of GBM Recapitulates Single-Cell States With the Mitochondrial Subtype Exhibiting Better Survival

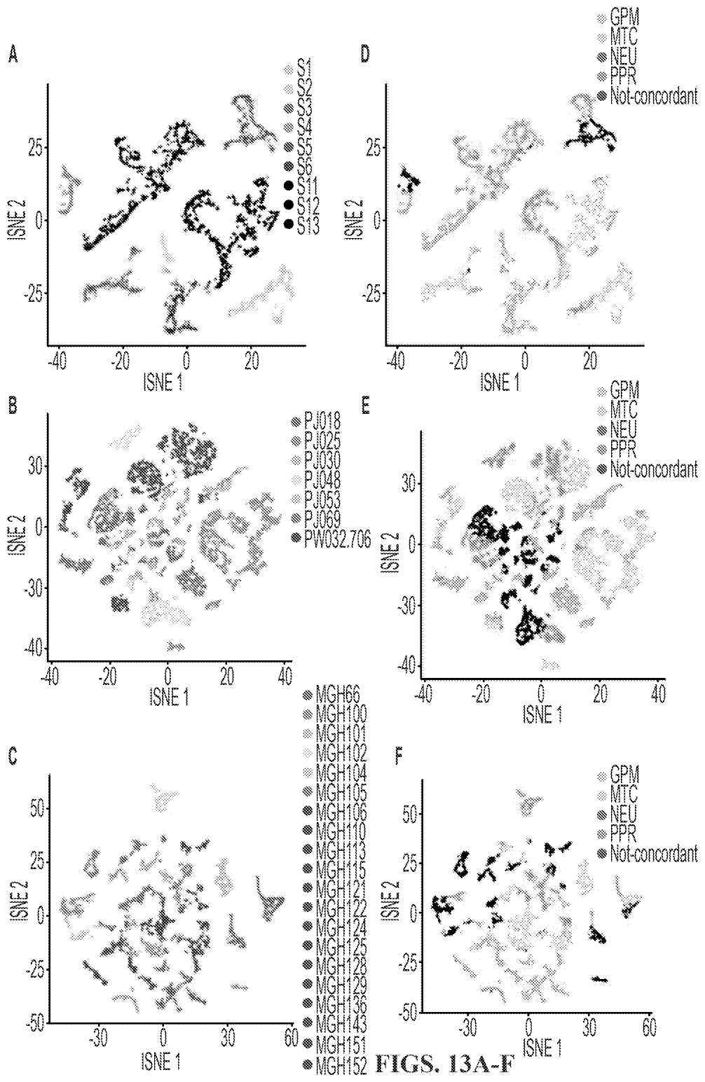

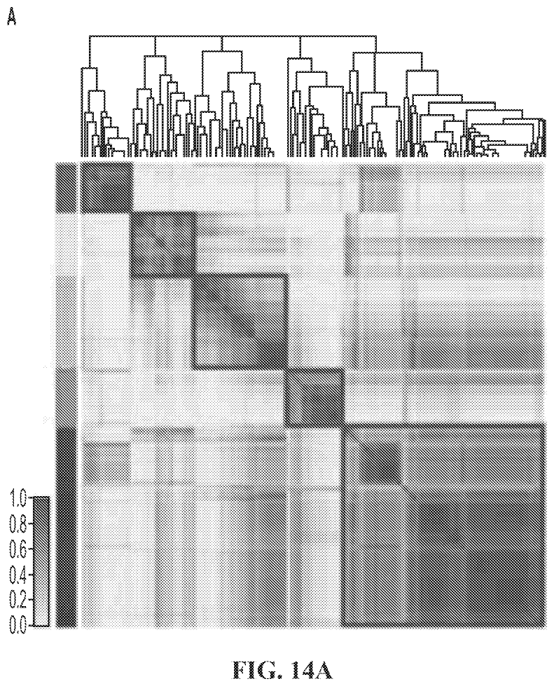

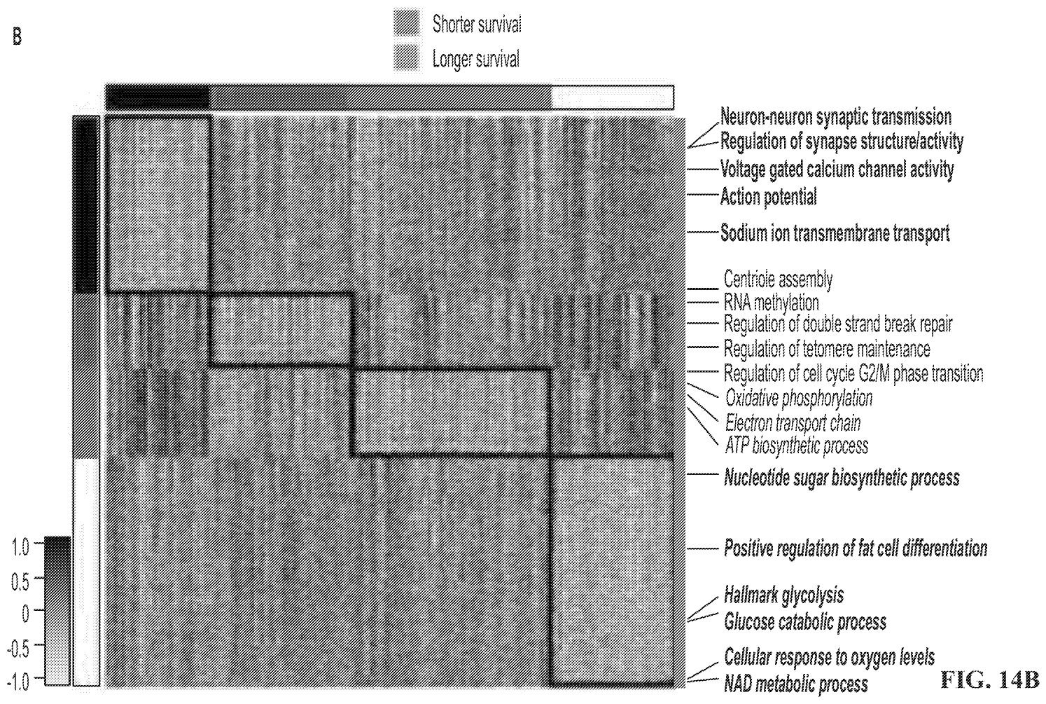

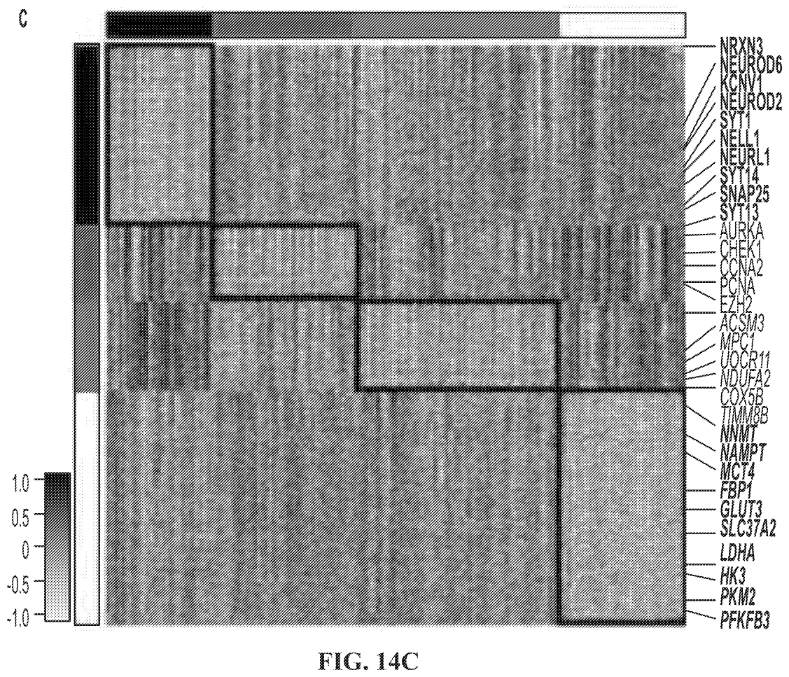

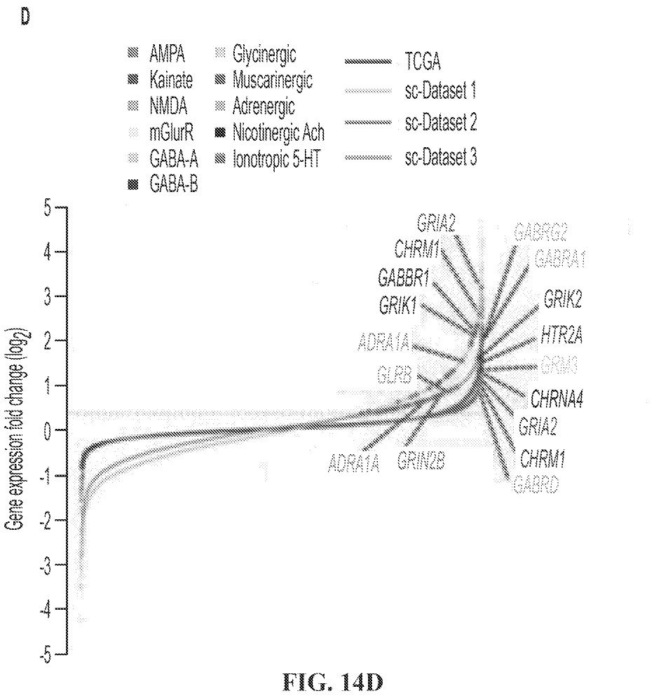

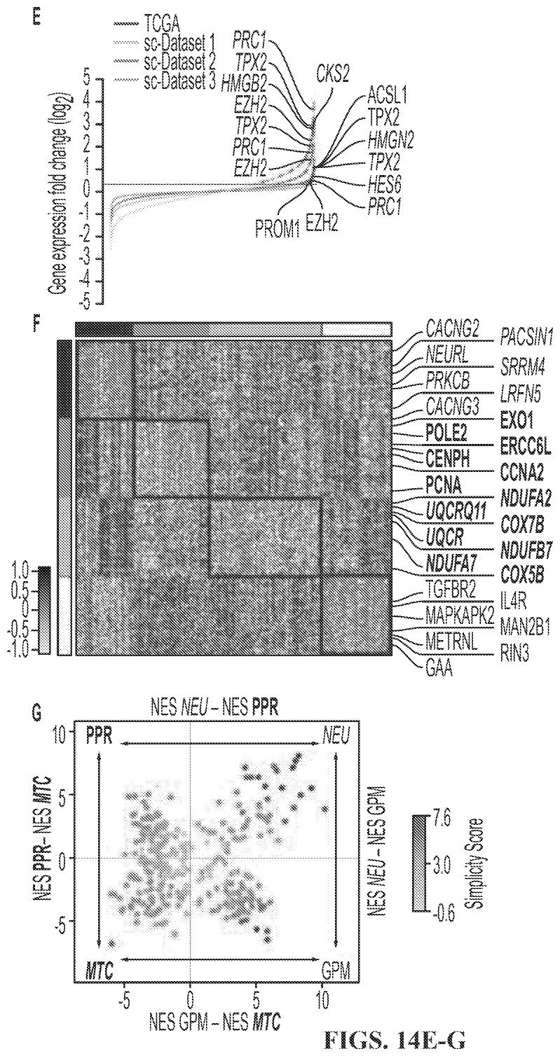

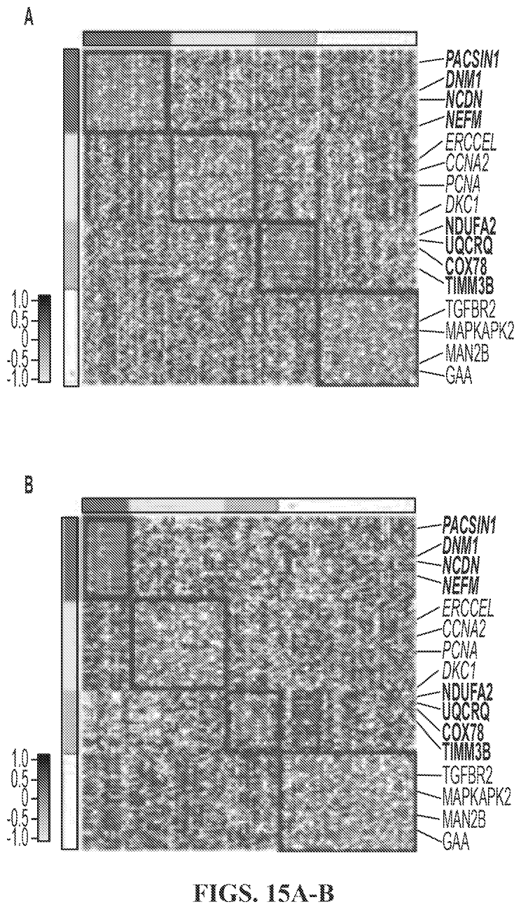

We designed a pathway-level analysis that could cluster primary GBM on the basis of shared pathways associated with clinical outcome. Using 534 Cancer Genome Atlas (TCGA) IDH wild-type GBM and the single-sample Mann-Whitney-Wilcoxon gene set test (ssMWW-GST), we selected 192 of 5,032 pathways whose activity (increased or decreased) was significantly associated with patient survival (P<0.05, log-rank test). By the application of scBiPaD to single-cell datasets (FIGS. 13A-F), we confirmed that this reduced configuration of biological activities captured the dominant cell states with 92% concordance when compared with the analysis of 5,032 pathways (FIGS. 13A-F). Next, we classified 534 primary GBM by building a consensus clustering on pathway enrichment score (FIG. 14A). We obtained four GBM subgroups that included 304 tumors (62% of the cohort) defined by differentially active, survival-associated pathways (FIG. 14B). The biological functions of each of the four sets of pathways recapitulated the activities identified by single-cell analysis, including NEU (blue), PPR (cyan), MTC (green) and GPM (red) (FIG. 4A). Consistently, genes upregulated in each cluster were markers and effectors of the highlighted biological activities, including neurotransmitter receptors and neural stem/progenitor cell markers for NEU and PPR single-cell states, respectively (FIGS. 14C-F). To evaluate the strength of the dominant state in bulk tumors, we computed a ‘simplicity score’ for each sample as a continuous measure of the strength of the state 3 within a four-quadrant plot corresponding to the four transcriptomic states (FIG. 14G). The coherent quadrant/state clustering of tumors, colored according to enrichment of the prevalent subtype, indicated that the analysis captured the dominant biological feature from the bulk GBM transcriptome (FIG. 4B).

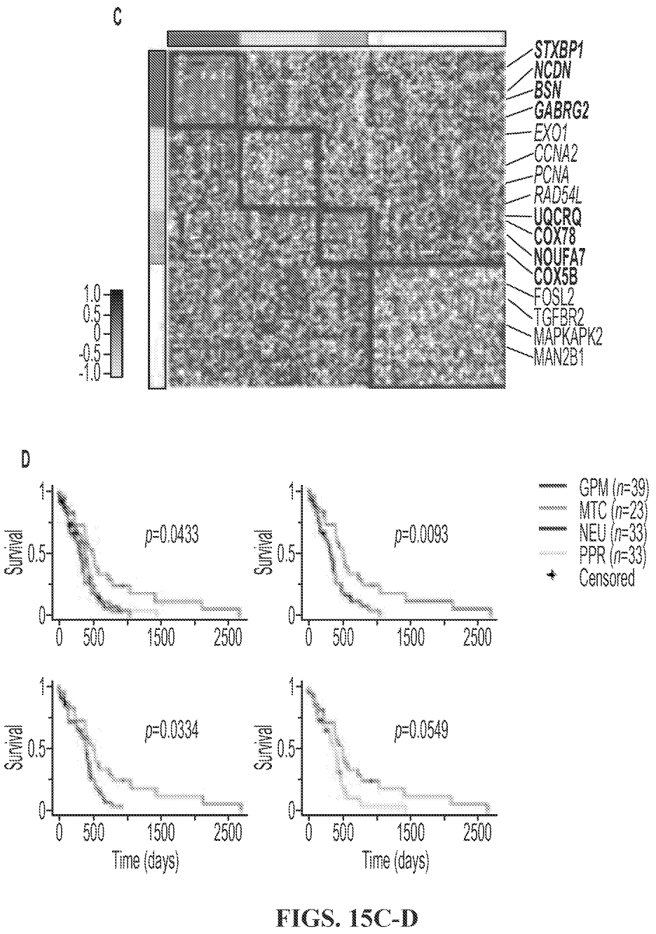

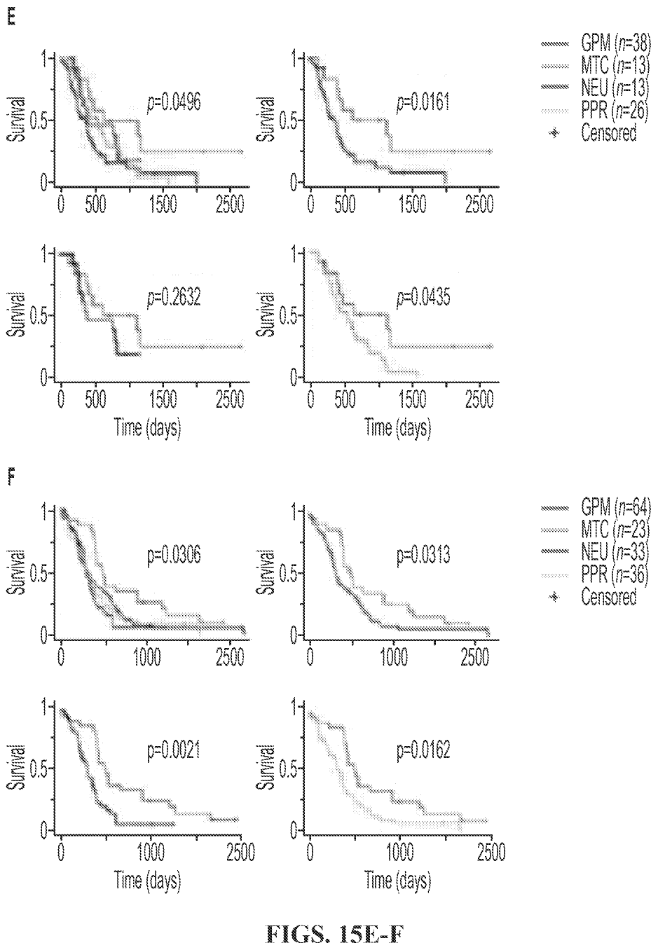

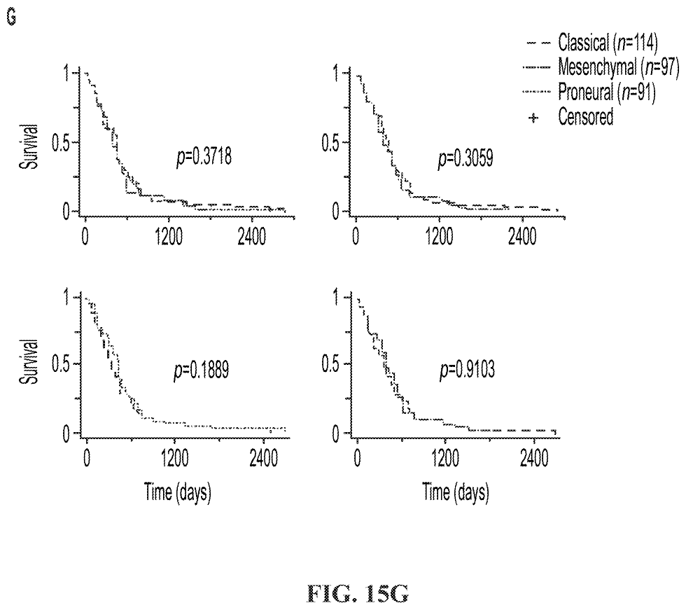

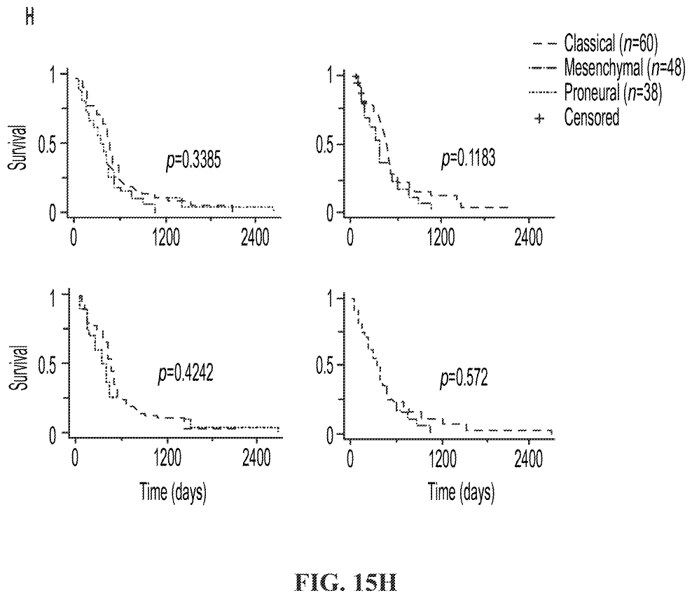

To determine the impact of the pathway-based classification on clinical outcome, we used several parameters. First, Kaplan-Meier estimation and log-rank testing showed significantly better survival for MTC GBM, either when all four subgroups were included in the analysis or in pairwise comparison (FIG. 4C). Second, Cox proportional hazards models including MTC, GPM, NEU and PPR activities as independent continuous covariates showed that the only significant variable that predicted survival was MTC, with increasing activity estimating a decreasing risk of death (hazard ratio (HR)=0.87, Cox coefficient=−0.14, P=0.0002; FIG. 4D). All other transcriptomic activities exhibited a trend for higher risk of death, without reaching statistical significance. Third, in a multivariate analysis, the impact of MTC state on survival was independent of age, gender and methyl guanine methyl transferase (MGMT) Q11 methylation, factors that affect survival of patients with IDH wild-type GBM. We also failed to find significant associations between the transcriptional subtypes and clinical and molecular characteristics of GBM. Using a 50-gene signature obtained from the ranked list of each bulk GBM subtype, we confirmed the four-group classification and the more favorable clinical outcome of MTC GBM in three additional GBM datasets (FIGS. 15A-F).

To assess the intersection of the pathway-based classification with the two existing and widely used GBM classifiers^{3,11}, we compared subclass assignment of 304 GBM from TCGA using each of the three classifiers. The two previously established classifications recognized three groups named after signature genes, including mesenchymal and proneural subtypes, but they differ in the attribution of the third group as proliferative in the classification of Phillips et al.¹¹, and classical in the classifier by Wang et al.³. The MTC subtype uncovered by the pathway-based classification was orthogonally distributed across the three subgroups, suggesting that OXPHOS programs are not restricted to a specific cell identity (FIG. 4E). We also found a positive association between the mesenchymal subgroups of both classifications and the GPM subgroup, suggesting that mesenchymal identity and GPM activity are inseparable features in GBM. Proneural and proliferative, and proneural and classical, subtypes of the two classifications incorporated similar fractions of NEU and PPR, respectively. NEU and PPR were mostly excluded from the mesenchymal groups. In contrast to the pathway-based classifier, neither classification captured differences in survival in the TCGA IDH wild-type GBM cohorts (FIGE. 15G-J).

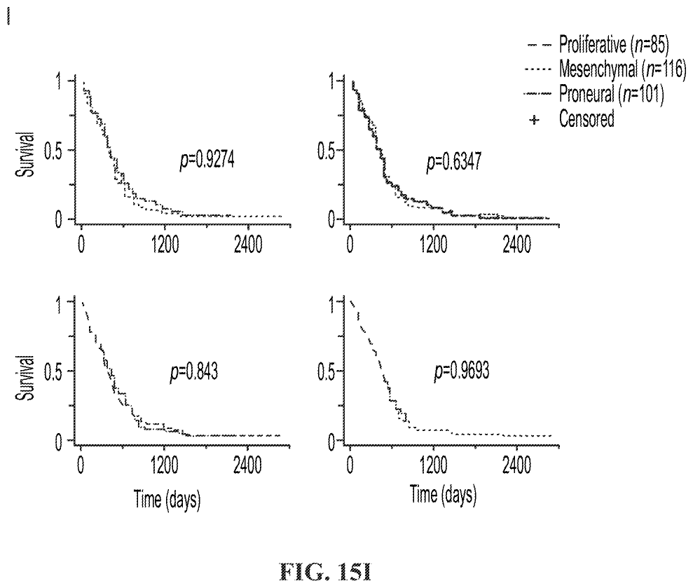

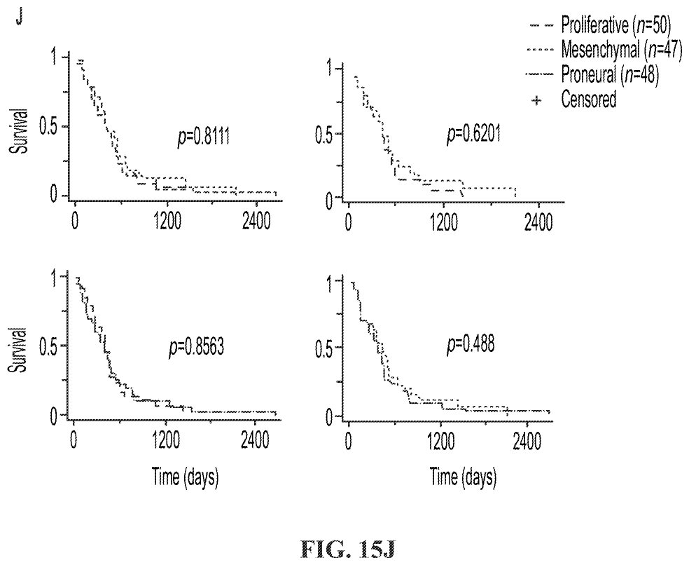

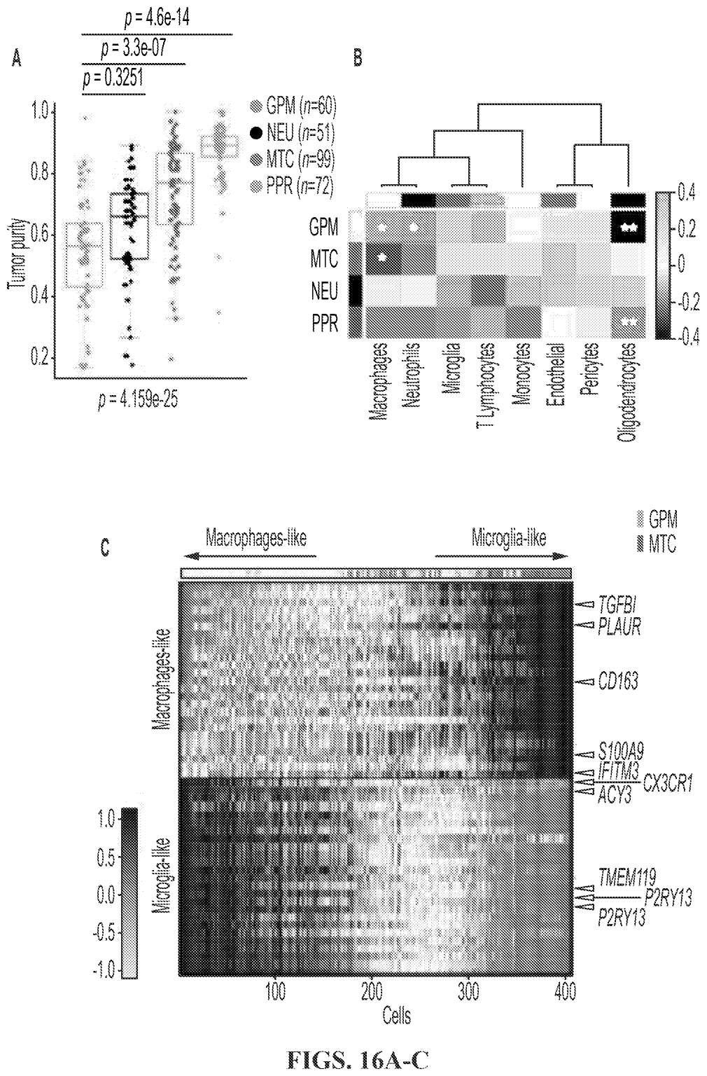

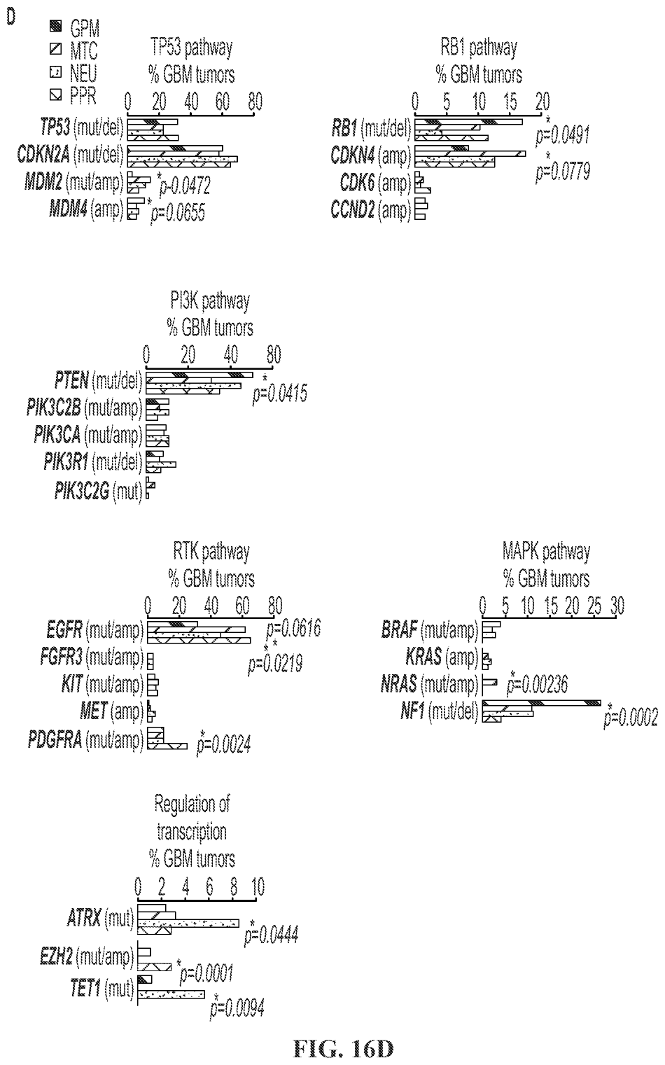

To determine whether the transcriptomic state of tumor cells affects the extent and/or composition of the tumor microenvironment (TME), we inferred the fraction of stromal/immune cells and consequently tumor cell purity by applying AB SOLUTE¹². GPM GBM had the lowest tumor purity followed, in increasing order, by NEU, MTC and PPR (FIG. 16A). Next, we used scRNA-seq data to characterize the cellular components of the TME in each GBM subtype¹³⁻¹⁵. GPM was marginally associated with macrophage and neutrophil infiltration, while the PPR subtype was associated with the presence of oligodendrocytes (FIG. 16B). To determine whether tumors exhibiting high glycolytic or OXPHOS activity differ in the extent of infiltration of macrophages and microglia, the predominant nonmalignant cells in the glioma TME^{7,16}, we scored myeloid cells for the relative expression of macrophage- and microglia-specific genes in four samples from dataset 1, two containing >75% GPM tumor cells (S4_D1 and S12_D1) and two predominantly composed of MTC tumor cells (S1_D1 and S5_D1; FIG. 16C). Consistent with previous findings^{7,16}, the two cell types presented a continuum distribution. However, macrophage-like cells were mainly restricted to the GPM TME whereas the less abundant MTC TME was enriched in microglia-like cells.

Finally, to rationalize the biological variations imposed by tumor progression, we examined 61 matched primary and recurrent GBM17. The evolutionary trajectory of recurrent GBM was marked by a reduction in PPR (from 39 to 21%) and gain of NEU states (from 15 to 29.5%; FIG. 4F). Moreover, recurrent NEU GBMs had a significantly higher neuronal NES than primary NEU (3.59±1.74 and 1.80±1.15 in recurrent and primary tumors, respectively; P=0.005, two-sided MWW test) and a stronger enrichment in synaptic activities (1.60±0.39 and 1.42±0.36 in recurrent and primary tumors, respectively; P=0.00005, two-sided MWW test), implicating glioma cells with features of advanced neuronal differentiation in tumor progression.

### Biologically Coordinated Multiomics Features are Associated with GBM Subgroups

To establish the genomic alterations that drive each GBM subtype, we selected copy number variations (CNVs) that impact gene expression in cis (functional CNV, fCNV) and somatic pathogenic single-nucleotide variations (SNVs)¹⁸ and integrated fCNVs and SNVs. The analysis of association between genetic alterations of established GBM driver genes and the four subtypes showed that the GPM subgroup was enriched in deletions and mutations of PTEN, RB1 and NF1, and amplification of MDM4 (FIG. 16D). Mutations of NRAS were exclusive of the MTC subgroup, and amplifications of CDK4/MDM2 were more frequent in this subgroup. ATRX and TETI mutations were associated with the NEU subtype. Finally, the PPR subtype was associated with amplifications and mutations of PDGFRA and EZH2. Amplification and mutations of EGFR were more frequent in subtypes MTC and PPR.

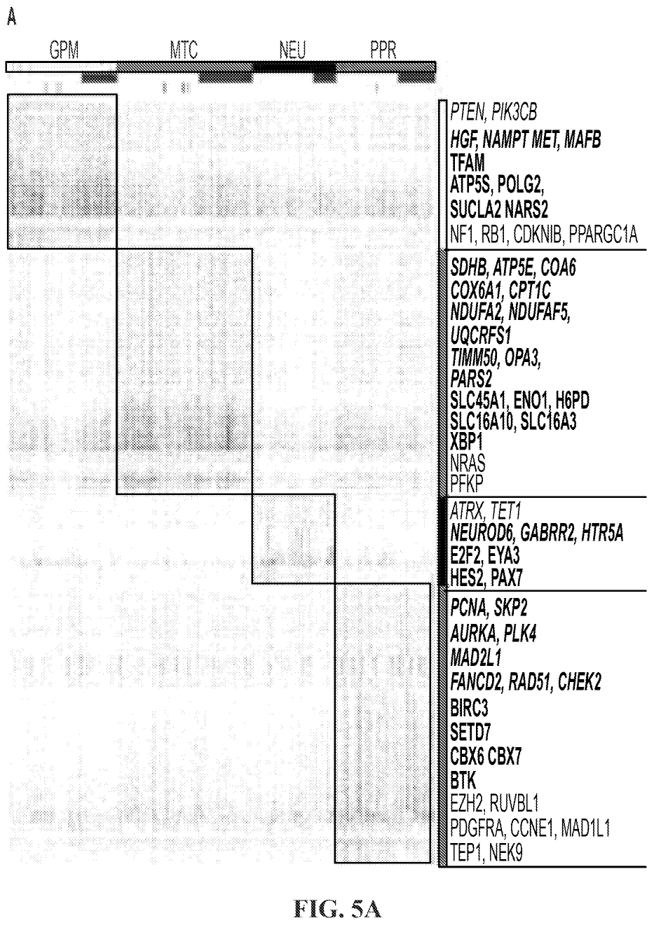

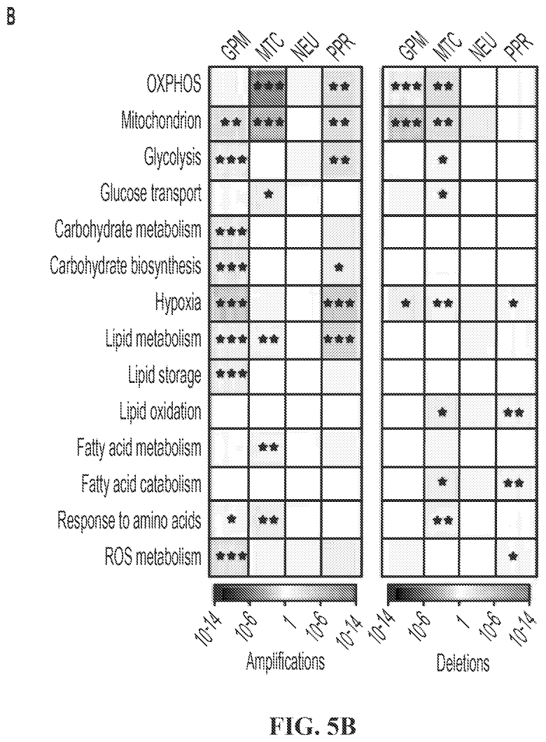

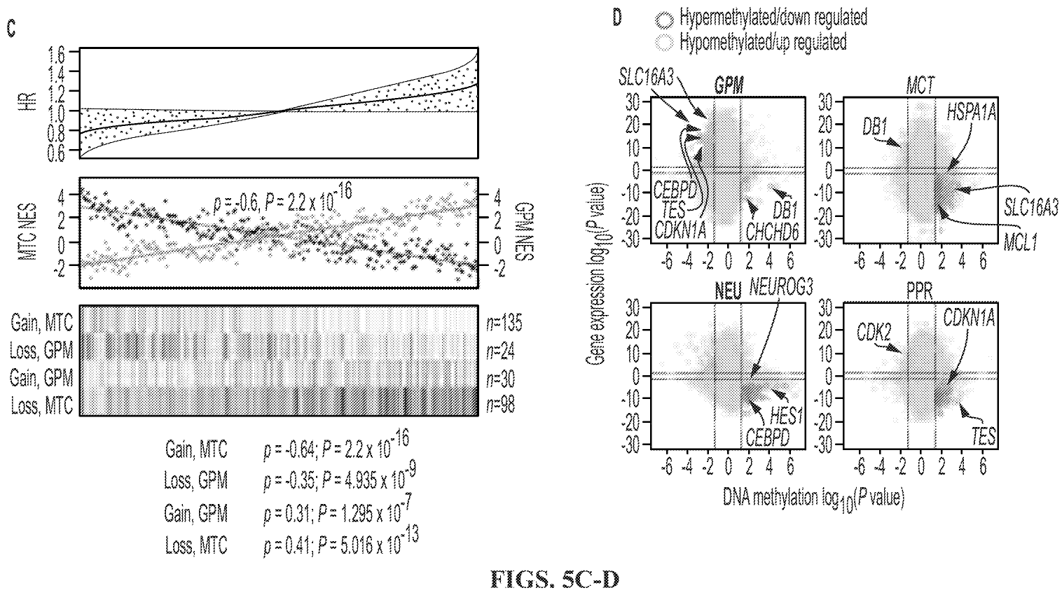

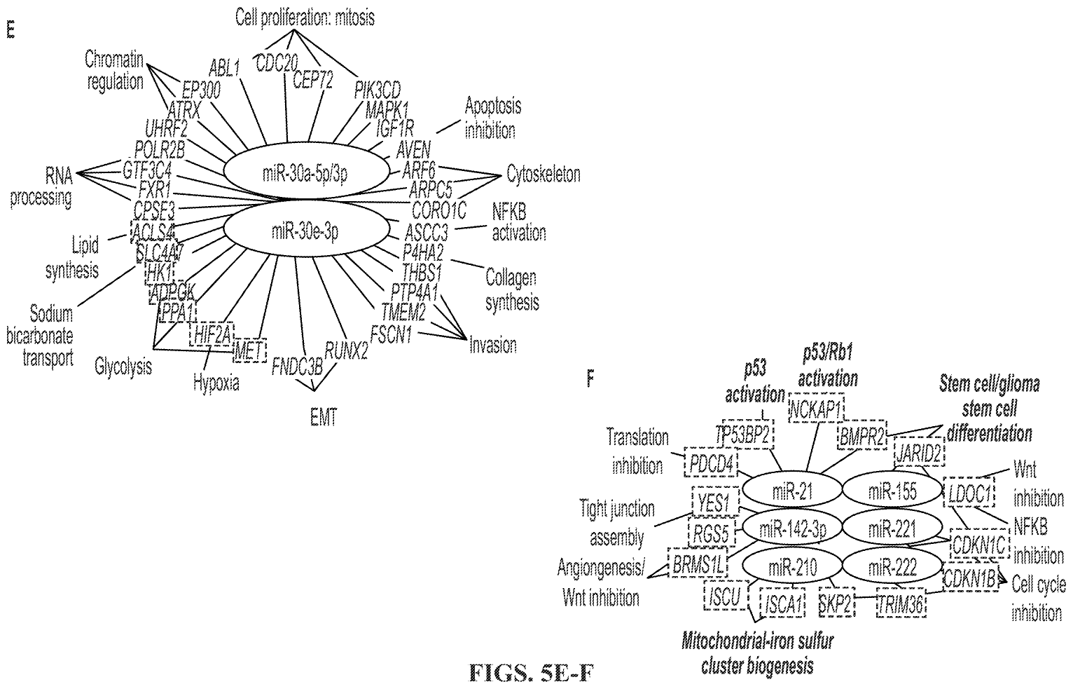

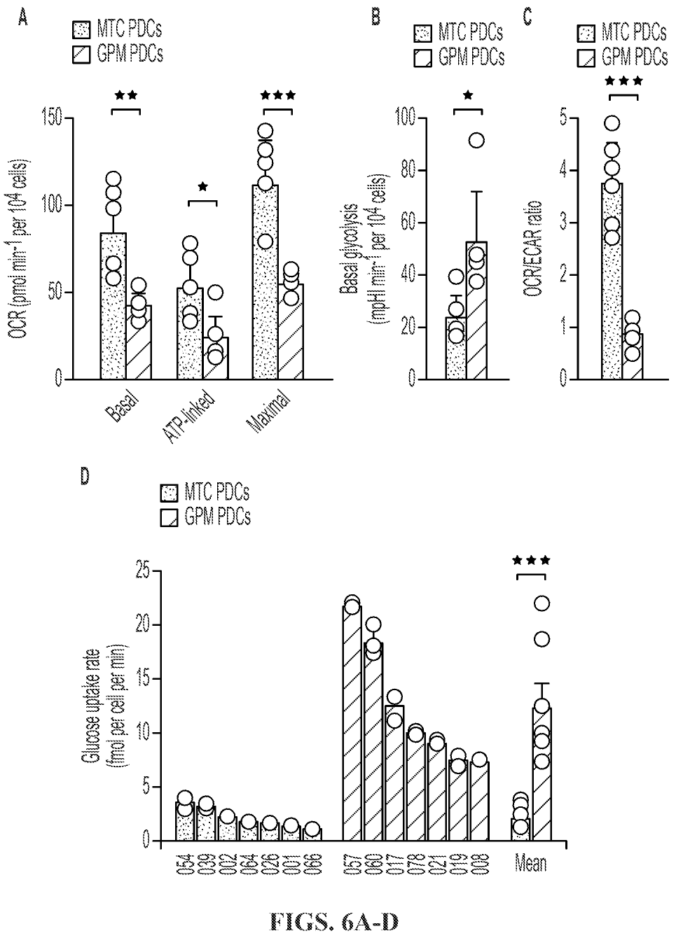

Beside GBM drivers, each subtype harbored a specific repertoire of fCNVs and SNVs, largely composed of alterations of biologically coherent genes (FIG. 5A). Besides fCN gain of the stem/progenitor cell drivers PDGFRA and EZH2, the PPR group was enriched in amplification of activators of cell cycle and mitotic progression (PCNA, SKP2, AURKA and PLK4). Conversely, the NEU subtype was enriched in fCN gain of genes involved in either neuronal cell fate (NEUROD6) or coding for neurotransmitter receptors (GABRR2 and HTR5A). It also harbored/UN loss of genes that normally function in the prevention of neuronal differentiation (HES2 and PAX7). GPM and MTC subgroups exhibited enrichment in biologically antagonistic genetic alterations (FIGS. 5A,B). Thus, GPM GBM harbored/UN gain of genes implicated in glycolysis and carbohydrate metabolism, lipid storage and metabolism and amino acid and reactive oxygen species (ROS) metabolism, and of genes in the hypoxia response pathway, while genes associated with similar metabolic activities were selected as fCN loss in the MTC subtype. In contrast fCN gain in MTC GBM was enriched in OXPHOS and mitochondrial functions, but genes in these categories harbored/UN loss in GPM GBM (FIG. 5B). Some of the genes harboring recurrent and divergent genetic alterations in the GPM and MTC subgroups are candidate drivers of the respective metabolic phenotypes. Notable examples include NAMPT and HGF (JCN gain), TFAM (fCN loss) and PPARGCIA (fCN loss and mutation) in GPM GBM; and SDHB, NDUFA2, NDUFA5, UQCRFSI (fCN gain), ENO, H6PD, SLC 16A3IMCT4, XBP I (fCN loss) and PFKP (fCN loss and mutation) in mitochondrial GBM (FIG. 5A). The contrasting biology of the GPM and MTC states emerged also from a focused analysis of MTC and GPM subgroups, showing that maximal activity of MTC signature was associated with mimimal GPM signature activity (Spearman correlation p=−0.6, P=2.2×10^{-16;} FIG. 5C), reciprocal fCN gain of mitochondrial and fCN loss of glycolytic genes (FIG. 5C) and better clinical outcome (FIG. 5C). Conversely, the maximal activity of genetic GPM signature was associated with mimimal MTC signature activity, reciprocal fCN gain of GPM and fCN loss of MTC genes and shortest survival (FIG. 5C).

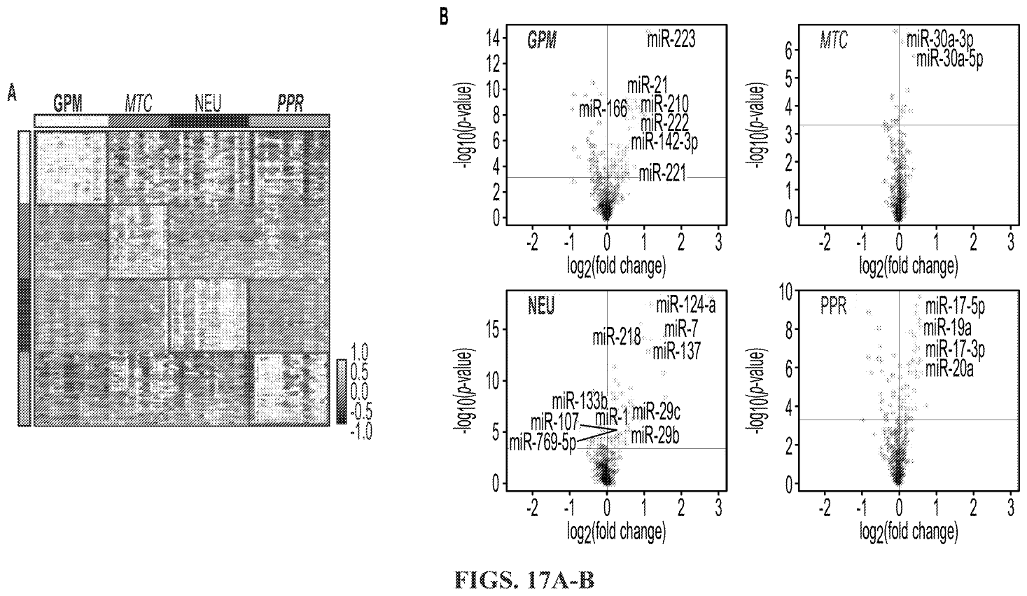

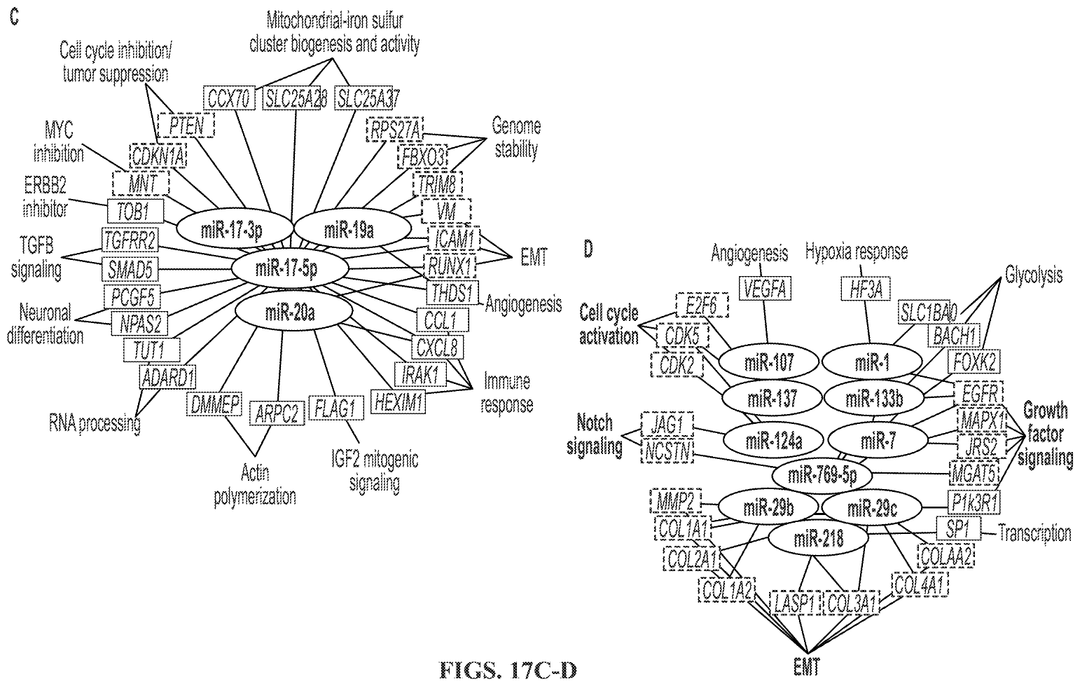

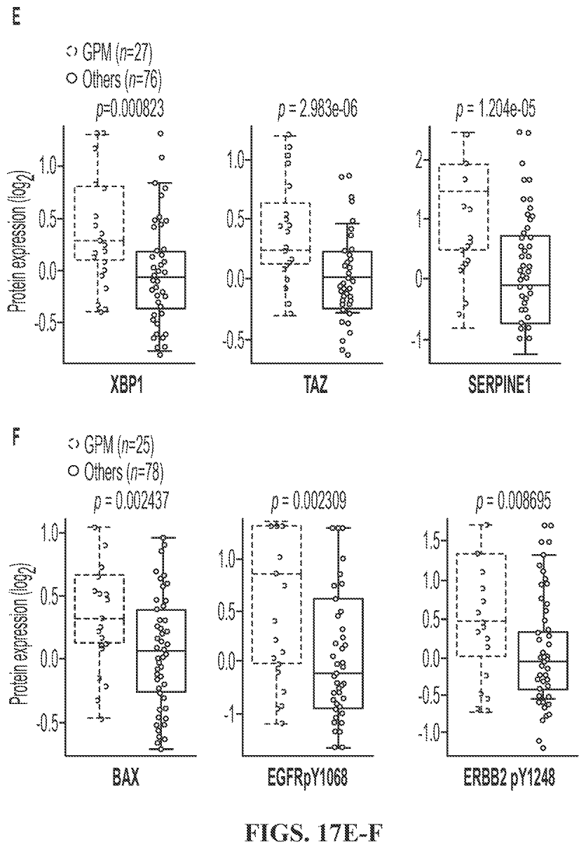

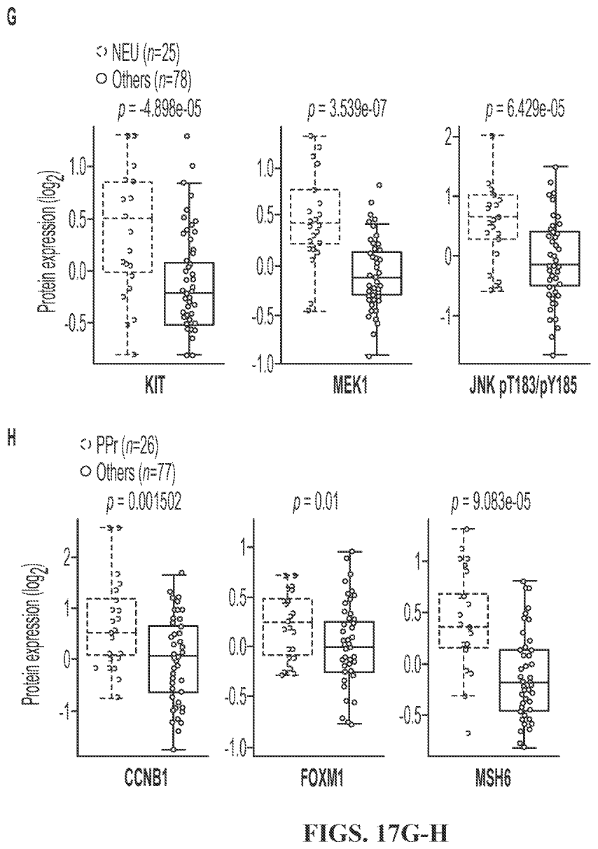

To determine the impact of DNA methylation, we analyzed TCGA GBM samples profiled with the 450 k DNA methylation array. The four GBM subtypes were associated with distinct DNA methylation clusters exhibiting differential methylation of regulatory promoter sequences (FIG. 17A) and subgroup-specific DNA hypermethylation and transcriptional repression in promoters of genes linked to the functional activity of the groups (FIG. 5D).

We also identified subgroup-specific miRNAs (FIG. 17B) and functional miRNA targets (Spearman, ρ<0 and P<0.05). MTC GBM exhibited activation of the miR-30 family of miRNAs (miR-30a-5p/3p and miR-30e-3p), which inhibit glycolysis, the Warburg effect and lipogenesis and promote mitochondrial respiration (FIG. 5E)¹⁹⁻²¹. Conversely, the GPM subtype overexpressed miR-210 and miR-21 and downregulated their target genes (FIG. 5F), promoting stress adaptation and suppression of mitochondrial respiration^{22,23} and inhibiting p53 and mitochondrial apoptosis tumor suppressor pathways', respectively. miR-17-3p and miR-17-5p emerged as regulators of the PPR subtype supporting stemness and cell proliferation by suppression of PTEN and p21 (ref. 25), whereas miR-137, a brain-enriched miRNA with critical functions in neural development and differentiation²⁶, was activated in the NEU subtype (FIGS. 17C,D).

Finally, we explored the reverse-phase protein array (RPPA) platform of TCGA GBM. The transcription factor proteins XBP1 (ref. 27) and TAZ28, which enhance glycolysis and metabolism of glutamine and lipids, were highly expressed in GPM GBM (FIG. 17E); BAX29, master regulator (MR) of mitochondrial-mediated apoptosis, accumulated in MTC (FIG. 17F); KIT30, a receptor with essential functions in neurogenesis, in NEU (FIG. 17G); cyclin B1 (ref. 31) (CCNB1), the key cyclin regulator of mitosis, and FOXM1 (ref 32), driver of stemness of neural and glioma stem cells, in the PPR subtype (FIG. 17H).

### Metabolic GBM Subtypes Have Divergent Mitochondrial, Glucose, Glutamine and Lipid Metabolism

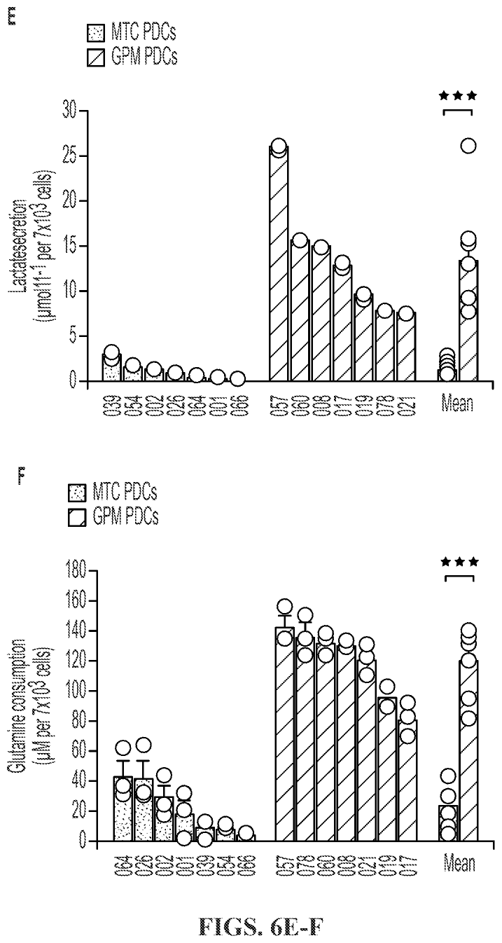

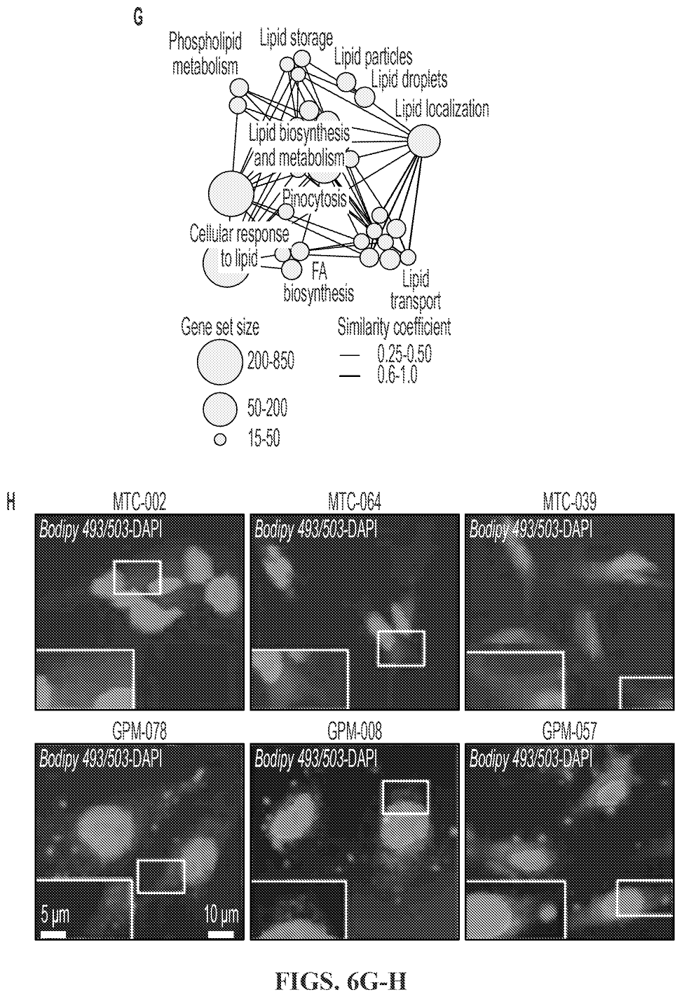

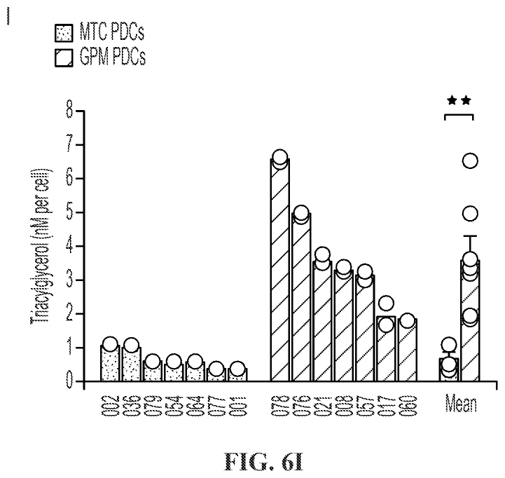

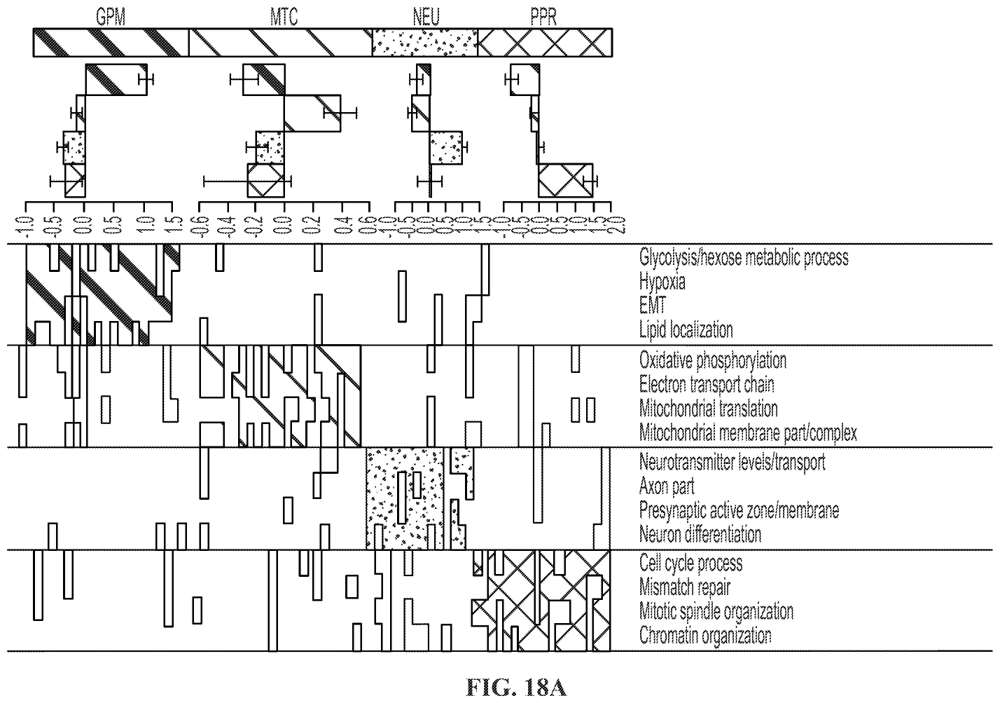

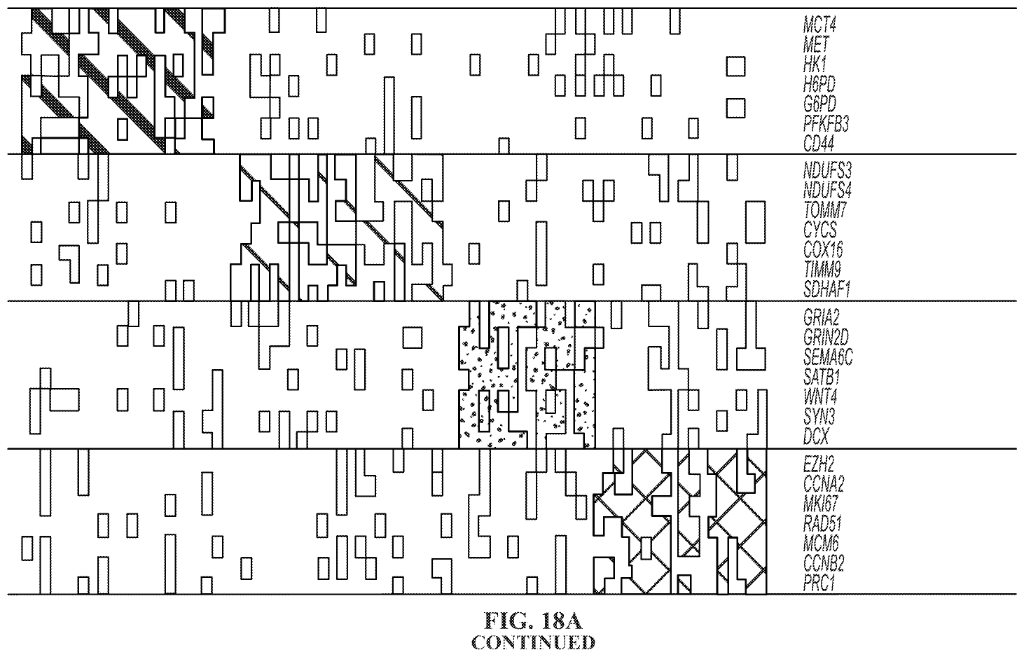

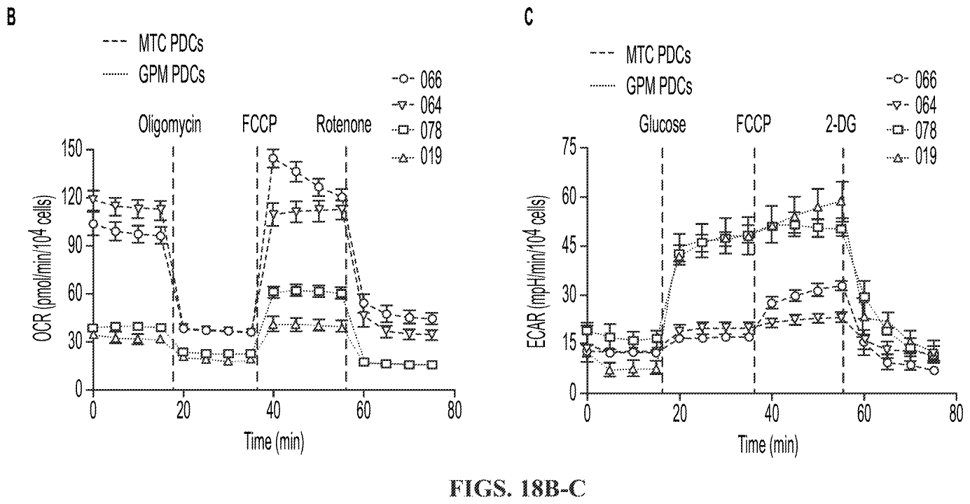

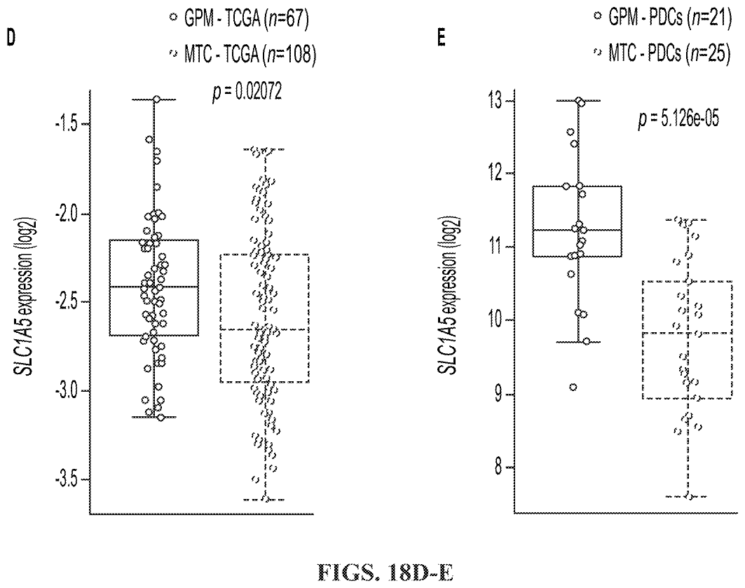

We asked whether the classification of single cells and primary GBM could also be applied to a cohort of patient-derived cellular (PDC) models of GBM. By using a random forest machine learning classifier³³, PDCs partitioned into four groups, each exhibiting enrichment of the corresponding GBM subgroup signature (FIG. 18A), coherent pathway activation (FIG. 18A) and expression of specific marker genes (FIG. 18A). We used MTC and GPM GBM PDCs to experimentally test the metabolic state of these cells using multiple metabolic metrics. MTC PDCs exhibited higher basal, ATP-linked and maximal oxygen consumption rate (OCR) compared with GPM PDCs (FIG. 6A and FIG. 18B). Conversely, basal glycolysis, as indicated by the extracellular acidification rate (ECAR) after glucose addition, was 2.5-fold higher in GPM than in MTC PDCs (FIG. 6B and FIG. 18C). Consequently, MTC PDCs had an OCR/ECAR ratio >3.5-fold higher compared to GPM PDCs (FIG. 6C). GPM PDCs had a sixfold higher rate of glucose uptake and tenfold higher production of lactate than MTC (P=0.0028 for glucose uptake, P=0.0020 for lactate production; FIGS. 6D,E). Glutamine is one of the most important nutrients utilized by cancer cells in the supply of carbon and reduction in nitrogen for biosynthetic reactions and redox homeostasis³⁴, and expression of the major glutamine membrane transporter SLC1A5 was higher in primary GBM and PDC GPM than in MTC (FIGS. 18D,E). Accordingly, estimation of glutamine consumption was higher in GPM than MTC PDCs (5.3-fold, P=0.000002; FIG. 6F).

Among the most prominent set of interconnected metabolic pathways activated in GPM GBM cells and tumors were lipid metabolic activities, especially lipid synthesis and storage (FIG. 6G). In cancer cells, lipid synthesis and storage in lipid droplets that primarily contain triacylglycerides promote survival and growth under adverse conditions³⁵. Thus, we visualized lipid droplets in GPM and MTC PDCs using the lipophilic fluorescent dye BODIPY36 (FIG. 6H) and measured triacylglycerides using a bioluminescent assay (FIG. 61). Both assays detected much higher triacylglyceride content in GPM than in MTC cells (5.4-fold difference, P=0.0032; FIG. 61).

### SLC45A1 Glucose-Proton Symporter on Chromosome 1p36.23 Has Tumor Suppressor Activity in MTC GBM

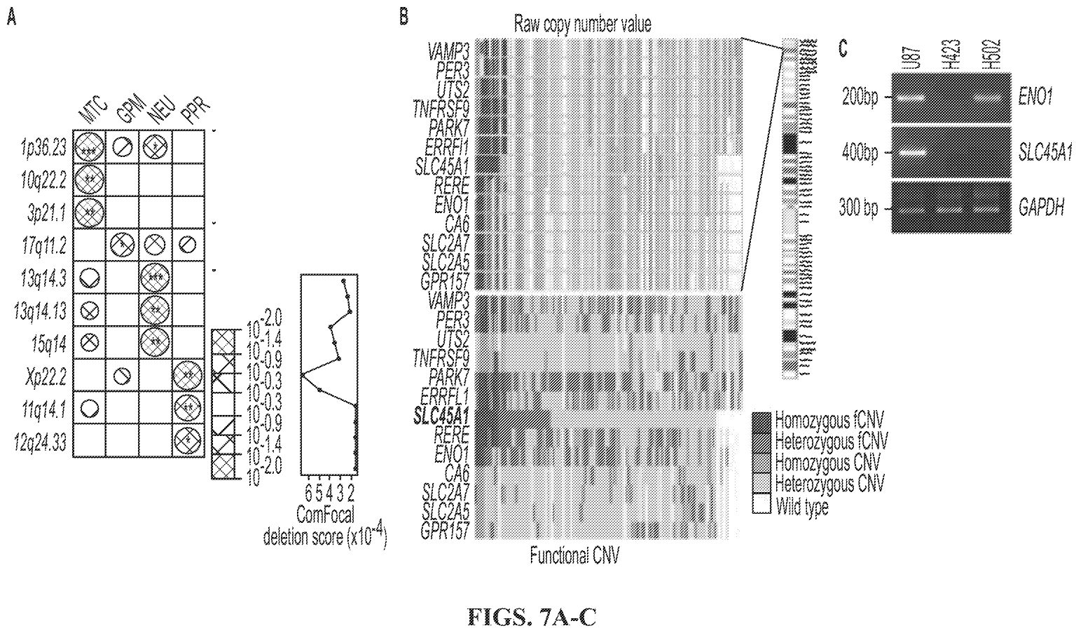

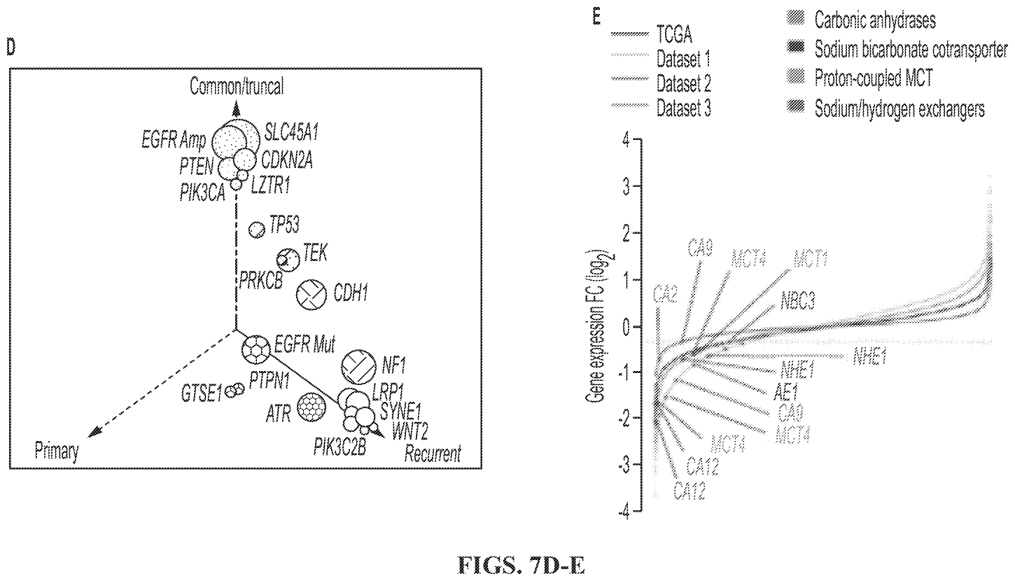

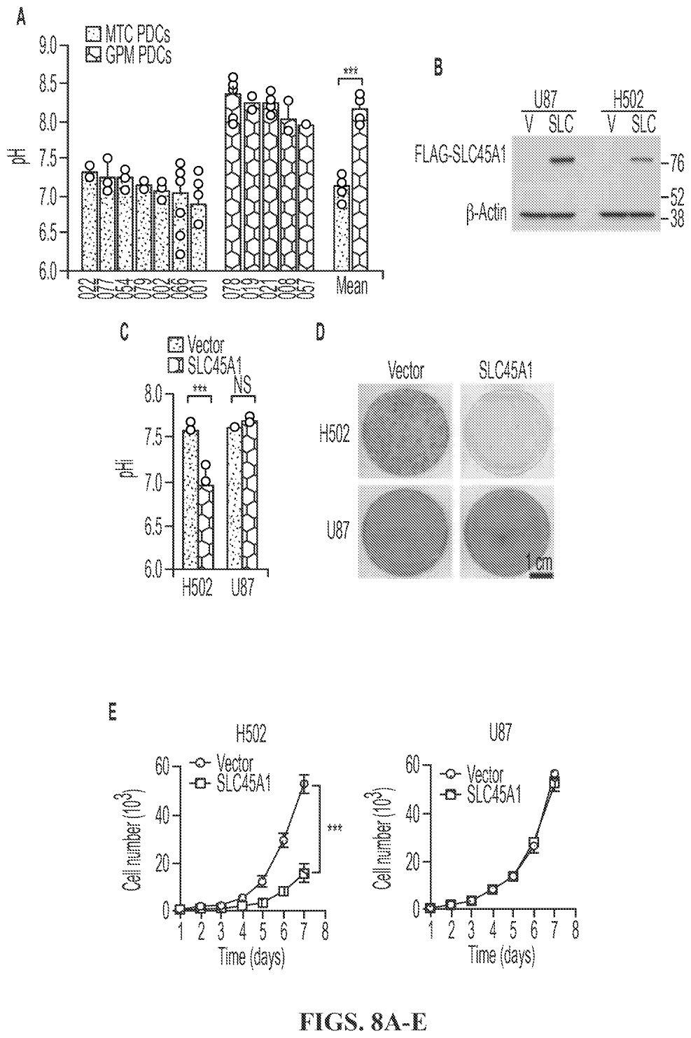

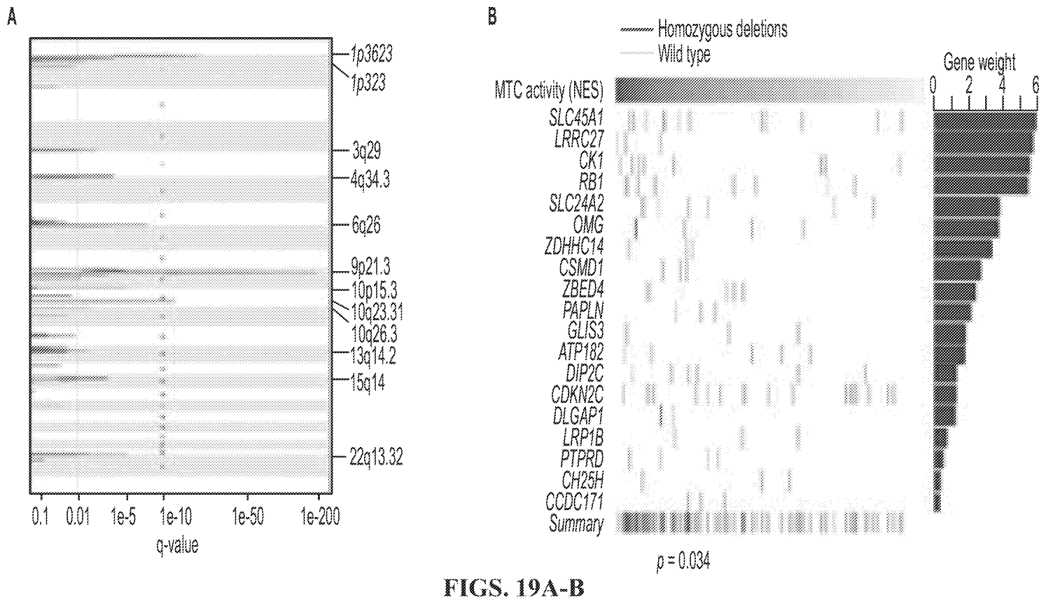

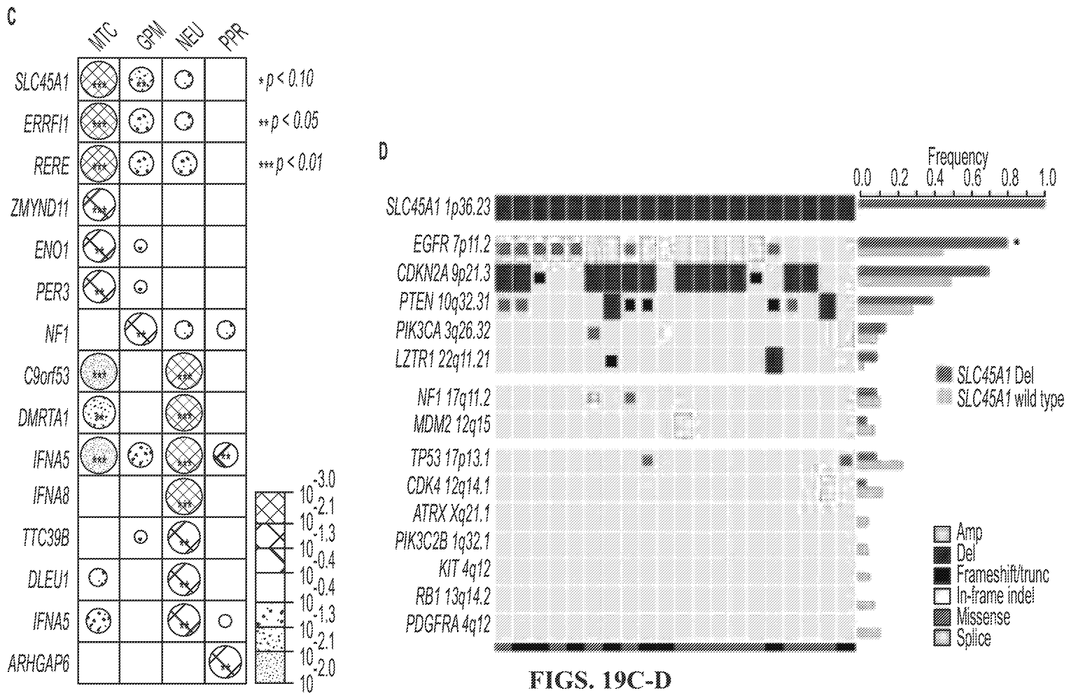

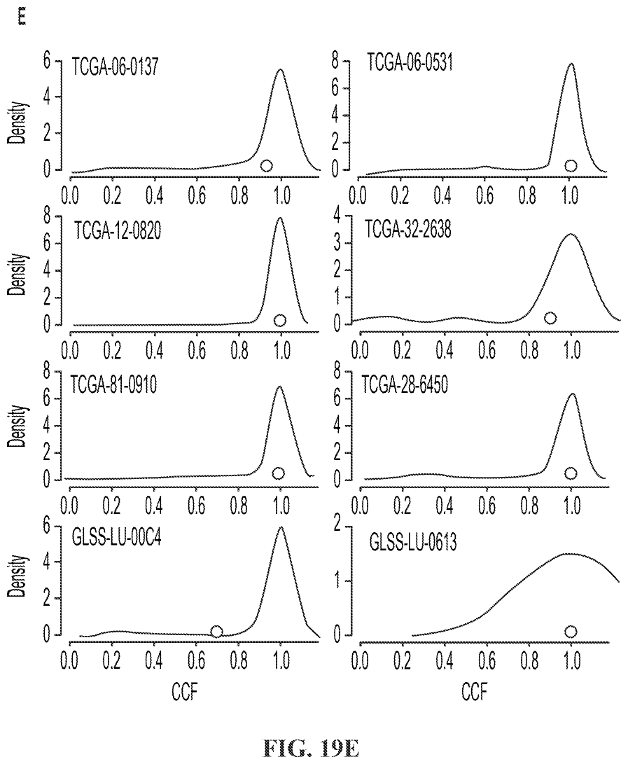

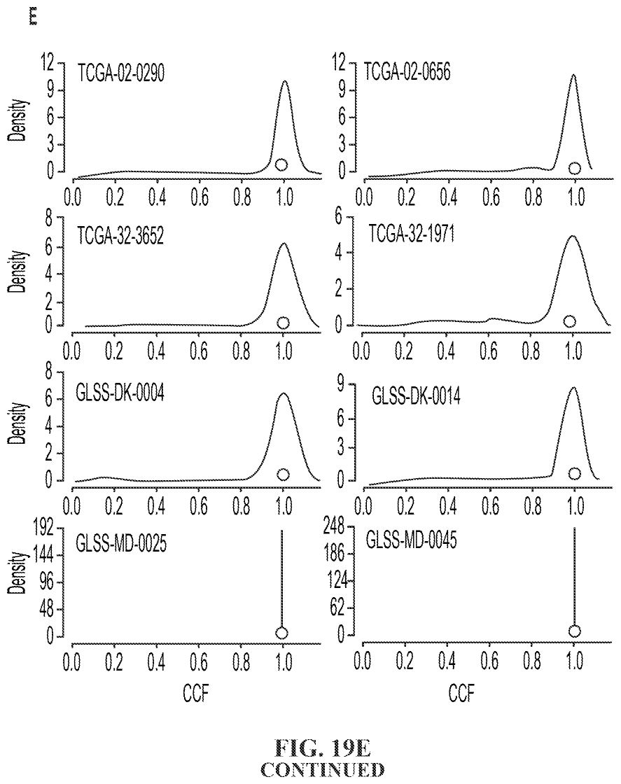

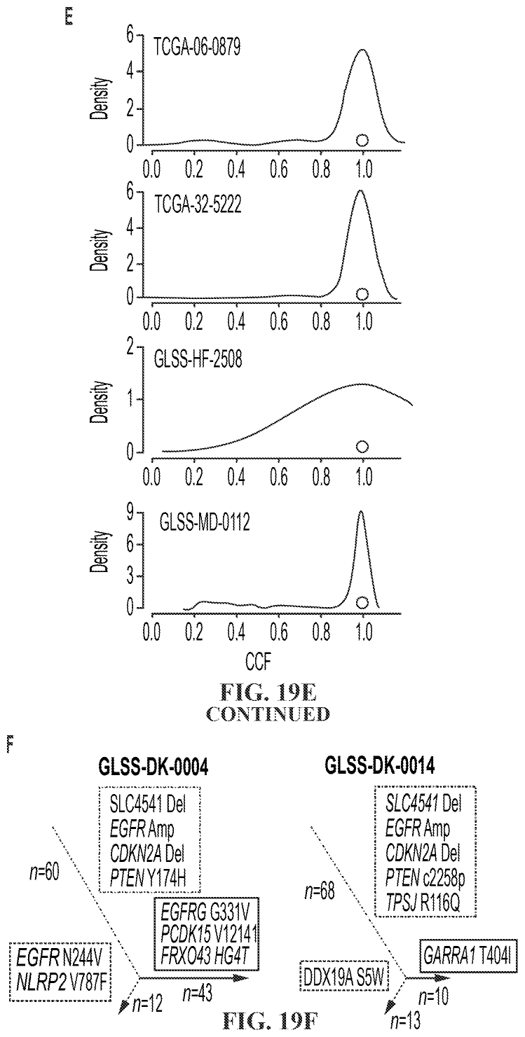

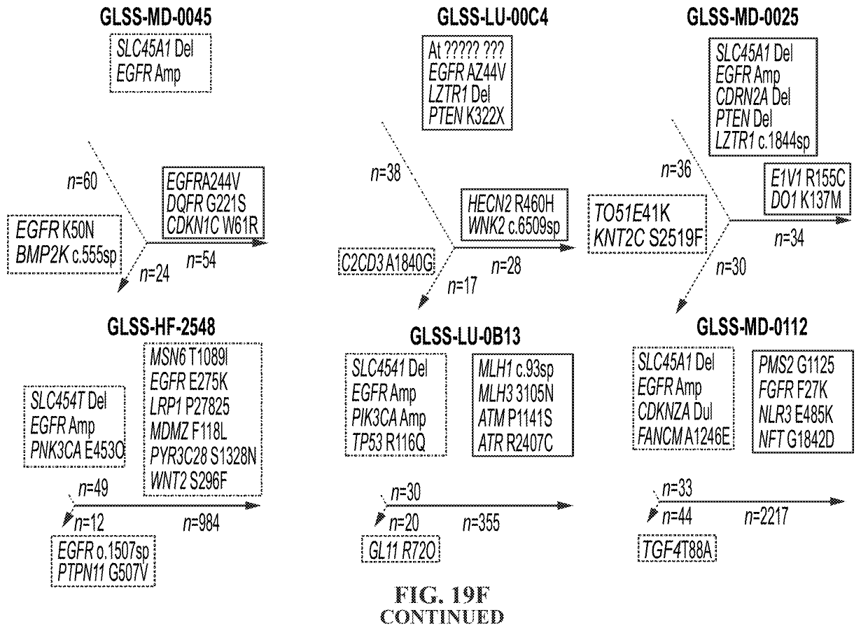

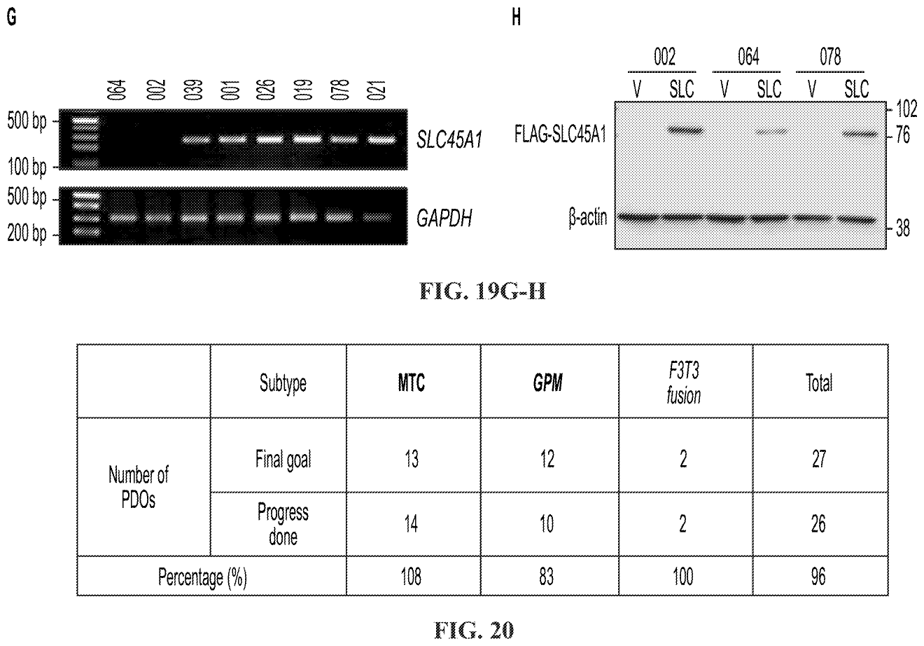

In addition to the wide spectrum of biologically coordinated genetic alterations driving GBM subtypes, GISTIC2 (ref 37) analysis performed to identify focal CNVs associated with each subtype revealed that MTC GBM harbored recurrent deletions of chromosome 1p36.23 (FIG. 19A). Chromosome 1p36.23 was also the top-ranking homozygous deletion, including genes with fCNV specifically associated with MTC compared with the other GBM subtypes (FIG. 7A). The chromosome 1p36.23 locus harbors several genes with known functions in glucose metabolism (ENO1, CA6, SLC2A5/GLUT5 and SLC2A7/GLUT7) among which the passenger deletion of ENO1 coding for the alpha-enolase glycolytic enzyme was found to generate therapeutic vulnerability in GB³⁸. To identify tumor suppressor genes driving 1p36.23 deletion in MTC GBM, we scored genes included in the 1p36.23-deleted region of MTC GBM with ComFocal, an algorithm that integrates recurrence with focality (FIG. 7B)³⁹ and applied this to the MTC profile of primary GBM UNCOVER, a computational tool for the identification of genetic alterations associated with cancer phenotypes (FIG. 19B)⁴⁰. We also used Fisher's exact test to globally score those genes harboring functional homozygous deletions associated with MTC GBM (FIG. 19C). The three approaches independently identified SLC45A1 as the top-ranking gene focally and functionally deleted in 1p36.23 in the MTC subtype. Consistently, the minimal 1p36.23 deletion in glioma cell linen H50241 encompassed SLC45A1 but did not affect ENO1 (FIG. 7C).

To determine the mutation landscape of SLC45A1-deleted tumors, we integrated genomic data of untreated IDH wild-type GBM from TCGA and GLASS42 and obtained a dataset of 725 tumors, 20 of which harbored homozygous deletion of SLC45A1. We compared the frequency of CNVs and SNVs in GBM driver genes in SLC45A1-deleted with that of SLC45A1 wild-type GBM and found that EGFR, CDKN2A, PTEN, PIK3CA and LZTR1 were more frequently altered in tumors with SLC45A1 deletions, TP53, CDK4, ATRX, PIK3C2B, KIT, RB1 and PDGFRA were targeted less frequently in SLC45A1-deleted GBM whereas NF1 and MDM2 alterations occurred at similar frequency in both groups (FIG. 19D). We also tracked the timing of SLC45A1 deletion in GBM evolution by determining the cancer cell fraction (CCF) in GBM harboring homozygous deletions of SLC45A1. SLC45A1 deletions were classified as clonal in each of the 20 tumors analyzed, thus indicating that loss of SLC45A1 is an early event in GBM evolution (FIG. 19E). Matched recurrent samples were available from eight of the 20 SLC45A1-deleted GBM, and deletions of SLC45A1 were retained at recurrence. To trace the evolutionary trajectory of SLC45A1-deleted GBM and determine the modules of genetic alteration associated with initiation and recurrence, we explored patient evolutionary trees and three-dimensional representation of alterations⁴³. We confirmed that SLC45A1 deletions are truncal and that the truncal module of SLC45A1-deleted GBM included EGFR amplification and alterations of CDKN2A, PTEN, PIK3CA and LZTR1. The genetic alterations that were progressively more specific for recurrent tumors included TP53, TEK, EGFR mutations, NF1, LRP1, ATR and PIK3C2B (FIG. 7D and FIG. 19F).

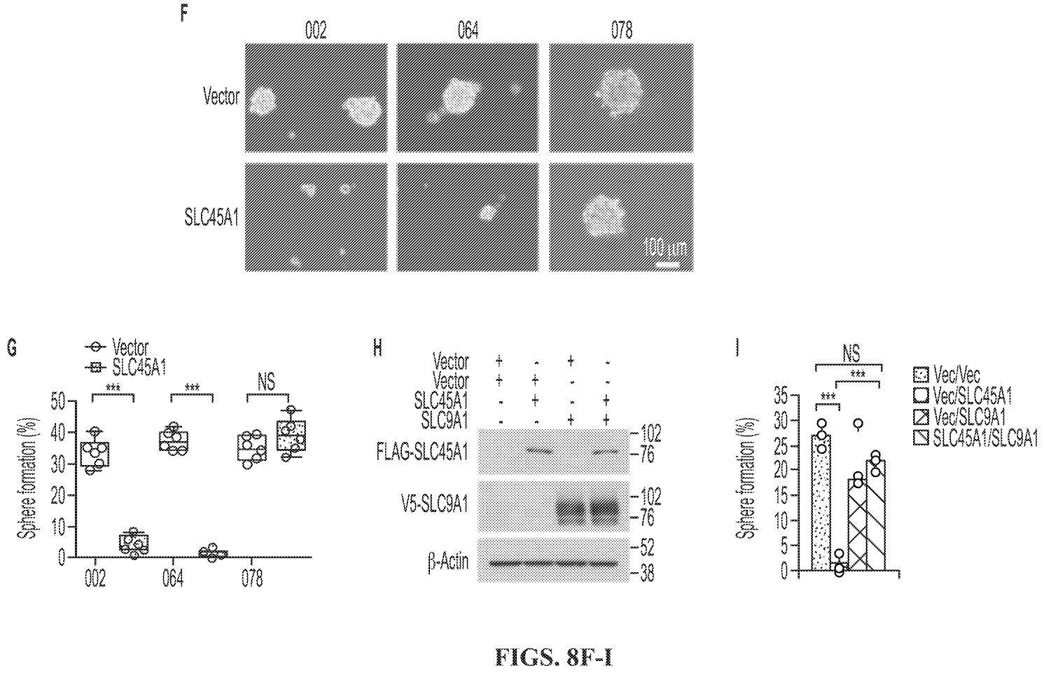

SLC45A1 encodes for a glucose-proton (H+) symporter that is specifically expressed in the central nervous system and transfers glucose and protons into the intracellular space. Loss-of-function mutations of SLC45A1 lead to a disorder characterized by neurodevelopmental disability due to impaired glucose transport⁴⁵. In cancer cells, the coupled intracellular proton-glucose transfer by SLC45A1 is predicted to counter the characteristic reversed pH gradient effected by multiple mechanisms of proton efflux that maintain an alkaline cytoplasmic pH⁴⁶. Most of the genes encoding for ion pumps and transporters that facilitate proton extrusion from cancer cells (for example, SLC9A1, SLC16A1/MCT1 and SLC16A3/MCT4 and CA9) are downregulated in MTC glioma cells and tumors whereas they are highly expressed in the GPM group (FIG. 7E). Consistent with this finding, intracellular pH (pHi) was lower in MTC than in GPM PDCs (MTC: pH 7.15, confidence interval (CI)=7.0-7.3; GPM: pH 8.17, CI=8.0-8.3; P=0.000004; FIG. 8A). We speculated that, with an already acidic intracellular environment, MTC GBM may not tolerate further decrease in pHi as result of the constant symporter activity of SLC45A1. Indeed, lentivirus-mediated re-expression of SLC45A1 in H502 cells (FIG. 8B) decreased pHi below 7.0 (FIG. 8C) and markedly impaired cell proliferation in colony-forming assays and growth kinetics (FIGS. 8D,E). Conversely, expression of SLC45A1 in U87 cells, which harbor an intact SLC45A1 locus (FIG. 7C), lacked discernible effects on either pHi or cell growth (FIGS. 8B-E). Next, we reintroduced SLC45A1 in MTC PDC-002 and -064, which harbor homozygous deletion of SLC45A1 (FIGS. 19G,H), and GPM PDC-078 lacking SLC45A1 alterations, and observed strong inhibition of gliomasphere formation in PDC-002 and -064 but no effect in PDC-078 (FIGS. 8F,G). Finally, we asked whether ectopic expression of the SLC9A1 proton extruder in PDC-002 would mitigate the negative effect on cell fitness caused by SLC45A1, and found that coexpression of SLC9A1 rescued self-renewal (FIGS. 8H,I).

### MTC GBM Exhibits Unique Vulnerability to OXPHOS Inhibition, Increased Sensitivity To Radiotherapy and Higher Intracellular ROS

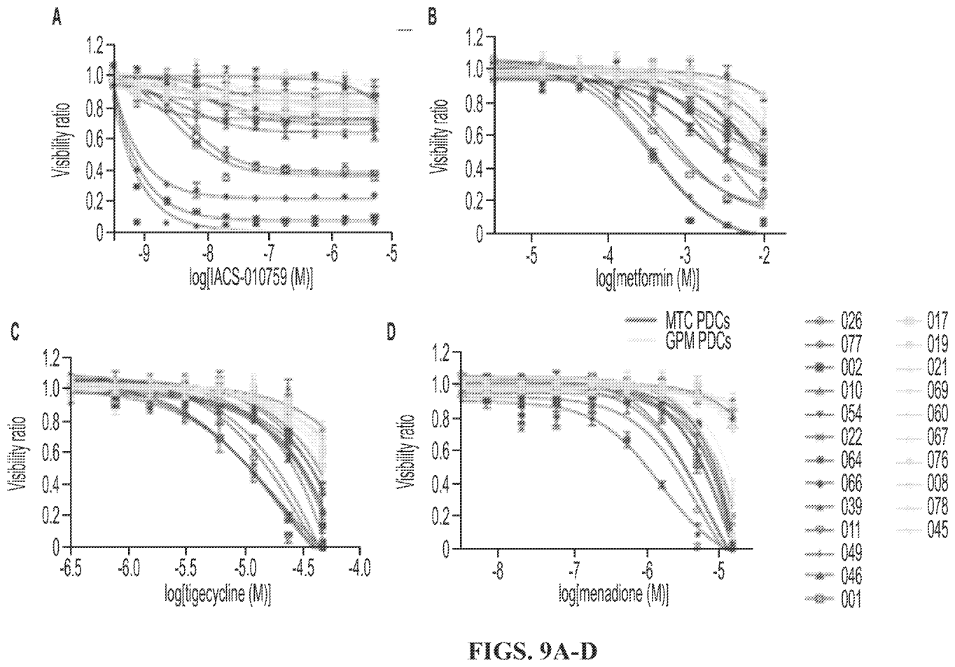

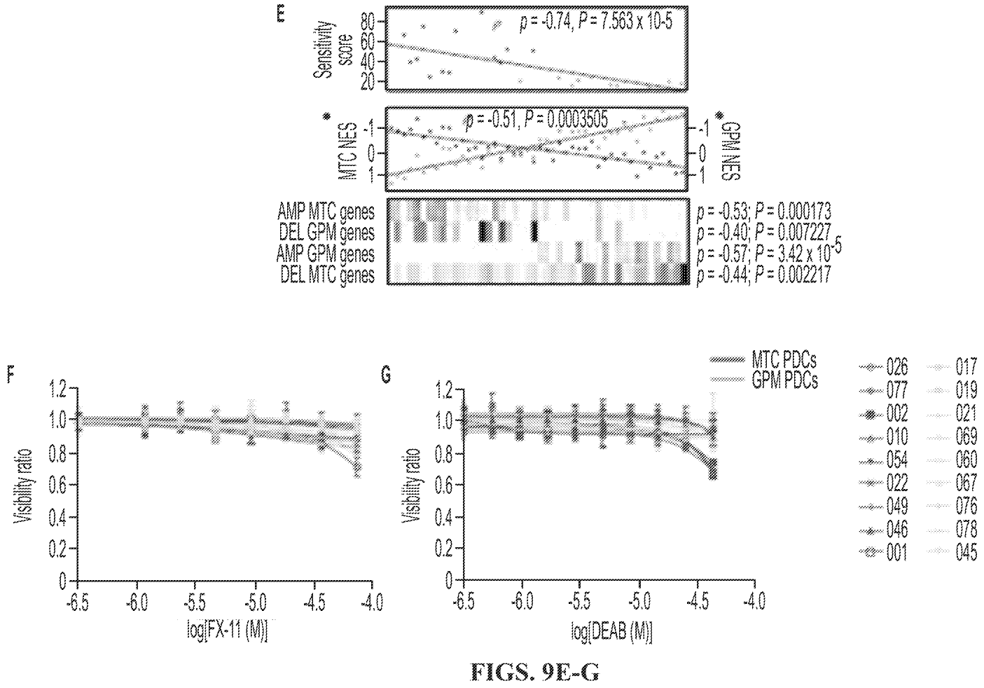

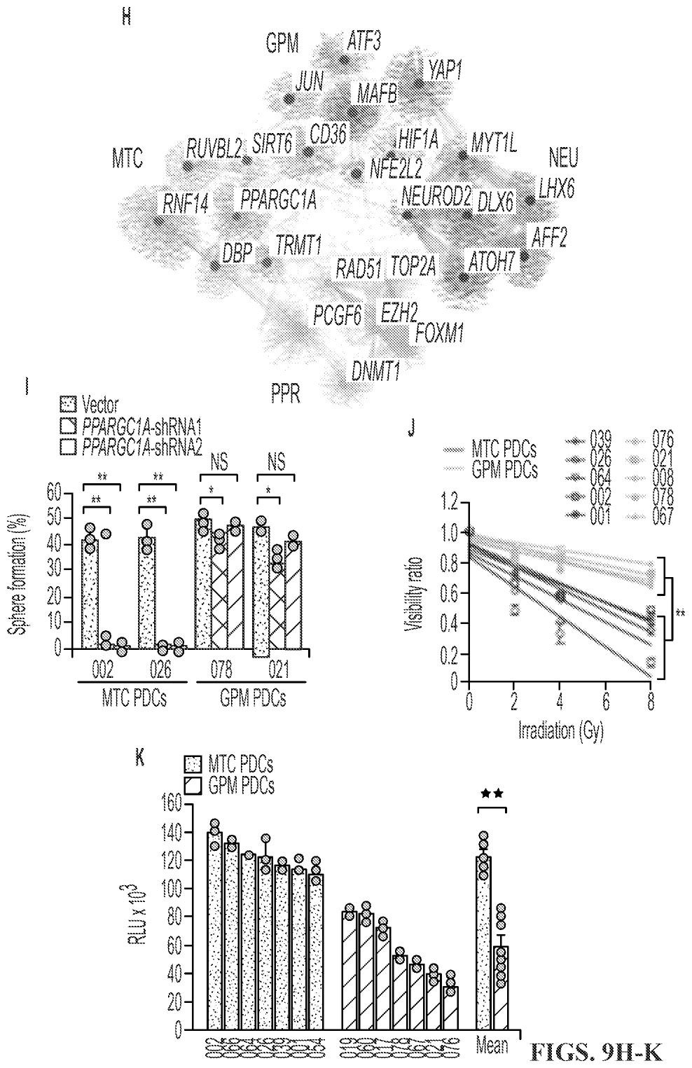

The finding that subtypes MTC and GPM GBM harbor reciprocal deletions and exhibit divergent metabolism suggested that the two metabolic GBM subgroups might harbor distinct therapeutic vulnerabilities. At variance with the metabolic redundancy of the GPM subtype, the exclusive reliance of MTC GBM upon OXPHOS for energy production suggested that a specific vulnerability might exist in this subtype. Therefore, we tested the sensitivity of 13 MTC and ten GPM PDCs to compounds that interfere with OXPHOS and mitochondrial metabolism. We used two inhibitors of mitochondrial complex I, metformin and IACS-010759, that decrease OXPHOS 47,48 ; tigecycline, inhibitor of mitochondrial protein translation⁴⁹; and menadione, inducer of mitochondrial ROS and apoptosis⁵⁰. p Mitochondrial inhibitors reduced the viability of MTC PDCs, albeit with variable potency and sensitivity in different PDCs (FIGS. 9A-D). Conversely, GPM PDCs were resistant to all four compounds (FIG. 9A-D). A sensitivity score that integrated the activity of the four mitochondrial inhibitors not only separated MTC PDCs (responders) from GPM PDCs (nonresponders; FIG. 9E) but also indicated that higher sensitivity positively correlated with MTC transcriptional activity and negatively with GPM activity (FIG. 9E). Furthermore, the complementary fCN gains and losses of MTC and GPM gene sets robustly correlated with mitochondrial inhibitor sensitivity score (FIG. 9E). Treatment of GBM PDCs with inhibitors of glycolysis (DEAB and FX-11)⁵¹ had no effect regardless of the metabolic class of PDC (FIGS. 9F,G). This finding indicates that activation of multiple metabolic pathways in GPM GBM probably creates a metabolic redundancy that generates tolerance to inhibition of glycolysis.

To provide independent validation of the sensitivity of MTC GBM organoids to inhibitors of mitochondrial metabolism, we interrogated the effects of silencing PGC1α (PPARGC1A), a MR of mitochondrial biogenesis and metabolism⁵². Interestingly, PGC1α scored as MTC GBM-specific MR from the differential analysis of MRs distinctly connected with the biological functions activated in each single-cell and bulk GBM subgroup (FIG. 9H). Silencing of PGC1α with two nonoverlapping shRNA lentiviruses¹⁴ was incompatible with self-renewal and growth of MTC GBM, but had only minimal effects in GPM PDCs (FIG. 9I), supporting the notion that mitochondrial metabolism is essential for the survival and growth of MTC GBM.

Because mitochondria, rather than the nucleus, are the primary organelle determining the effects of radiotherapy in cancer cells⁵³, we compared the sensitivity of MTC and GPM PDCs to radiotherapy, the standard of care for patients with GBM. MTC cells exhibited significantly higher sensitivity to radiotherapy treatment than GPM GBM (P=0.0022; FIG. 9J). These findings provide a clue to the better survival of patients with MTC GBM. Production of ROS by mitochondria is the primary source of oxidative stress induced by ionizing radiation⁵⁴. Accordingly, intracellular ROS were, on average, twofold higher in MTC than in GPM PDCs (P=0.00006; FIG. 9K), illuminating the probable mechanism responsible for the higher sensitivity of MTC GBM cells to oxidative stressors.

### Discussion

In this study, we present a transcriptional classification of IDH wild-type GBM based on the core biological functions denoting the identity of single glioma cells. A reproducible single-cell and bulk tumor typing was obtained when analyzing multiple datasets using a computational approach devised to measure pathway activities rather than gene signatures and supported by multiomics analyses. The pathway-based classifier segregated single glioma cells and primary GBM into four subtypes characterized by attributes of either development (NEU and PPR) or metabolism (MTC and GPM). With the inclusion of three separate scRNA-seq datasets, the computational approach was designed to overcome the challenges of batch integration by analysis of each tumor independently while combining downstream pathway measures. Nevertheless, the integrated analysis of scRNA-seq data obtained with different methods (droplet-based and full-length sequencing) remains a difficult task. Furthermore, as our approach focused primarily on glioma-cell-intrinsic biological states, future work will be required to explore in depth the heterogeneity within myeloid and other nontumor cell populations associated with each GBM subtype.

The pathway-based classification presented here introduces metabolism-associated GBM subtypes with prognostic and therapeutic implications for the MTC subgroup. It also adds an in-depth knowledge of the dynamics of neural cells within the neurodevelopmental axis of GBM. In this context, the PPR subgroup was enriched in tumor cells exhibiting neural progenitor features that coexist with the active cell cycle. Conversely, cells in the NEU subgroup expressed markers of neurons at various stages of maturation. Compared to previously established GBM classifiers^{3,11}, the discrimination of PPR and NEU groups paints a map of functions in GBM that recapitulate the transcriptional programs active at different stages of neurogenesis in the normal brain, from TBR1-positive newly born to differentiated neurons establishing synaptic connectivity^{9,55}. In contrast to a dynamic developmental core, the metabolic axis of GBM comprises two diverging metabolic states (MTC and GPM), sustained by opposing transcriptomic programs and genetic alterations generating a distinct metabolic dependency. Whereas the GPM subtype exhibited partial overlap with mesenchymal GBM, the MTC subtype defines a previously unknown glioma state that conveys prognostic and therapeutic information and is distributed orthogonally across the known subtypes. The hallmark features of two unique groups in the pathway-based classifier, subtypes PPR and MTC, can be generally distinguished by computational and metabolic analysis, respectively. For example, machine learning approaches were able to extract stem/progenitor cell indices from pan-cancer transcriptome⁵⁶. Conversely, in vivo metabolic studies have been used to identify functional mitochondrial heterogeneity within subtypes of lung cancer and it was proposed that these assays might also capture cancer metabolic vulnerabilities⁵⁷.

The classic concept that oncogenic and tumor suppression functions are mainly executed by genes targeted by focal CNVs has recently been challenged by observations documenting much broader cancer-promoting activities by CNVs associated with gene expression changes in cis⁵⁸⁻⁶⁰. By identifying congruent genetic alterations in each GBM subgroup, with fCNVs and SNVs targeting genes directly effecting a distinct subgroup phenotype, our work expands the definition of driver genes that in GBM have primarily been restricted to focal alterations. We also identified deletion of chromosome 1p36.23 as a genetic alteration distinctly associated with MTC GBM. The analysis of focality and experimental follow-up implicated the proton-glucose symporter SLC45A1 in a previously unknown mechanism of tumor suppression specific for the MTC state. We propose that activation of multiple proton extruders in GPM cells maintains an alkaline pHi that reinforces glycolysis and confers resistance to apoptotic signals. Conversely, a lower basal pHi renders MTC cells highly sensitive to further pH reduction. In this scenario, deletion of the proton-glucose symporter SLC45A1 is a necessary step to prevent unsustainable intracellular acidification.

The contrasting GPM and MTC subgroups of GBM are associated not only with gain-of-function alterations in genes promoting each particular metabolic state, but also with deletions and mutational inactivation of genes that implement the opposite phenotype. These findings offered unexpected opportunities for synthetic lethal therapeutics in MTC GBM. The obligate mitochondrial activity of MTC GBM boosted intracellular ROS, thus contributing to explaining the higher sensitivity of MTC PDCs to irradiation and the better clinical outcome in patients with MTC GBM. Conversely, the broad resistance of the GPM GBM subtype to multiple treatment types underpins the protective redundancy of metabolic activities in these tumors. Prominent among these, lipid biosynthesis and storage in lipid droplets represent a recognized protective mechanism in cancer cells³⁵.

The reciprocal MTC/GPM activity score captured the divergent biology of these GBM subtypes and predicted the therapeutic response of MTC PDCs to OXPHOS inhibition. The MTC/GPM activity score may be of general significance in multiple tumor types, and will be incorporated into new clinical studies testing the effect of OXPHOS inhibitors in patients with GBM.

### Methods

scRNA-seq datasets and sequencing. Single-cell gene expression profiles were collected from three datasets of primary human high-grade IDH wild-type glioma for a total of 36 tumors. The first dataset consists of nine grade IV gliomas (eight GBM and one gliosarcoma) and includes multisector biopsies obtained by precision navigator surgery⁶. The second dataset includes seven gliomas (six GBM and one grade III IDH wild-type glioma), four of which have previously been reported⁷, plus three specimens not previously reported (PJ053, PJ069 and PW032.706). The third dataset includes 20 adult IDH wild-type GBM specimens⁴. Single-cell RNA-seq libraries in dataset 1 were constructed following the single-cell tagged reverse transcription—seq protocol with minor modifications as previously described^{61,62}. Dataset 2 included GBM specimens dissociated and applied to an automated, microwell-based platform for scRNA-seq library construction⁷. Dataset 3 has been processed using Smart-Seq2 whole-transcriptome amplification, library construction and sequencing⁴. Raw sequencing reads of single cells were obtained from pooled library data by cell-specific barcodes. Sequences containing poly-A tails, sequencing adapters or low-quality bases (n bases >10%) were removed. Clean data were aligned to the GRCh38 human reference genome with STAR (v.2.0.5)⁶³. PCR redundant reads were eliminated by unique molecular identifier sequences, and the number of unique mapped reads on each gene was calculated with htseq-count⁶⁴.

scRNA-seq data processing and quality control. The final expression matrices include 4,227 cells (2,799 of which were malignant) for dataset 1, 10,315 (9,652 of which were malignant) for dataset 2 and 5,742 (4,916 of which were malignant) for dataset 3. A multistep approach to distinguish tumor from nontumor cells was applied.

Computational pipeline. Methods for the definition of single-sample pathway activity, scBiPaD, characterization of the biological states of single-cell subpopulations with single-cell gene metasignature, and subtype classification multiomics analyses of primary GBM are included, along with analyses of PDCs.

Impact of GBM functional subclasses on clinical outcome. Overall survival was calculated from the day of surgery to either the day of death or the end of follow-up. Kaplan-Meier survival curves were compared using the log-rank test. Cox's proportional hazards models were generated using the normalized enrichment score (NES) of each subtype as independent predictor. Cox's proportional hazards regression analysis was performed, including clinical and molecular covariates (age at diagnosis, gender, MGMT methylation status and functional GBM subtypes) individually or in combination. The association between functional groups and the clinical and molecular characteristics of GBM was performed using the χ2 test for binary covariates (gender, Karnofsky performance score, MGMT promoter status) and Kruskal-Wallis H-test with post hoc correction by Nemenyi's test for multiple subtype comparison of continuous variables (age and mutation count).

Validation datasets. We used tumor samples from three independent GBM cohorts and assigned each tumor to a distinct subtype on the basis of the highest significant score according to ssMWW-GST: log it(NES)>0.58 and FDR<0.01.

The first dataset consisted of 146 primary TCGA GBM IDH wild-type profiled by RNA-seq, of which 145 were available with survival data. Data were downloaded using the TCGA biolinks R/Bioconductor package⁶⁵. We applied genomic copy correction to the raw data for the within-normalization step and upper quantile for the between phase, according to a previously described pipeline⁶⁶. Out of 146 classified samples, 125 were also profiled with Agilent chip G4502A and this cohort was used in the cross-validation. A total of 86% of tumors received the same subtype across different platforms (for concordance, the union of unclassified samples in both platforms has been excluded from the total number considered).

The second dataset comprised 183 IDH wild-type GBM from the Chinese Glioma Genome Atlas (CGGA) cohort profiled by RNA-seq, of which 175 had survival data available67. Data were extracted from two batches of 325 and 693 gliomas of varying grade and histology, and corrected for batch effect using the COMBAT algorithm⁶⁸.

The third dataset included 219 GBM with available survival information (GEO: GSE13041) profiled with three different Affymetrix platforms (U133A, U133 Plus 2.0 and U95 v.2)⁶⁹. Probe intensities were converted to gene symbols, retaining only those genes covered by all platforms. Batch effects were corrected using the COMBAT algorithm while survival differences were assessed using the log-rank test.

Finally, we validated the 192 pathways associated with survival in the combined single-cell datasets by building the individual tumor consensus clustering with the NES of 19 survival-associated pathways. This analysis produced 103 subpopulations that clustered in K32 4 groups according to the Calinski—Harabasz criterion. Cluster assignment was confirmed by independent analysis of each single-cell dataset (˜94% concordance). Moreover, single-cell classification using a metasignature derived from the new subpopulation clusters revealed an overall concordance of subtype classification between the 5,032 and 192 pathway analyses of 91% for dataset 1, 93% for dataset 2 and 91% for dataset 3.

Analysis of the tumor microenvironment in GBM subclasses. Tumor purity of bulk GBM was evaluated using the ABSOLUTE inference method¹². Nontumor cells from single-cell cohorts were classified by ssMWW-GST using a collection of gene signatures of immune and stromal cells assembled from published reports^{3,7,13,70,71}.The association between nontumor cell types and functional GBM cell states was evaluated using Spearman's correlation. Enrichment of microglia or macrophages in the microenvironment of GPM and MTC tumors was tested in four GBM from dataset 1, two containing >75% GPM tumor cells (S4_D1 and S12_D1) and two comprising >75% MTC tumor cells (S1_D1 and S5_D1). For each individual myeloid cell, we defined a score calculating the difference in the expression mean of macrophage- and microglia-specific genes: S=μmacrophage−μtmicroglia. To highlight the macrophage- and microglia-specific genes whose differential expression mainly distinguished the two subpopulations, we correlated the expression of each gene with the score S across all cells. We selected and represented in the heatmap 25 genes with the highest (macrophage gene set) and the lowest (microglia gene set) correlation.

Assembly of transcriptional interactomes and MR analysis. To identify MRs of gene expression signatures activated in the four GBM subtypes, a context-specific transcriptional network from the Agilent gene expression profiles of 534 IDH wild-type GBM was assembled using the RGBM algorithm⁷². As input for the construction of the transcriptional network, a list of putative transcription regulators/factors (TFs) was derived from the Human Transcription Factors website including 2,765 proteins⁷³, a transcription factor list previously published⁷⁴, genes from the Ingenuity Pathways Knowledge Base and a list of known TFs from the TRANSFAC database^{75,76}. The list of putative TFs was further manually revisited, retaining those for which scientific evidence demonstrated their role as regulators of transcription. The list includes a total of 2,360 TFs expressed in the TCGA GBM IDH wild-type dataset. The transcriptional interactome comprised 210,468 (median regulon size, 147) interactions between 1,450 TFs (with at least 15 target genes) and 16,613 target genes. TF activity enrichment in each individual tumor or cell was computed by ssMWW-GST, as described in Pathway-based analysis of single glioma cells identifies four cellular states converging on two biological axes. We independently derived candidate MRs from the GBM TCGA cohort and the three single-cell datasets, and retained as significant (two-sided MWW-GST, FDR<0.01, log it(NES)>0.58; and two-sided MWW test for differential activity, FDR<0.01) only those MRs consistently activated in tumors and at least two single-cell datasets.

Plasmids, cloning and lentivirus production. Complementary DNAs for SLC45A1 and SLC9A1 were amplified by PCR and cloned into vectors pLVX and PLX, respectively, in-frame with the tag FLAG or V5. Lentivirus was produced by cotransfection of the lentiviral vectors with plasmids pCMV-ΔR8.1 and pCMV-MD2.G into HEK293T cells, as previously described¹⁴. Lentiviral vectors used for silencing of PPARGC1A were previously published¹⁴ and include the following sequences:

shPPARGCIA-Hs-1:
(SEQ ID NO: 1)
GCAGAGTATGACGATGGTATTCTCGAGAATACCATCGTCATACTCTGC
shPPARGC1A-Hs-2:
(SEQ ID NO: 2)
CCGTTATACCTGTGATGCTTTCTCGAGAAAGCATCACAGGTATAACGG.

Genomic DNA PCR. Genomic DNA from glioma cell lines and PDCs was assayed by semiquantitative PCR. Primer sequences are:

SLC45A1:
Fw
(SEQ ID NO: 3)
5′-AGGTCCCCATGGGATTGAGT-3′;
Rv
(SEQ ID NO: 4)
5′-GCACAATTGACAGCTGGGTC-3′
ENO1:
Fw
(SEQ ID NO: 5)
5′-TCACCTGTTGGCTACACAGAC-3′;
Rv
(SEQ ID NO: 6)
5′-CTTGGTGGAAAGTGAGGCGAG-3′.

Cell culture. The human cell lines used were U87 (ATCC HTB-14), HEK293T (ATCC CRL-11268), H502 and H423 (ref. 41). Cells were cultured as previously describe¹⁴.

Patient-derived cells were obtained using excess material collected for clinical purposes from deidentified brain tumor specimens. Donors (patients diagnosed with GBM) were anonymous. Work with these materials was designated as Institute for Research in Biomedicine (IRB) exempt under paragraph 4, and is covered under IRB protocol (no. IRB-AAAI7305) and Onconeurotek tumor bank certification (no. NF S96 900), and by authorization from the appropriate ethics committee (CPP Ile de France VI, ref. A3911) and the French Ministry for research (no. AC 2013-1962). PDCs were cultured and transduced, and gliomasphere assay was performed as described¹⁴.

Cell growth assay. Time course analysis of the cellular growth of H502 and U87 cells expressing SLC45A1 or the empty vector was performed by plating 1,000 cells per well in DMEM in six-well plates containing 10% fetal bovine serum. Viable cells were counted daily. Data are mean±s.d. of four replicates, and experiments were repeated twice. For clonogenic assay, 1,000 cells were plated in 60-mm² dishes. Cells were fixed in methanol and stained with crystal violet 2 weeks later. Photographs of one experiment performed in duplicate are presented. Experiments were repeated twice.

Quantification of pHi. Cells were plated at a density of 40,000-80,000 per 130 μl (four replicates) on opaque black 96-well plates. After 24-48 h, cells were stained using pHrodo Green AM Intracellular pH Indicator (Invitrogen, no. P35373) for 30 min at 37° C. Cells were washed with Fluoro Brite medium (Gibco, no. A18967-01) and immediately assayed using a multiplate fluorescence reader (VICTOR NIVO, Perkin Elmer) at 509-533-nm wavelength. pH calibration was obtained using the Intracellular pH Calibration Buffer Kit pH 4.5, pH 5.5, pH 6.5 and pH 7.5 (Invitrogen, no. P35379).

Metabolic assays. Measurement of OCR and extracellular acidification. The extracellular flux changes of oxygen and protons were measured using the XF96 Extracellular Flux Analyzer (Agilent) as previously described¹⁴.

Basal glycolysis indicates a normalized value of rate 4-8 (after glucose injection). Data are mean±s.d. from at least seven replicates in six MTC and six GPM PDCs, each derived from an independent patient. Exeriments were performed twice.

Intracellular glucose uptake, extracellular lactate concentration, glutamine consumption, triacylgliceride accumulation, lipid droplet visualization and ROS quantification. Measurement of the rate of glucose uptake, lactate secretion, glutamine consumption and triacylgliceride accumulation was performed using Glucose Uptake-Glo Assay (PROMEGA, no. J1342), Lactate-Glo Assay (PROMEGA, no. J5022), Glutamine/Glutamate-Glo Assay (PROMEGA, no. J5022) and Triglyceride-Glo Assay (PROMEGA, no. J3161), respectively, according to the manufacturer's instructions. Briefly, cells were plated at a density of 7,000 per 130 μl (three replicates) of medium containing 8 mM glucose and 2 mM glutamine in opaque white 96-well plates. Glucose uptake and lactate secretion were assayed 24 h after plating, while glutamine was measured at 36 h and triacylglicerides at 96 h. Luminescence was recorded at 0.3-s integration on a GloMax instrument. Data are mean±s.d. of triplicate observations from seven MTC and seven GPM PDCs from one representative experiment for glucose uptake, lactate secretion and triacylgliceride accumulation assays; and from two independent experiments for glutamine consumption.

Quantification of ROS was performed using ROS-Glo Assay (PROMEGA, no. G8821) according to the manufacturer's instructions. Briefly, cells were plated at a density of 7,000 per 80 μl (three replicates) and assayed 48 h later. Luminescence was recorded at 0.3-s integration on a GloMax instrument. Data are expressed as mean relative light units (RLU)±s.d. of triplicate observations from seven MTC and seven GPM PDCs from one representative experiment.

For visualization of lipid droplets in GBM PDCs, 30,000 cells were plated on laminin-coated glass coverslips; 96 h later, cells were washed with PBS and fixed in 3% paraformaldehyde for 15 min at room temperature. After two washes with PBS and quenching in 50 mM glycine, cells were stained with Bodipy 493/503 (Molecular Probes, no. D3922) at a concentration of 2 μg ml-1 for 30 min at room temperature. Coverslips were washed three times with PBS, counterstained with DAPI (Sigma) and mounted in Aqua Poly/Mount (Polysciences). Images were acquired under a ×60/0.9 numerical aperture objective Olympus 1×70 microscope equipped with a digital camera.

Compound treatment of PDCs. Cells were cultured in DMEM/F12 medium supplemented with N-2, B-27, EGF and FGF. Cells were plated in 130 μl in opaque white 96-well plates. Twenty-four hours later, cells were treated for 72 h with two- to threefold serial dilutions of selected compounds (FIG. 9) in six replicates. Viability was determined using CellTiterGlo assay reagent (Promega, no. G7570) and the GloMax-Multi+Microplate Multimode Reader (Promega).

Mitochondrial inhibitor sensitivity score. Patient-derived cells were treated with mitochondrial inhibitors (IACS-010759, metformin, tigecycline or menadione). The integrated score representative of the combined effect of the four drugs was obtained using the area under the curve (AUC) of dose—response for each individual drug. The mitochondrial sensitivity score (MSS) was defined as

$$MS \text{?} = \frac{1}{4} \text{?} \frac{1 - {AUC}_{ij}}{{max}_{j} ( 1 - {AUC}_{j} )}$$

$$\text{?} \text{indicates text missing or illegible when filed}$$

where i (i=1, . . . , n) represents the ith cell line, j (j=1, . . . , 4) represents the jth drug and the ratio

$$\frac{1 - {AUC}_{ij}}{{max}_{j} ( 1 - {AUC}_{j} )}$$

represents the normalized sensitivity score of the ith cell line to the jth drug such that the most responsive cell line for that drug was assigned a value of 100%.

Irradiation treatment of GBM PDCs. Patient-derived cells were plated in 96-well plates 24 h before radiation treatment. Cells were exposed to various irradiation doses (2, 4 and 8 Gy at 1.0 Gy min -1) from a 137Cs source (GammaCell 40 irradiator, Teratronics). Mock-irradiated cells were cultured in parallel. Viability was determined 96 h later using CellTiterGlo assay reagent (Promega, no. G7570) and the GloMax-Multi+Microplate Multimode Reader (Promega). Data are expressed as mean±s.d. of the viability ratio from six observations in five MTC and five GPM PDCs. Experiments were performed at least twice. Statistical significance was calculated from the value of slopes.

Immunoblot. Cells were lysed in RIPA buffer (50 mM Tris-HC1 pH 7.5, 150 mM NaCl, 1 mM EDTA, 1% NP40, 0.5% sodium dexoycholate, 0.1% sodium dodecyl sulfate, 1.5 mM Na₃VO₄, 50 mM sodium fluoride, 10 mM sodium pyrophosphate, 10 mM β-glycerolphosphate and EDTA-free protease inhibitor cocktail; Roche). Lysates were cleared by centrifugation at 15,000 r.p.m. for 15 min at 4° C., then separated by SDS-polyacrylamide gel electrophoresis and transferred to polyvinylidene difluoride membrane. Membranes were blocked in Tris-buffered saline with 5% nonfat milk and 0.1% Tween 20, and probed with primary antibodies overnight at 4° C. Antibodies and concentrations were as follows: FLAG 1:1,000 (Sigma, no. F1804), V5 1:1,000 (Invitrogen, no. R960-25) and β-actin 1:4,000 (Sigma, no. A5441). Secondary antibody anti-mouse conjugated horseradish peroxidase was purchased from Invtrogen (no. 31438), and either Enhanced ChemiLuminescence (Amersham, no. RPN2209) or Super Signal West Femto (Thermo Scientific, no. 34095) was used for detection.

Statistics and reproducibility. In general, at least two independent experiments were performed with a minimum of three biological replicates, as specified in figure legends. No statistical methods were used to predetermine sample size. No data were excluded from the analyses, the experiments were not randomized and the investigators were not blinded to allocation during experiments and outcome assessment. Comparisons between two groups were analyzed by either Welch's t (two-tailed, unequal variance) or two-sided MWW-GST test. Comparison between three or more groups was assessed by analysis of variance or Kruskal-Wallis test with Nemenyi post hoc correction for multiple comparison. Enrichment analysis of biological pathways was assessed using either two-sided MWW-GST or two-sided Fisher's exact test. Correlation analyses were performed using Spearman's correlation. Association between two groups was assessed by two-sided Fisher's exact test; association between three or more groups was assessed by the χ2 test. Survival differences were evaluated using the log-rank test or Cox's proportional hazards model. Results in graphs are expressed as either means±s.d. or means±s.e.m., as presented in figure legends for the indicated number of observations. Box plots span the first to third quartiles and whiskers show 1.5×interquartile range. All statistical analyses were performed and P values obtained using either GraphPad Prism software 6.0, R v.3.4.4, Jupyter Notebooks v.5.7.2 or Python v.3.6.

Additional methods are described in Garofano, L. et al. Pathway-based classification of glioblastoma uncovers a mitochondrial subtype with therapeutic vulnerabilities. Nat. Cancer. 2021, 2:141-156, which is incorporated herein in its entirety, Garofano, L. et al. Pathway-based classification of glioblastoma uncovers a mitochondrial subtype with therapeutic vulnerabilities. Supplementary Information. Nat. Cancer. 2021, 2:141-156, which is incorporated herein in its entirety, and Garofano, L. et al. Pathway-based classification of glioblastoma uncovers a mitochondrial subtype with therapeutic vulnerabilities. Supplementary Tables 1-18. Nat. Cancer. 2021, 2:141-156, which are incorporated herein in their entirety.

### References for Example 1

1. Cieslik, M. & Chinnaiyan, A. M. Cancer transcriptome profiling at the juncture of clinical translation. Nat. Rev. Genet. 19, 93-109 (2018).

2. Verhaak, R. G. et al. Integrated genomic analysis identifies clinically relevant subtypes of glioblastoma characterized by abnormalities in PDGFRA, IDH1, EGFR, and NF1. Cancer Cell 17, 98-110 (2010).

3. Wang, Q. et al. Tumor evolution of glioma-intrinsic gene expression subtypes associates with immunological changes in the microenvironment. Cancer Cell 32, 42-56 (2017).

4. Neftel, C. et al. An integrative model of cellular states, plasticity, and genetics for glioblastoma. Cell 178, 835-849 (2019).

5. Kim, S., Kon, M. & DeLisi, C. Pathway-based classification of cancer subtypes. Biol. Direct 7, 21 (2012).

6. Yu, K. et al. Surveying brain tumor heterogeneity by single-cell RNA-sequencing of multi-sector biopsies. Natl Sci. Rev. 7, 1306-1318 (2020).

7. Yuan, J. et al. Single-cell transcriptome analysis of lineage diversity in high-grade glioma. Genome Med. 10, 57 (2018).

8. Leone, G., Abla, H., Gasparre, G., Porcelli, A. M. & Iommarini, L. The Oncojanus paradigm of respiratory complex I. Genes (Basel) 9, 243 (2018).

9. Venkataramani, V. et al. Glutamatergic synaptic input to glioma cells drives brain tumour progression. Nature 573, 532-538 (2019).

10. Chen, H. et al. Single-cell trajectories reconstruction, exploration and mapping of omics data with STREAM. Nat. Commun. 10, 1903 (2019).

11. Phillips, H. S. et al. Molecular subclasses of high-grade glioma predict prognosis, delineate a pattern of disease progression, and resemble stages in neurogenesis. Cancer Cell 9, 157-173 (2006).

12. Carter, S. L. et al. Absolute quantification of somatic DNA alterations in human cancer. Nat. Biotechnol. 30, 413-421 (2012).

13. Caruso, F. P. et al. A map of tumor—host interactions in glioma at single-cell resolution. Gigascience 9, giaa109 (2020).

14. Frattini, V. et al. A metabolic function of FGFR3-TACC3 gene fusions in cancer. Nature 553, 222-227 (2018).

15. Zhang, J. et al. The combination of neoantigen quality and T lymphocyte infiltrates identifies glioblastomas with the longest survival. Commun. Biol. 2, 135 (2019).

16. Venteicher, A. S. et al. Decoupling genetics, lineages, and microenvironment in IDH-mutant gliomas by single-cell RNA-seq. Science 355, eaai8478 (2017).

17. Wang, J. et al. Clonal evolution of glioblastoma under therapy. Nat. Genet. 48, 768-776 (2016).

18. D'Angelo, F. et al. The molecular landscape of glioma in patients with neurofibromatosis 1. Nat. Med. 25,176-187 (2019).

19. Koh, E. H. et al. Mitochondrial activity in human white adipocytes is regulated by the ubiquitin carrier protein 9/microRNA-30a axis. J. Biol. Chem. 291,24747-24755 (2016).

20. Koh, E. H. et al. miR-30a remodels subcutaneous adipose tissue inflammation to improve insulin sensitivity in obesity. Diabetes 67,2541-2553 (2018).

21. Li, L. et al. miR-30a-5p suppresses breast tumor growth and metastasis through inhibition of LDHA-mediated Warburg effect. Cancer Lett. 400,89-98 (2017).

22. Chan, S. Y. et al. MicroRNA-210 controls mitochondrial metabolism during hypoxia by repressing the iron-sulfur cluster assembly proteins ISCU1/2. Cell Metab. 10,273-284 (2009).

23. Favaro, E. et al. MicroRNA-210 regulates mitochondrial free radical response to hypoxia and Krebs cycle in cancer cells by targeting iron sulfur cluster protein ISCU. PLoS ONE 5, e10345 (2010).

24. Papagiannakopoulos, T., Shapiro, A. & Kosik, K. S. MicroRNA-21 targets a network of key tumor-suppressive pathways in glioblastoma cells. Cancer Res. 68,8164-8172 (2008).

25. Bobbili, M. R., Mader, R. M., Grillari, J. & Dellago, H. OncomiR-17-5p: alarm signal in cancer? Oncotarget 8,71206-71222 (2017).

26. Sun, G. et al. miR-137 forms a regulatory loop with nuclear receptor TLX and LSD1 in neural stem cells. Nat. Commun. 2,529 (2011).

27. Liu, Y. et al. XBP1 silencing decreases glioma cell viability and glycolysis possibly by inhibiting HK2 expression. J. Neurooncol. 126,455-462 (2016).

28. Koo, J. H. & Guan, K. L. Interplay between YAP/TAZ and metabolism. Cell Metab. 28, 196-206 (2018).

29. Gao, Z. Y. et al. Metformin induces apoptosis via a mitochondria-mediated pathway in human breast cancer cells in vitro. Exp. Ther. Med. 11,1700-1706 (2016).

30. Hirata, T. et al. Stem cell factor induces outgrowth of c-kit-positive neurites and supports the survival of c-kit-positive neurons in dorsal root ganglia of mouse embryos. Development 119,49-56 (1993).

31. Strauss, B. et al. Cyclin B1 is essential for mitosis in mouse embryos, and its nuclear export sets the time for mitosis. J. Cell Biol. 217,179-193 (2018).

32. Gong, A. H. et al. FoxM1 drives a feed-forward STAT3-activation signaling loop that promotes the self-renewal and tumorigenicity of glioblastoma stem-like cells. Cancer Res. 75, 2337-2348 (2015).

33. Breiman, L. Random forests. Mach. Learn. 45, 5-32 (2001).

34. Cluntun, A. A., Lukey, M. J., Cerione, R. A. & Locasale, J. W. Glutamine metabolism in cancer: understanding the heterogeneity. Trends Cancer 3, 169-180 (2017).

35. Petan, T., Jarc, E. & Jusovic, M. Lipid droplets in cancer: guardians of fat in a stressful world. Molecules 23, 1941 (2018).

36. Fam, T. K., Klymchenko, A. S. & Collot, M. Recent advances in fluorescent probes for lipid droplets. Materials (Basel) 11, 1768 (2018).

37. Mermel, C. H. et al. GISTIC2.0 facilitates sensitive and confident localization of the targets of focal somatic copy-number alteration in human cancers. Genome Biol. 12, R41 (2011).

38. Muller, F. L. et al. Passenger deletions generate therapeutic vulnerabilities in cancer. Nature 488, 337-342 (2012).

39. Trifonov, V., Pasqualucci, L., Dalla Favera, R. & Rabadan, R. MutComFocal: an integrative approach to identifying recurrent and focal genomic alterations in tumor samples. BMC Syst. Biol. 7, 25 (2013).

40. Sarto Basso, R., Hochbaum, D. S. & Vandin, F. Efficient algorithms to discover alterations with complementary functional association in cancer. PLoS Comput. Biol. 15, e1006802 (2019).

41. Duncan, C. G. et al. Integrated genomic analyses identify ERRFIl and TACC3 as glioblastoma-targeted genes. Oncotarget 1, 265-277 (2010).

42. Barthel, F. P. et al. Longitudinal molecular trajectories of diffuse glioma in adults. Nature 576, 112-120 (2019).

43. Jolly, C. & Van Loo, P. Timing somatic events in the evolution of cancer. Genome Biol. 19, 95 (2018).

44. Shimokawa, N. et al. Past-A, a novel proton-associated sugar transporter, regulates glucose homeostasis in the brain. J. Neurosci. 22, 9160-9165 (2002).

45. Srour, M. et al. Dysfunction of the cerebral glucose transporter SLC45A1 in individuals with intellectual disability and epilepsy. Am. J. Hum. Genet. 100, 824-830 (2017).

46. Webb, B. A., Chimenti, M., Jacobson, M. P. & Barber, D. L. Dysregulated pH: a perfect storm for cancer progression. Nat. Rev. Cancer 11, 671-677 (2011).

47. Molina, J. R. et al. An inhibitor of oxidative phosphorylation exploits cancer vulnerability. Nat. Med. 24, 1036-1046 (2018).

48. Wheaton, W. W. et al. Metformin inhibits mitochondrial complex I of cancer cells to reduce tumorigenesis. eLife 3, e02242 (2014).

49. Skrtic, M. et al. Inhibition of mitochondrial translation as a therapeutic strategy for human acute myeloid leukemia. Cancer Cell 20, 674-688 (2011).

50. Criddle, D. N. et al. Menadione-induced reactive oxygen species generation via redox cycling promotes apoptosis of murine pancreatic acinar cells. J. Biol. Chem. 281, 40485-40492 (2006).

51. Altman, B. J., Stine, Z. E. & Dang, C. V. From Krebs to clinic: glutamine metabolism to cancer therapy. Nat. Rev. Cancer 16, 619-634 (2016).

52. Fernandez-Marcos, P. J. & Auwerx, J. Regulation of PGC-lalpha, a nodal regulator of mitochondrial biogenesis. Am. J. Clin. Nutr. 93, 884S-890S (2011).

53. Richardson, R. B. & Harper, M. E. Mitochondrial stress controls the radiosensitivity of the oxygen effect: implications for radiotherapy. Oncotarget 7, 21469-21483 (2016).

54. Kim, W. et al. Cellular stress responses in radiotherapy. Cells 8, 1105 (2019).

55. Venkatesh, H. S. et al. Electrical and synaptic integration of glioma into neural circuits. Nature 573, 539-545 (2019).

56. Malta, T. M. et al. Machine learning identifies stemness features associated with oncogenic dedifferentiation. Cell 173, 338-354 (2018).

57. Momcilovic, M. et al. In vivo imaging of mitochondrial membrane potential in non-small-cell lung cancer. Nature 575, 380-384 (2019).

58. Davoli, T. et al. Cumulative haploinsufficiency and triplosensitivity drive aneuploidy patterns and shape the cancer genome. Cell 155, 948-962 (2013).

59. Sack, L. M. et al. Profound tissue specificity in proliferation control underlies cancer drivers and aneuploidy patterns. Cell 173, 499-514 (2018).

60. Solimini, N. L. et al. Recurrent hemizygous deletions in cancers may optimize proliferative potential. Science 337, 104-109 (2012).

61. Dong, J. et al. Single-cell RNA-seq analysis unveils a prevalent epithelial/mesenchymal hybrid state during mouse organogenesis. Genome Biol. 19, 31 (2018).

62. Gao, S. et al. Tracing the temporal-spatial transcriptome landscapes of the human fetal digestive tract using single-cell RNA-sequencing. Nat. Cell Biol. 20, 721-734 (2018).

63. Dobin, A. & Gingeras, T. R. Mapping RNA-seq reads with STAR. Curr. Protoc Bioinformatics 51, 11.14.11-11.14.19 (2015).

64. Anders, S., Pyl, P. T. & Huber, W. HTSeq—a Python framework to work with high-throughput sequencing data. Bioinformatics 31, 166-169 (2015).

65. Colaprico, A. et al. TCGAbiolinks: an R/Bioconductor package for integrative analysis of TCGA data. Nucleic Acids Res. 44, e71 (2016).

66. Risso, D., Schwartz, K., Sherlock, G. & Dudoit, S. GC-content normalization for RNA-seq data. BMC Bioinformatics 12, 480 (2011).

67. Zhao, Z. et al. Comprehensive RNA-seq transcriptomic profiling in the malignant progression of gliomas. Sci Data 4, 170024 (2017).

68. Johnson, W. E., Li, C. & Rabinovic, A. Adjusting batch effects in microarray expression data using empirical Bayes methods. Biostatistics 8, 118-127 (2007).

69. Lee, Y. et al. Gene expression analysis of glioblastomas identifies the major molecular basis for the prognostic benefit of younger age. BMC Med. Genomics 1, 52 (2008).

70. Hussain, S. F. et al. The role of human glioma-infiltrating microglia/macrophages in mediating antitumor immune responses. Neuro. Oncol. 8, 261-279 (2006).

71. Quail, D. F. & Joyce, J. A. The microenvironmental landscape of brain tumors. Cancer Cell 31, 326-341 (2017).

72. Mall, R. et al. RGBM: regularized gradient boosting machines for identification of the transcriptional regulators of discrete glioma subtypes. Nucleic Acids Res. 46, e39 (2018).

73. Lambert, S. A. et al. The human transcription factors. Cell 175, 598-599 (2018).

74. Vaquerizas, J. M., Kummerfeld, S. K., Teichmann, S. A. & Luscombe, N. M. A census of human transcription factors: function, expression and evolution. Nat. Rev. Genet. 10, 252-263 (2009).

75. Wingender, E. The TRANSFAC project as an example of framework technology that supports the analysis of genomic regulation. Brief. Bioinformatics 9, 326-332 (2008).

76. Lee, S. B. et al. An ID2-dependent mechanism for VHL inactivation in cancer. Nature 529, 172-177 (2016).

### Example 2—Analysis of the Efficacy of the OXPHOS Inhibitor IM-156 in Glioblastoma Patients-Derived Organoids (PDOs)

The objective of the study is to test the activity of IM-156, a novel mitochondrial inhibitor currently in clinical testing, in vitro in 25 GBM-PDO organoids previously classified molecularly as mitochondrial (MTC, 13 organoids) or glycolytic/plurimetabolic (GPM, 12 organoids) plus 2 GBM-PDO harboring a FGFR3-TACC3 (F3T3) fusion. FIG. 20 shows distribution of glioblastoma patients-derived organoids (PDOs) by subtype for analysis of the efficacy of the OXPHOS inhibitor IM156.

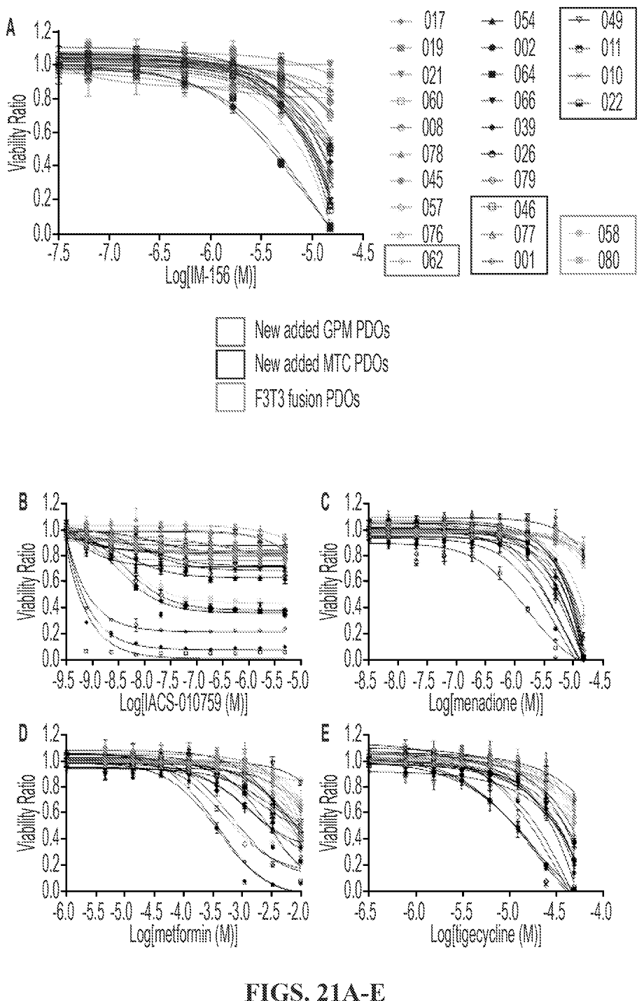

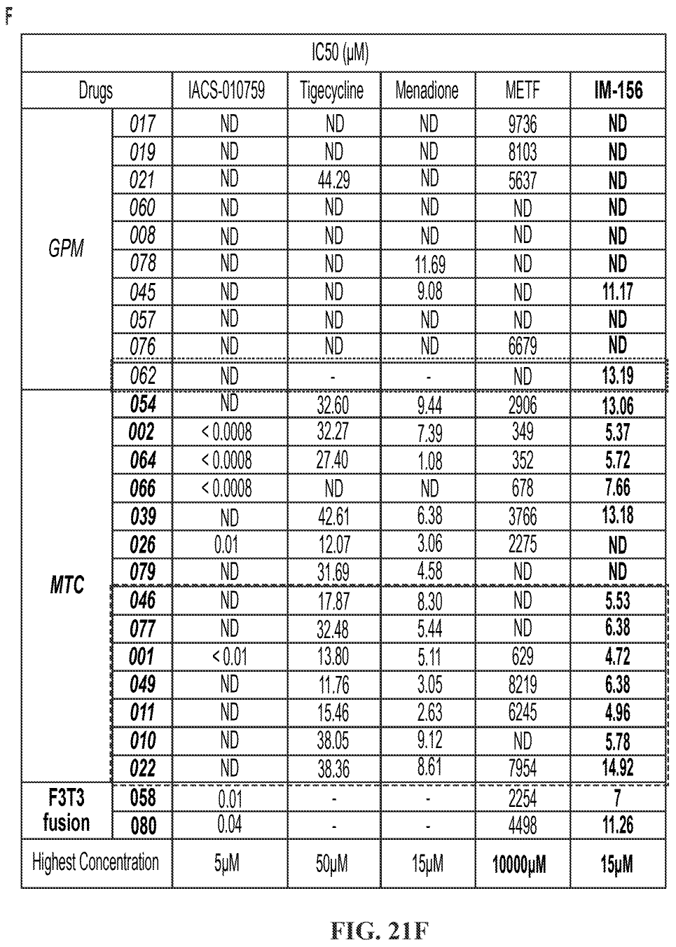

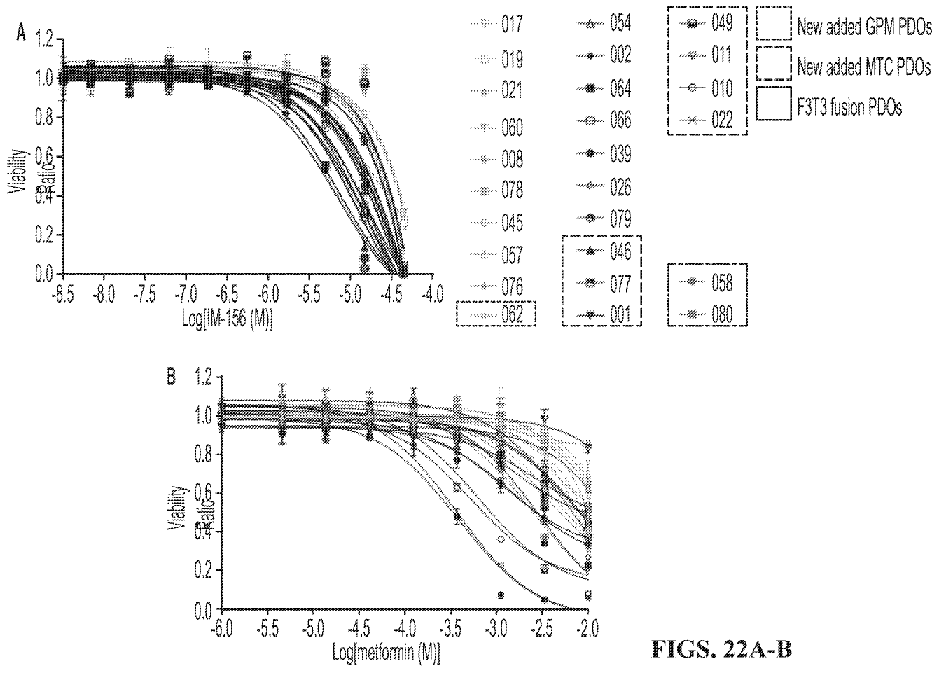

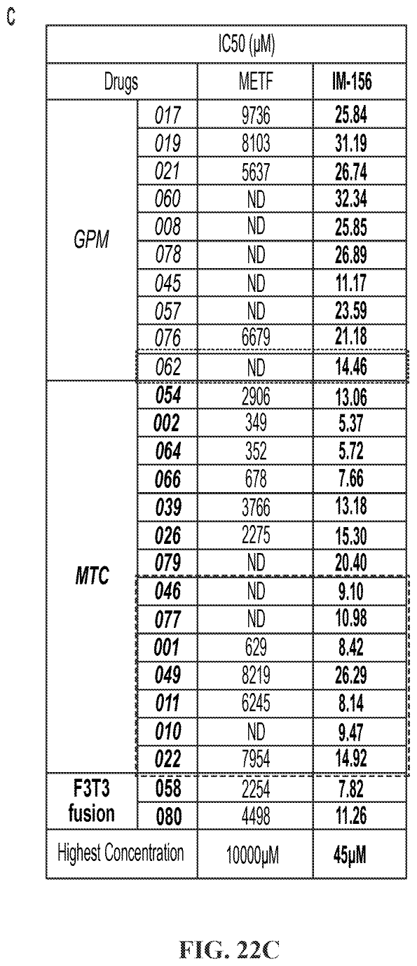

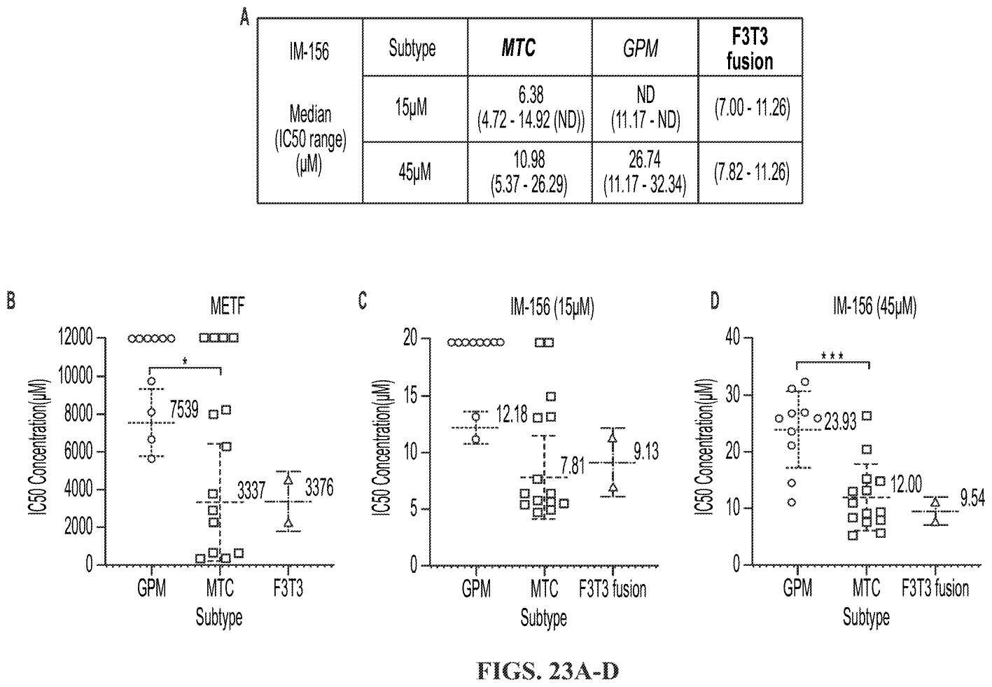

Glioblastoma patient-derived cells were exposed to serial dilution of mitochondrial inhibitors as indicated in FIGS. 21A-F and 22A-C. Using IM-156 (0 μM -15 μM, 1/3 dilution, 7 points and 0-45 μM, 1/3 dilution, 10 points), 14 mitochondrial, 10 glycolytic/plurimetabolic, and 2 FGFR3-TACC3 positive GBM PDOs were tested. Seventy-two hours later viability was assessed and the half-maximal inhibitory concentration was calculated. The activity (IC₅₀) of IM-156 was compared with IACS-010759, Metformin, Tigecycline, and Menadione, 4 inhibitors of mitochondrial activity/respiration. When used at 15 μM as the highest concentration, IM-156 was effective in 12 out of 14 (Median IC50: 6.38 μM) mitochondrial PDOs, 2 out of 10 glycolytic/plurimetabolic PDOs, and 2 out of 2 FGFR3-TACC3 PDOs, as shown in FIGS. 23A-D. Increasing maximum concentration of the drug caused a significant increase in sensitivity of the glycolytic/plurimetabolic GBM PDOs. IM-156 is significantly more effective at targeting glioblastoma patients-derived tumor models classified as mitochondrial (MTC), as opposed to other glioblastoma subtypes (glycolytic/plurimetabolic or GPM). This indicates that mitochondrial classified Glioblastoma patient derived cells are more sensitive to mitochondrial inhibition than glycolytic/plurimetabolic cells. FGFR3-TACC3 fusion expressing cells had been previously characterized as mitochondrial GBM. Accordingly, they both exhibited distinct sensitivity to IM-156. These data indicate that IM-156 can be used to treat patients with mitochondrial glioblastoma.

What is claimed is:

1. A method of treating glioblastoma (GBM) in a subject in need thereof, the method comprising:
providing a GBM sample from the subject;
determining a GBM subtype for the GBM sample; and
administering to the subject a pharmaceutical composition wherein the pharmaceutical composition modifies activity of one or more functional pathway associated with the GBM subtype.

2. The method of claim 1, wherein the GBM is IDH wild-type GBM.

3. The method of claim 1, wherein the GBM subtype is a neurodevelopmental subtype.

4. The method of claim 3, wherein the GBM subtype is neuronal (NEU).

5. The method of claim 3, wherein the GBM subtype is proliferative/progenitor (PPR).

6. The method of claim 1, wherein the GBM subtype is a metabolic subtype.

7. The method of claim 6, wherein the GBM subtype is mitochondrial (MTC).

8. The method of claim 7, wherein the MTC GBM subtype harbors deletions in at least a portion of chromosome 1p36.23.

9. The method of claim 8, wherein the deletions in at least a portion of chromosome 1p36.23 comprise a deletion of gene SLC45A1.

10. The method of claim 6, wherein the GBM subtype is glycolytic/plurimetabolic (GPM).

11. The method of claim 1, wherein the GBM subtype comprises an FGFR3-TACC3 gene fusion.

12. The method of claim 1, wherein the pharmaceutical composition is an inhibitor of mitochondrial metabolism.

13. The method of claim 1, wherein the pharmaceutical composition is an inhibitor of mitochondrial activity.

14. The method of claim 1, wherein the pharmaceutical composition is an inhibitor of mitochondrial respiration.

15. The method of claim 1, wherein the pharmaceutical composition is an OXPHOS inhibitor.

16. The method of claim 1, wherein the pharmaceutical composition is IM-156.

17. The method of claim 1, wherein the pharmaceutical composition is an inhibitor of mitochondrial complex I.

18. The method of claim 1, wherein the pharmaceutical composition is metformin.

19. The method of claim 1, wherein the pharmaceutical composition is IACS-010759.

20. The method of claim 1, wherein the pharmaceutical composition is tigecycline.

21. The method of claim 1, wherein the pharmaceutical composition is an inhibitor of mitochondrial protein translation.

22. The method of claim 1, wherein the pharmaceutical composition is menadione.

23. The method of claim 1 wherein, the pharmaceutical composition is an inducer of mitochondrial ROS or apoptosis.

24. The method of claim 1, wherein the determining comprises a single cell RNA-seq analysis of the sample.

25. The method of claims 1, wherein the determining comprises a scBiPaD analysis of the sample.

26. The method of claim 1, wherein the determining comprises defining cluster-specific ranked-lists.

27. The method of claims 1, wherein the determining comprises consensus clustering analysis of cell subpopulations in the sample.

28. The method of claim 1, wherein the determining comprises:
generating a gene signature of the sample;
comparing the gene signature to one or more gene signatures of GBM samples with known subtype;
making a determination of the GBM subtype based on matching the gene signature to one or more known gene signature.

29. The method of any one of claims 24-29, wherein the determining further comprises a genomic analysis, a transcriptomic analysis, a DNA methylation analysis, a microRNA analysis, or a proteomics analysis of the sample.

30. A method of a determining clinical outcome in a subject having glioblastoma (GBM), the method comprising:
providing a GBM sample from the subject;
determining a GBM subtype for the GBM sample; and
providing a clinical outcome based on the GBM subtype.

31. The method of claim 30, wherein the GBM is IDH wild-type GBM.

32. The method of claim 30, wherein the GBM subtype is a neurodevelopmental subtype.

33. The method of claim 32, wherein the GBM subtype is neuronal (NEU).

34. The method of claim 32, wherein the GBM subtype is proliferative/progenitor (PPR).

35. The method of claim 30, wherein the GBM subtype is a metabolic subtype.

36. The method of claim 35, wherein the GBM subtype is mitochondrial (MTC).

37. The method of claim 36, wherein the MTC GBM subtype harbors deletions in at least a portion of chromosome 1p36.23.

38. The method of claim 37, wherein the deletions in at least a portion of chromosome 1p36.23 comprise a deletion of gene SLC45A1.

39. The method of claim 35, wherein the GBM subtype is glycolytic/plurimetabolic (GPM).

40. The method of claim 30, wherein the determining comprises a single cell RNA-seq analysis of the sample.

41. The method of claims 30, wherein the determining comprises a scBiPaD analysis of the sample.

42. The method of claim 30, wherein the determining comprises defining cluster-specific ranked-lists.

43. The method of claims 30, wherein the determining comprises consensus clustering analysis of cell subpopulations in the sample.

44. The method of claim 30, wherein the determining comprises:
generating a gene signature of the sample;
comparing the gene signature to one or more gene signatures of GBM samples with known subtype;
making a determination of the GBM subtype based on matching the gene signature to one or more known gene signature.

45. The method of any one of claims 40-44, wherein the determining further comprises a genomic analysis, a transcriptomic analysis, a DNA methylation analysis, a microRNA analysis, or a proteomics analysis of the sample.


SEQUENCE LISTING (6 sequences)

SEQ ID NO: 1 - DNA, 48 bp, Artificial Sequence
  Description of Artificial Sequence Synthetic oligonucleotide
gcagagtatgacgatggtattctcgagaataccatcgtcatactctgc

SEQ ID NO: 2 - DNA, 48 bp, Artificial Sequence
  Description of Artificial Sequence Synthetic oligonucleotide
ccgttatacctgtgatgctttctcgagaaagcatcacaggtataacgg

SEQ ID NO: 3 - DNA, 20 bp, Artificial Sequence
  Description of Artificial Sequence Synthetic primer
aggtccccatgggattgagt

SEQ ID NO: 4 - DNA, 20 bp, Artificial Sequence
  Description of Artificial Sequence Synthetic primer
gcacaattgacagctgggtc

SEQ ID NO: 5 - DNA, 21 bp, Artificial Sequence
  Description of Artificial Sequence Synthetic primer
tcacctgttggctacacagac

SEQ ID NO: 6 - DNA, 21 bp, Artificial Sequence
  Description of Artificial Sequence Synthetic primer
cttggtggaaagtgaggcgag